(19) United States
(12) Patent Application Publication
(10) Pub. No.: US 2023/0022614 A1
(43) Pub. Date: Jan. 26, 2023

(54) POLYMERIC FATTY ACID SALT COMPOUNDS FOR THE TREATMENT OF FIBROUS AMINO ACID-BASED SUBSTRATES, ESPECIALLY HAIR

(71) Applicant: Momentive Performance Materials GmbH, Leverkusen (DE)

(72) Inventors: Roland Wagner, Bonn (DE); Katharina Streicher, Leverkusen (DE); Laura Rohmann, Aachen (DE); Christian Wenske, Solingen (DE); Shrutisagar Dattatraya Haveli, Maharashtra (IN)

(21) Appl. No.: 17/840,922

(22) Filed: Jun. 15, 2022

Related U.S. Application Data
(60) Provisional application No. 63/211,095, filed on Jun. 16, 2021.

(51) Int. Cl. A61K 8/41; A61Q 5/00

(52) U.S. Cl. CPC A61K 8/416; A61Q 5/00

(57) ABSTRACT

The present invention is directed at organic ammonium salts comprising

an organic ammonium-group-comprising cation which does not contain an estolide moiety and a carboxylate anion (COO—) group-comprising anion selected from the group consisting of anions containing at least one estolide moiety or carboxylate anions containing at least one internal ester group.

The invention is also directed at a process for the production of such organic ammonium salts, the use of such organic ammonium salts in cosmetic formulations for skin and hair care, the use of such organic ammonium salts for the treatment of fibers, the use of the organic ammonium salts for the treatment of hair, at compositions containing such organic ammonium salts, the use of the products of the process for the production of organic ammonium salts in cosmetic formulations for skin care and hair conditioner and hair shampoo, the use of said products for the treatment of fibers, and compositions containing said products for the treatment of hair.

This application claims priority to U.S. Provisional Application No. 63/211,095 file on Jun. 16, 2021, the entire contents of which are incorporated herein by reference.

### FIELD OF THE INVENTION

This invention relates to polymeric fatty acid salt compounds, a process for their production, compositions containing the salt compounds, and the use of the salt compounds in cosmetic compositions comprising the same for skin and hair care, in particular, hair care compositions, and their use for the treatment of hair.

### BACKGROUND OF THE INVENTION

Hair generally can be straight, wavy, curly, kinky or twisted. A human hair includes three main morphological components, the cuticle (a thin, outer-most shell of several concentric layers), the cortex (the main body of the hair), and, in case of higher diameter hair, the medulla (a thin, central core). The cuticle and cortex provide the hair strand's mechanical properties, that is, its tendency to have a wave, curl, or kink. A straight hair strand can resemble a rod with a circular cross-section, a wavy hair strand can appear compressed into an oval cross-section, a curly strand can appear further compressed into an elongated ellipse cross-section, and a kinky hair strand cross-section can be flatter still.

The primary component of hair is the cross-linked, alpha-helix protein keratin. Keratins are intermediate filament proteins found specifically in epithelial cells, e.g. human skin and hair, wool, feathers, and nails. The α-helical type I and II keratin intermediate filament proteins (KIFs) with molecular weights around 45-60 kDa are embedded in an amorphous matrix of keratin-associated proteins (KAPs) with molecular weights between 20 to 30 kDa (M. A. Rogers, L. Langbein, S. Praetzel-Wunder, H. Winter, J. Schweizer, J. Int Rev Cytol. 2006; 251:209-6); both intra- and intermolecular disulfide bonds provided by cystines contribute to the cytoskeletal protein network maintaining the cellular scaffolding. In addition to the disulfide cross-links ionic bonding or salt bridges which pair various amino acids found in the hair proteins contribute to the hair strand's outward shape.

It is known in the art that hair can be treated with functionalized silicones and hydrocarbons which deliver one or more cosmetic benefits, such as conditioning, shine and UV protection as well as color retention. Typically, these silicones and hydrocarbon-based derivatives are physically deposited on the fiber surface (cuticle) and therefore responsible for the outward appearance of the hair, i.e. smoothness, silkiness, friction, alignment and combability.

Advanced silicone derivatives are generally regarded as high performing materials with respect to attributes such as smooth and silky hair feel, friction reduction, eased combability and hair color protection. Respective quaternized silicones are described in prior art disclosures, i.e. in U.S. Pat. No. 4,891,166, EP 282720, US 2008027202, U.S. Pat. Nos. 6,730,766, 6,240,929, WO 02/10257, WO 02/10259, WO 2004/069137, WO 2013/148629, WO 2013/148635, WO 2013/148935.

Hydrocarbon-based conditioning agents are also widely used. Typically, mono quaternary ammonium compounds are mono-long alkyl—tri short alkyl quaternized ammonium salts or di-long alkyl-di short alkyl quaternized ammonium salts wherein one or two alkyl substituents are selected from an aliphatic group of from about 8 to about 30 carbon atoms or an aromatic, alkoxy, polyoxyalkylene, alkylamido, hydroxyalkyl, aryl or alkylaryl group having up to about 30 carbon atoms; the other alkyl groups are independently selected from an aliphatic group of from about 1 to about 8 carbon atoms or an aromatic, alkoxy, polyoxyalkylene, alkylamido, hydroxyalkyl, aryl or alkylaryl group having up to about 8 carbon atoms; and the counter ion is a salt-forming anion such as those selected from halogen, (e.g., chloride, bromide), acetate, citrate, lactate, glycolate, phosphate, nitrate, sulfonate, sulfate, alkylsulfate, glutamate, and alkyl sulfonate radicals. Alternatively, these mono quaternary ammonium compounds are saturated or unsaturated fatty acid-based mono-fatty ester and di-fatty ester quats as well as fatty amido quats having 10 to 24 carbon atoms in the alkyl chain(s). Details on these materials containing quaternary ammonium groups are disclosed for example in US 2009/0000638, WO 2012/027369, US 2013/259820 and U.S. Pat. Nos. 5,880,086, 6,465,419, 6,462,014, 6,323,167, 6,037,315, 5,854,201, 5,750,490, 5,463,094, US 2003/013627.

Di-quaternized hydrocarbons are also known. Typically, these gemini quats are based on C8 to C20 alkyl or fatty chains (D. Shukla et. al., Cationic Gemini Surfactants: A Review, Journal of Oleo Science 2006, Vol. 55, Nr. 8, 381-390; M. J. Rosen et. al. Langmuir (2001), 17, 6148-6154).

Di-quaternized hydrocarbons based on an alternating copolyester of castor oil and different dicarboxylic acids are described in US 2003/0007950 and U.S. Pat. No. 6,972,123.

A castor oil precursor was used to synthesize a material containing three quat groups (EP 0283994, A. Baydar et. al., International Journal of Cosmetic Science (1991), 13(4), 169-90).

Dimers of fatty acids were used to synthesize polyquaternary fatty acid dimer copolymers (U.S. Pat. No. 6,982,078).

WO 2004/093834 describes hydrocarbon based mono quaternary compounds for personal care applications. These compounds mandatorily contain linkers having the structure —CH₂CH₂O-EOₓ—PO_{y}—. Polymerized fatty acids were proposed as hydrophobic tails.

U.S. Pat. No. 6,051,214 proposes shampoos containing as key ingredients cleansing surfactants, thickeners, water and estolides. Further, conditioners are proposed containing conditioning agents, thickeners, water and estolides.

There has been a need for efficient compounds for the treatment of fibrous amino acid based substrates, especially hair which can be synthesized in a straight forward, cost efficient and flexible way, largely based on sustainable raw materials, which are easy to formulate and easy to use, yielding long term stable formulations even in the presence of other performance ingredients and which are useful for the conditioning of hair, for an improved dry and wet combability of hair, the smoothness and a pleasant alignment of hair. In particular, benefits regarding an improved wet and dry combability close to silicone based conditioning agents should be achieved.

The present inventors found that new polymeric fatty acid-based salt compounds comprising carboxylate anions of estolide structures, and aqueous compositions comprising such salt compounds are suitable to satisfy the above need. The present invention accordingly provides new polymeric fatty acid based salt compounds comprising carboxylate anions containing estolide structures, aqueous compositions comprising the same, cosmetic compositions comprising the same, in particular, hair care compositions, and their use for the treatment of hair, which polymeric fatty acid based salt compounds comprising carboxylate anions containing estolide structures can be synthesized in a straightforward, cost-efficient and flexible way, largely based on sustainable raw materials. The compounds of the invention are easy to formulate and to use, and are useful for the conditioning of hair, for an improved dry and wet combability of hair, the smoothness and a pleasant alignment of hair.

### SUMMARY OF THE INVENTION

This invention relates to fatty acid based organic ammonium salts in which the carboxylate anions are based on estolide structures, while the cations do not display such structures, as well as a process for their production, compositions containing the salt compounds, and the use of the salt compounds in cosmetic compositions comprising the same for skin and hair care, in particular, hair care compositions, and their use for the treatment of hair.

Accordingly, with the present invention it is provided an organic ammonium salt comprising an organic ammonium-group-comprising cation,

with the proviso that said organic ammonium-group-comprising cation does not comprise moieties of the formulas (III) or (IV):

(—Z—C(O)—R⁶)ᵣ—Z—C(O)— (III) or

(—C(O)—Z—R⁶)ᵣ—C(O)—Z— (IV),

wherein
Z can be the same or different and is selected from ¿O—, or —NR¹¹—, wherein
R¹¹ is independently selected from the group consisting of hydrogen, or optionally substituted straight-chain, cyclic or branched, saturated, unsaturated or aromatic hydrocarbon radicals which have up to 100 carbon atoms which optionally contain one or more groups selected from
—O—, —NH—, —C(O)—, —C(S)—, tertiary amino groups

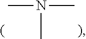

and can be substituted with one or more hydroxyl and halide groups,
R⁶ is independently selected from optionally substituted straight-chain, cyclic or branched, saturated or unsaturated hydrocarbon radicals which have 1 to 36 carbon atoms, with the proviso that at least one R⁶ has more than 6 carbon atoms, and
r is 1 to 20,
and a carboxylate anion (COO⁻) group-comprising anion selected from the group consisting of anions of the formulae (V), (VII), and (X):

formula (V):

R⁷(—X—C(O)-G)ₚ (V)

wherein
R⁷ in formula (V) is selected from a p-valent, optionally substituted hydrocarbon radical and may contain optionally one or more groups selected from —O—, —NH—, —C(O)—, —C(S)—, tertiary amino groups

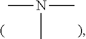

and quaternary ammonium groups, and can be optionally substituted by one or more substituent groups selected from a carboxyl group (—COOH) group, a carboxylate anion (—COO⁻) group and a hydroxyl (—OH) group,
p≥1, more preferably 2-811,
X can be the same or different and is selected from —O—, or —NR¹⁰—, wherein R¹⁰ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

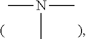

or in formula (V) R¹⁹ may form a bond to R⁷ to form a cyclic structure,
G can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups

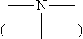

and can be optionally substituted by one or more substituent groups selected from a carboxyl (—COOH) group, a carboxylate anion (—COO⁻) group, a hydroxyl (—OH) group and a halide (-halogen) group, with the proviso that at least one of the radicals G contains at least one moiety of the formula (VI) or (VI*):

—R⁸(—X—C(O)—R⁸)ₘ—X—C(O)—R⁹ (VI)

—R⁸(—X—C(O)—R⁸)ₘ—X—C(O)—R^{9*} (VI*)

wherein
X is as defined above,
m=0 to 20, preferably 1 to 20,
R⁸ is independently selected from a divalent optionally substituted straight-chain, cyclic or branched, saturated or unsaturated hydrocarbon radical which have up to 36 carbon atoms,
R⁹ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups

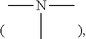

quaternary ammonium groups

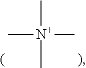

and which can be substituted with one or more substituent groups selected from a carboxyl (—COOH) group, carboxylate anion (—COO⁻) group, a hydroxyl (—OH) group, and a halide (-halogen) group, wherein the radical R⁹ cannot contain an internal carboxy (—COO—) group or (—CON(R′)—, R′ being hydrogen or organic group) amide group, i.e. R⁹ cannot contain a combination of a —C(O)— group and a —O— group or a combination of a —C(O)— group and a —NH— or tertiary amino group, and
with the proviso that in at least one moiety of the formula (VI) R⁹ has at least 2, preferably at least 6 carbon atoms, and that in the same moiety of the formula (VI) at least one R⁸ has at least 6, preferably at least 8 carbon atoms,
with the proviso, that at least one of R⁷ and G comprises one or more carboxylate anion (—COO⁻) groups,
R⁹* is independently selected from optionally substituted branched or dendrimeric hydrocarbon radicals which have 1 to 1000 carbon atoms, optionally containing one or more groups selected from —O—, —NH—, —C(O)—, —C(S)—, tertiary amino groups

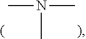

quaternary ammonium groups

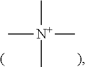

and which can be substituted with carboxyl, hydroxyl, or halide groups, wherein the radical R^{9*} is terminated by two or more groups of the general structure

—X—C(O)-T

wherein X is as defined above, and
T is a monovalent straight-chain, cyclic or branched, saturated or unsaturated hydrocarbon radical optionally substituted with carboxyl, hydroxyl, or halide groups with up to 36 carbon atoms,
with the proviso that in at least one moiety of the formula (VI*) R⁹* is terminated by one or more groups T having at least 2, preferably at least 6 carbon atoms, and in the same moiety of the formula (VI*) at least one R⁸ has at least 6, preferably at least 8 carbon atoms,
and with the proviso, that at least one of R⁷ and G in formula (V) comprises one or more carboxylate anion
(—COO⁻) groups,

formula (VII):

R⁷(—C(O)—X—Y)_{q} (VII),

wherein
R⁷ and X are as defined above,
q=1 to 55, preferably 1 to 40, more preferably 2 to 4, and
Y can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—,
—C(O)—, —C(S)—, and tertiary amino groups

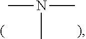

and can be substituted by one or more substituent groups selected from a carboxyl (—COOH) group, a carboxylate anion (—COO⁻) group and a hydroxyl group,
with the proviso that at least one of the radicals Y contains at least one moiety of the formula (VIII) or (VIII*):

—R⁸(—C(O)—X—R⁸)ₘ—C(O)—X—R⁹ (VIII)

—R⁸(—C(O)—X—R⁸)ₘ—C(O)—X—R^{9*} (VIII*)

wherein X, m, R⁸, and R⁹ in formula (VIII) are each as defined above for formula (VI), and X, m, R⁸, and R^{9′} in formula (VIII*) are each as defined above for formula (VI*), and with the proviso that at least one of R⁷ and Y in formula (VII) comprises one or more carboxylate anion (—COO—) groups,

R⁷(—C(O)—X—R⁸—COO⁻)_{q} formula (X):

wherein X, R⁷, R⁸, and q in formula (X) are each as defined above for formula (VII) and (VIII).

### DETAILED DESCRIPTION OF THE INVENTION

In the following, the invention is described in detail.

According to the present invention, it is provided an organic ammonium salt comprising

an organic ammonium-group-comprising cation,
with the proviso that said organic ammonium-group-comprising cation does not comprise moieties of the formulas (III) or (IV):

(—Z—C(O)—R⁶)ᵣ—Z—C(O)— (III) or

(—C(O)—Z—R⁶)ᵣ—C(O)—Z— (IV),

wherein
Z can be the same or different and is selected from —O—, or —NR¹¹—, wherein
R¹¹ is independently selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

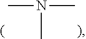

and can be substituted with one or more hydroxyl and halide groups,
R⁶ is independently selected from optionally substituted straight-chain, cyclic or branched, saturated or unsaturated hydrocarbon radicals which have 1 to 36 carbon atoms, with the proviso that at least one R⁶ has more than 6 carbon atoms, and
r is 1 to 20,
and a carboxylate anion (COO⁻) group-comprising anion selected from the group consisting of anions of the formulae (V), (VII), and (X):

formula (V):

R⁷(—X—C(O)-G)ₚ (V)

wherein
R⁷ in formula (V) is selected from a p-valent, optionally substituted hydrocarbon radical and may contain optionally one or more groups selected from —O—, —NH—, —C(O)—, —C(S)—, tertiary amino groups

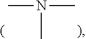

and quaternary ammonium groups, and can be optionally substituted by one or more substituent groups selected from a carboxyl group (—COOH) group, a carboxylate anion (—COO⁻) group and a hydroxyl (—OH) group,
p≥1, more preferably 2-811,
X can be the same or different and is selected from —O—, or —NR¹⁰—, wherein R¹⁰ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

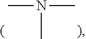

or in formula (V) R¹⁰ may form a bond to R⁷ to form a cyclic structure,
G can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups

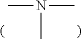

and can be optionally substituted by one or more substituent groups selected from a carboxyl (—COOH) group, a carboxylate anion (—COO⁻) group, a hydroxyl (—OH) group and a halide (-halogen) group, with the proviso that at least one of the radicals G contains at least one moiety of the formula (VI) or (VI*):

—R⁸(—X—C(O)—R⁸)ₘ—X—C(O)—R⁹ (VI)

—R⁸(—X—C(O)—R⁸)ₘ—X—C(O)—R^{9*} (VI*)

wherein
X is as defined above,
m=0 to 20, preferably 1 to 20,
R⁸ is independently selected from a divalent optionally substituted straight-chain, cyclic or branched, saturated or unsaturated hydrocarbon radical which have up to 36 carbon atoms,
R⁹ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups

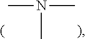

quaternary ammonium groups

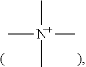

and which can be substituted with one or more substituent groups selected from a carboxyl (—COOH) group, carboxylate anion (—COO⁻) group, a hydroxyl (—OH) group, and a halide (-halogen) group, wherein the radical R⁹ cannot contain an internal carboxy (—COO—) group or (—CON(R′)—, R′ being hydrogen or organic group) amide group, i.e. R⁹ cannot contain a combination of a —C(O)— group and a —O— group or a combination of a —C(O)— group and a —NH— or tertiary amino group, and
with the proviso that in at least one moiety of the formula (VI) R⁹ has at least 2, preferably at least 6 carbon atoms, and that in the same moiety of the formula (VI) at least one R⁸ has at least 6, preferably at least 8 carbon atoms,
with the proviso, that at least one of R⁷ and G comprises one or more carboxylate anion (—COO⁻) groups,
R⁹* is independently selected from optionally substituted branched or dendrimeric hydrocarbon radicals which have 1 to 1000 carbon atoms, optionally containing one or more groups selected from —O—, —NH—, —C(O)—, —C(S)—, tertiary amino groups

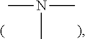

quaternary ammonium groups

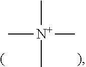

and which can be substituted with carboxyl, hydroxyl, or halide groups, wherein the radical R^{9*} is terminated by two or more groups of the general structure

—X—C(O)-T

wherein X is as defined above, and
T is a monovalent straight-chain, cyclic or branched, saturated or unsaturated hydrocarbon radical optionally substituted with carboxyl, hydroxyl, or halide groups with up to 36 carbon atoms,
with the proviso that in at least one moiety of the formula (VI*) R⁹* is terminated by one or more groups T having at least 2, preferably at least 6 carbon atoms, and in the same moiety of the formula (VI*) at least one R⁸ has at least 6, preferably at least 8 carbon atoms,
and with the proviso, that at least one of R⁷ and G in formula (V) comprises one or more carboxylate anion
(—COO⁻) groups,

formula (VII):

R⁷(—C(O)—X—Y)_{q} (VII),

wherein
R⁷ and X are as defined above,
q=1 to 55, preferably 1 to 40, more preferably 2 to 4, and
Y can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups

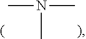

and can be substituted by one or more substituent groups selected from a carboxyl (—COOH) group, a carboxylate anion (—COO⁻) group and a hydroxyl group,
with the proviso that at least one of the radicals Y contains at least one moiety of the formula (VIII) or (VIII*):

—R⁸(—C(O)—X—R⁸)ₘ—C(O)—X—R⁹ (VIII)

—R⁸(—C(O)—X—R⁸)ₘ—C(O)—X—R^{9*} (VIII*)

wherein X, m, R⁸, and R⁹ in formula (VIII) are each as defined above for formula (VI), and X, m, R⁸, and R^{9*} in formula (VIII*) are each as defined above for formula (VI*),
and with the proviso that at least one of R⁷ and Y in formula (VII) comprises one or more carboxylate anion (—COO—) groups,

R⁷(—C(O)—X—R⁸—COO⁻)_{q} formula (X):

wherein X, R⁷, R⁸, and q in formula (X) are each as defined above for formula (VII) and (VIII).

According to the invention, estolides are natural and synthetic compounds, in particular derived from fats and oils, more specifically from the fatty acid compounds typically obtainable by hydrolysis of oils and fats,

The estolide structure is identified by the secondary ester linkage of one fatty acyl molecule to the alkyl backbone of another fatty acid fragment. The terms “fatty acid” and “fatty acyl molecule” seem to imply that the individual residue needs to be derived from a component of a fat, which is not the case. The term “fatty acid” herein refers to carboxylic acids with chain-shaped organyl groups, in particular unbranched aliphatic monocarboxylic acids. Fatty acids differ from each other by their number of carbon atoms (chain length) and, when referring to unsaturated fatty acids, the number and position of double bonds. Fatty acids may be classified as short chain fatty acids with up to 7 carbons atoms, middle chain fatty acids with 8 to 12 carbon atoms, long chain fatty acids with 13 to 21 carbon atoms, and very long chain fatty acids with more than 22 carbon atoms.

According to the invention, in general the group “—O—” represents an ether group, which also includes the presence of an epoxide moiety, which is a tri-membered cyclic ether group. Accordingly, the groups defined above as optionally comprising the group “—O—” may contain epoxy groups.

In general, the features of all embodiments according to the invention as described below can be combined freely unless otherwise noted or the combination of features or incorporation of isolated features of an embodiment into another embodiment is not possible by definition of the parameters, i.e. because they are incompatible by the virtue of logic.

According to the invention, an organic ammonium-group is any group comprising a quaternary nitrogen atom which is bonded directly to at least one C atom of an organyl group, i.e. of any organic substituent group, regardless of functional type, having one free valence at a carbon atom.

According to the invention, the organic ammonium salt cation or cations, if the salt comprises more than one organic ammonium cation, do not comprise moieties of the formulas (III) or (IV):

(—Z—C(O)—R⁶)ᵣ—Z—C(O)— (III) or

(—C(O)—Z—R⁶)ᵣ—C(O)—Z— (IV).

The group R⁶ can be the same or different selected from optionally substituted straight-chain, cyclic or branched, saturated or unsaturated hydrocarbon radicals which have 1 to 36 carbon atoms, and can thus represent a hydrocarbyl group selected from the group consisting of linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 22 carbon atoms,

There is no limitation regarding at which C-atoms of the hydrocarbyl radicals the adjacent group C(O) group and Z group are bonded to R⁶.

Z can be the same or different and is selected from O or NR¹¹, wherein R¹¹ is independently selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

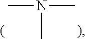

and can be substituted with one or more hydroxyl and halide groups.

The number r of the R⁶-containing repeating units (—Z—C(O)—R⁶) or (—C(O)—Z—R⁶) of the moieties defined by formula (III) or formula (IV) excluded from the cationic structure is from 1 to 20.

The organic ammonium salt according to the invention comprises at least one carboxylate anion (COO⁻) group-comprising anion selected from the group consisting of anions of the formulas (V), (VII), and (X) as defined above.

The structure of the anions of the formula (V)

R⁷(—X—C(O)-G)ₚ (V)

is defined as follows:

According to the invention, in formula (V) the residue R⁷ is p-valent, wherein p is ≥1 to 811, preferably 2 to 811, further preferably 2 to 100, more preferably 2-50, even more preferably p is 2 to 30, or 3 to 25, or 4 to 20, which indicates that the residue R⁷ bears p residues of the structure (—X—C(O)-G), with G as defined below. Accordingly, the term “p-valent” does not refer to or restrict the number of optional further substituents other than (—X—C(O)-G) of the residue R⁷, which can be carboxylic groups, carboxylate groups or hydroxyl groups.

According to the invention, the wording “optionally substituted hydrocarbon radical” that may contain optionally one or more specific functional groups and can be substituted by one or more specific functional groups refers to an organyl radical which is linked to one or more further groups via at least one of its carbon atoms, wherein the hydrocarbyl structure of the radical may be interrupted by the specific functional groups as defined to be contained, and one or more hydrogen atoms of the hydrocarbyl group can be substituted by the substituent groups as indicated.

In case of R⁷, for example, one or more hydrogen atoms may be substituted by a hydroxyl group, by a carboxylic group or a carboxylate group.

Further, as the optionally substituted hydrocarbon radical R⁷ specifically may contain one or more groups selected from —O—, —NH—, —C(O)—, —C(S)— and tertiary amino groups

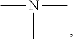

the hydrocarbyl structure of a R⁷ group may be interrupted by these groups or combinations thereof. Accordingly, the residue may contain ester groups, carboxyl groups, amide groups, ether groups, amino groups, carbonyl groups, thione groups, thio carboxylate groups, thio ester groups, carbamate groups, urethane groups, epoxide groups and all other groups as specified for this radical, and combinations thereof.

The hydrocarbyl structure of R⁷, which is p-valent regarding the residues (—X—C(O)-G) in formula (V), is preferably selected from the group consisting of linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 1000 carbon atoms, more particular 1 to 150 carbon atoms.

Preferably, the hydrocarbon structures are linear or branched alkylene groups, or linear or branched alkylene groups interrupted by ether groups, ester groups or both ether and ester groups, in particular branched structures derived from products as obtained by esterification of polyols with mono- or polyhydroxycarboxylic acids with up to 150 carbon atoms, or linear alkylene groups with up to 22 carbon atoms.

More preferably, the p-valent R⁷ radical of formula (V) is selected from alkylene groups, which may be selected from the group consisting of linear, branched and cyclic alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups.

There is no limitation regarding which C-atoms of the hydrocarbyl radicals bear the one or more (—X—C(O)-G) groups attached to R⁷.

Regarding the presence of functional groups optionally contained in R⁷ and optional substituents, it is preferred that R⁷ is derived from glycidyl compounds, glycerol and glycerol derivatives, in particular glycidol, glycerol diglycidyl ether, diglycidyl ether and polyglycerol compounds, or when R⁷ is a linear alkylene group, in particular an alkylene group not bearing further substituents in addition to the (—X—C(O)-G) groups and even more preferred when R⁷ is derived from the condensation product of glycidol, glycerol, glycerol diglycidyl ether, diglycidyl ether and polyglycerol compounds and C8-C24 monohydroxy fatty acids, in particular ricinoleic acid, lesquerolic acid or 12-hydroxyl stearic acid.

In case R⁷ is a dendrimeric structure, it is preferred that it comprises a monocarboxylic acid having 2 to 6 hydroxy groups, whereof at least one, preferably all hydroxyl groups are esterified with monocarboxylic acids bearing 2 to 6 hydroxy groups. Optionally, one, two or more branching cycles may be performed for the addition of further monocarboxylic acids bearing 2 to 6 hydroxy groups via esterification of the hydroxyl groups of the monocarboxylic acids applied in the previous branching cycle.

It is also preferred that the monocarboxylic acids having 2 to 6 hydroxy groups of subsequent branching cycles are linked to each other by estolide chains.

When R⁷ comprises a dendrimeric structure, it is generally preferred that one type of monocarboxylic acids having 2 to 6 hydroxy groups, for example 2,2-bis-(hydroxymethyl) propionic acid), is used to provide the branching structure.

Examples of such R⁷ comprising a dendrimeric structure are as follows:

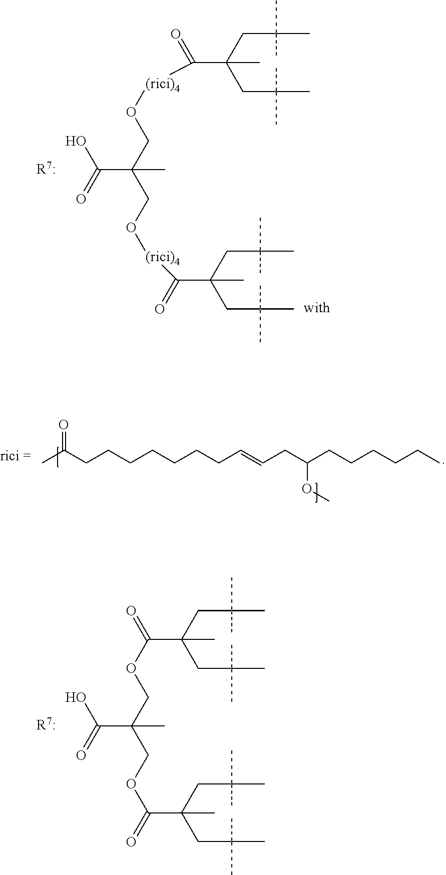

It is clear to the skilled person that usually the groups (—X—C(O)-G) are attached to the radical R⁷ via —X—C(O)— units, in particular —O—C(O)— units, at positions which are substituted by —OH or —NHR¹¹ groups in a parent compound from which R⁷ is derived.

For example, the R⁷ group derived from glycerol is a 1,2,3-propylene radical, wherein “1,2,3” indicates the positions at which the radical is substituted by the (—X—C(O)-G)-groups.

According to the invention, the term “optionally substituted hydrocarbon residue” does not impose any further restrictions on the radicals, and accordingly they are limited by the groups which can be optionally contained or present as substituents, the number of carbon atoms of the residues as specified, and the way they are bonded to other structural moieties of the compound according to the invention as defined by formula (V), formula (VI), formula (VI*) and formula (VII), formula (VIII), formula (VIM), formula (X) or any further formula used to define an embodiment according to the invention.

According to the invention, the group X can be the same or different and is selected from —O—, or —NR¹¹—, wherein R¹¹ is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups

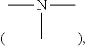

or in formula (V) R¹¹ may form a bond to R⁷ to form a cyclic structure.

Preferred examples of R¹¹ are C1-C10 alkyl groups, in particular methyl, ethyl, n-propyl, iso-propyl, n-butyl, iso-butyl, tert-butyl, n-pentane and n-hexane groups, cyclopentyl groups and cyclohexane groups, C2-C10 alkenyl groups, in particular vinyl groups and allyl groups, and C6-C12 aromatic groups, in particular phenyl groups, tolyl groups, and benzyl groups, wherein each of the named groups may be substituted by hydroxyl groups or halide groups.

According to the invention, the residue G can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, tertiary amino groups

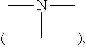

and can be optionally substituted by one or more selected from carboxyl, hydroxyl or halide groups, with the proviso that at least one compound of the radicals G contains at least one moiety of the formula (VI) or (VI*)

—R⁸(—X—C(O)—R⁸)ₘ—X—C(O)—R⁹ (VI),

—R⁸(—X—C(O)—R⁸)ₘ—X—C(O)—R^{9*} (VI*)

wherein X is as defined above,
m=0 to 20, preferably 1 to 20,
R⁸ is independently selected from a divalent optionally substituted straight-chain, cyclic or branched, saturated or unsaturated hydrocarbon radical which have up to 36 carbon atoms,
R⁹ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups

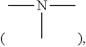

quaternary ammonium groups

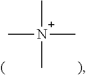

and which can be substituted with one or more substituent groups selected from a carboxyl (—COOH) group, carboxylate anion (—COO⁻) group, a hydroxyl (—OH) group, and a halide (-halogen) group, wherein the radical R⁹ cannot contain an internal carboxy (—COO—) group or (—CON(R′)—, R′ being hydrogen or organic group) amide group, i.e. R⁹ cannot contain a combination of a —C(O)— group and a —O— group or a combination of a —C(O)— group and a —NH— or tertiary amino group, and
with the proviso that in at least one moiety of the formula (VI) R⁹ has at least 2, preferably at least 6 carbon atoms, and that in the same moiety of the formula (VI) at least one R⁸ has at least 6, preferably at least 8 carbon atoms,
R⁹* is independently selected from optionally substituted branched or dendrimeric hydrocarbon radicals which have 1 to 1000 carbon atoms, optionally containing one or more groups selected from —O—, —NH—, —C(O)—, —C(S)—, tertiary amino groups

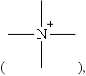

quaternary ammonium groups

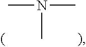

and which can be substituted with carboxyl, hydroxyl, or halide groups, wherein the radical R⁹* is terminated by two or more groups of the general structure

—X—C(O)-T

wherein X is as defined above, and
T is a monovalent straight-chain, cyclic or branched, saturated or unsaturated hydrocarbon radical optionally substituted with carboxyl, hydroxyl, or halide groups with up to 36 carbon atoms,
with the proviso that in at least one moiety of the formula (VI*) R⁹* is terminated by one or more groups T having at least 2, preferably at least 6 carbon atoms, and in the same moiety of the formula (VI*) at least one R⁸ has at least 6, preferably at least 8 carbon atoms.

According to the invention, mandatorily at that at least one of R⁷ and G comprises one or more carboxylate anion (—COO⁻) groups.

Preferably, the group G consists of a group of the formula (VI) only,

or the group G consists of a group of the formula (VI)* only.

According to the invention, R⁸ is independently selected from a divalent optionally substituted straight-chain, cyclic or branched, saturated or unsaturated hydrocarbon radical which have up to 36 carbon atoms. It may thus be a divalent, optionally substituted straight-chain, cyclic or branched, saturated, unsaturated or aromatic hydrocarbon radical, i.e. R⁸ can represent a hydrocarbyl group selected from the group consisting of linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 24 carbon atoms, each optionally containing one or more hydroxy groups.

More preferably, the R⁸ radical is selected from linear alkylene groups and linear alkenylene groups, in particular from linear C6-C24 alkylene such as hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or linear C6-C24 alkenylene groups such as hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group by a terminal C-atom.

There is no limitation regarding at which C-atoms of the hydrocarbyl radicals the adjacent group C(O) group and X group are attached to R⁸.

However, R⁸ is preferably derived from a hydroxycarboxylic acid bearing one or more hydroxylic groups, more preferably from a monohydroxy carboxylic acid, most preferably from C7-C25 fatty acids bearing one hydroxyl group as substituent. Accordingly, R⁸ preferably represents the alkylene or alkenylene chain of such carboxylic acids. For instance, if R⁸ is derived from ricinoleic acid

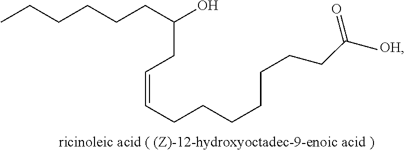 [SMILES: CCCCCCC(O)C/C=C\CCCCCCCC(C)=O]

then R⁸ represents a 1,11-heptadec-8-enyl radical

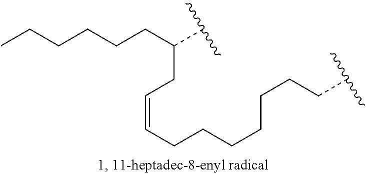

wherein “1,11” indicates the positions in which the radical is attached to the adjacent groups X and C(O).

Preferred examples for R⁸ are the structures derived from a corresponding hydroxyl carboxylic acid by abstraction of the carboxylate group and one OH group, wherein the hydroxyl carboxylic acid is preferably selected from ricinoleic acid, lesquerolic acid, 10-hydroxy octadecanoic acid, 12-hydroxy octadecanoic acid, 14-hydroxy tetradecanoic acid, 10-hydroxy stearic acid, 12-hydroxy stearic acid, or dihydroxy carboxylic acids, in particular 2,2′-di-hydroxymethyl propanoic acid, 9,10-dihydroxy stearic acid, or polyhydroxy carboxylic acids, in particular gluconic acid. Most preferably, R⁸ is derived in the above-stated manner from lesquerolic acid or ricinoleic acid. In both cases the naturally occurring enantiomers of the compounds, i.e. (9Z,12R)-12-hydroxyoctadec-9-enoic acid obtained by saponification or fractional distillation of hydrolysed castor oil, which is the seed oil of the castor plant, and (11Z,14R)-14-hydroxyicos-11-enoic acid as isolated from Paysonia and Physaria species, are particularly preferred. However, the racemates, the S enantiomers as well as the E-configured isomers of the compounds, the racemates, the enantiomers and any possible mixture thereof are also preferred according to the invention.

The number m of the R⁸-containing repeating units (—X—C(O)—R⁸) of the at least one moiety present in a group G of the compound of the general formula (V) is from 0 to 20, preferably from 0 to 15, 0 to 12, 0 to 10, 0 to 8, or from 1 to 20, from 2 to 20, from 3 to 20, from 4 to 20, from 5 to 20, specifically 0, 1, 2, 3, 4, 5, 6, 7, 8, 9, or 10.

R⁹ is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups

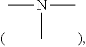

quaternary ammonium groups

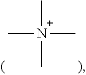

and which can be substituted with one or more substituent groups selected from a carboxyl (—COOH) group, carboxylate anion (—COO⁻) group, a hydroxyl (—OH) group, and a halide (-halogen) group, wherein the radical R⁹ cannot contain an internal carboxy (—COO—) group or (—CON(R′)—, R′ being hydrogen or organic group) amide group, i.e. R⁹ cannot contain a combination of a —C(O)— group and a —O— group or a combination of a —C(O)— group and a —NH— or tertiary amino group.

According to the invention, the radicals R⁹ can be the same or different selected from optionally substituted straight-chain, cyclic or branched, saturated or unsaturated hydrocarbon radicals which have 1 to 36 carbon atoms, and can thus represent a hydrocarbyl group selected from the group consisting of linear, branched or cyclic alkyl groups, linear, branched or cyclic alkenyl groups, linear, branched or cyclic alkynyl groups, linear, branched or cyclic alkaryl groups, linear, branched or cyclic aralkyl groups and linear, branched or cyclic aryl groups, for instance phenyl, benzyl or tolyl, in particular from such groups having 6 to 24 carbon atoms, each optionally containing one or more functional groups as indicated above.

More preferably, the R⁹ radical is selected from linear alkyl groups and linear alkenyl groups, in particular from linear C6-C24 alkyl groups such as hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or linear C6-C24 alkenyl groups such as hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) group or X group by a terminal C-atom,

There is no limitation regarding at which C-atom of the hydrocarbyl radicals the adjacent group C(O) group is attached to R⁹.

However, R⁹ is preferably derived from a carboxylic acid or a hydroxycarboxylic acid bearing one or more hydroxylic groups, more preferably from a carboxylic acid or monohydroxy carboxylic acid, most preferably from C7-C25 fatty acid bearing no hydroxyl group as substituent. Accordingly, R⁹ preferably represents the alkyl or alkenyl chain of such carboxylic acids. For instance, if R⁹ is derived from oleic acid,

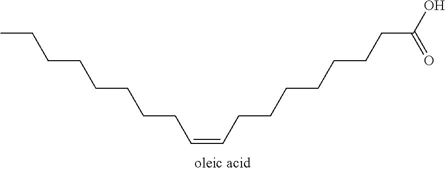 [SMILES: CCCCCCCC/C=C\CCCCCCCC(=O)O]

then R⁹ represents an heptadec-8-enyl radical

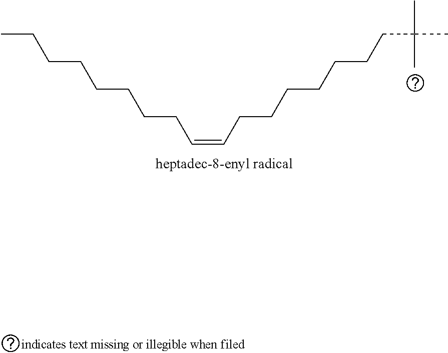

Preferred examples for R⁹ are the structures derived from a corresponding carboxylic acid or hydroxyl carboxylic acid by abstraction of the carboxylate group, wherein the carboxylic acid may be selected from acetic acid, propionic acid, butyric acid, valeric acid, caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, linoleic acid, α-linolenic acid, γ-linolenic acid, nonadecylic acid, arachidic acid, mead's acid, arachidonic acid, heneicosanoic acid, docosanoic acid, tricosylic acid and lignoceric acid, from hydroxyl carboxylic acid such as lesquerolic acid, ricinoleic acid, 10-hydroxy octadecanoic acid, 12-hydroxy octadecanoic acid, 14-hydroxy tetradecanoic acid, 10-hydroxy stearic acid, 12-hydroxy stearic acid, or from dihydroxy carboxylic acids, in particular 2,2′-di-hydroxymethyl propanoic acid, 9,10-dihydroxy stearic acid, or polyhydroxy carboxylic acids, in particular gluconic acid.

Although the radical R⁹ can optionally contain one or more groups selected from —O—, —NH—, —C(O)—, —C(S)—, tertiary amino groups

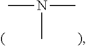

quaternary ammonium groups

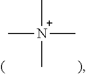

and may be substituted with OH groups or halide groups, the radical R⁹ cannot contain a combination of a —C(O)— group and a —O— group or a combination of a —C(O)— group and a —NH— or tertiary amino group forming an internal carboxylate group, i.e. an internal ester group, or an internal amide group.

According to the invention, it is mandatory that in at least one moiety of the formula (VI) R⁹ has at least 2, preferably at least 6 carbon atoms, in the same moiety of the formula (VI) at least one R⁸ has at least 6, preferably at least 8 carbon atoms.

As stated above, according to the invention R9* is defined as a monovalent group, in order to provide the structural feature of being terminated by at least two groups of the general structure

—X—C(O)-T,

the presence of at least one branching structure is required in the residue R⁹*.

In the case R⁹* is a branched hydrocarbon radical, as the branching structure, the group comprises at least one moiety of the general formula

—B(—O—)_{b},

wherein B is a linear or branched hydrocarbon group having 3-20 carbon atoms, preferably 3-10 carbon atoms, more preferably B is and alkyl radical having 3-10 carbon atoms, and b is 2 or more, preferably 2-6, more preferably 2-4, and wherein
the groups (—O—) linked to the group B on the one side are linked to a C atom on the other side. Therein, the C atom may be of a CH₂ group or of a carbonyl group.

In the case R⁹* is a dendrimeric hydrocarbon radical, the group comprises at least one moiety of the general formula

—B(—O—)_{b},

wherein B and b are as defined above, and the groups (—O—) linked to the group B on the one side are linked to a C atom on the other side, and
to at least one further moiety acting as branching structure of the general formula

—C(O)—B(—O—)_{b},

wherein B and b are as defined above, and the groups (—O—) linked to the group B on the one side are linked to a C atom on the other side, wherein the C atom may be of a CH₂ group or of a carbonyl group. The term dendrimeric hydrocarbon structure thus refers to a branched structure containing at least two consecutive branching structures.

The group R^{9*} may also comprise two or more branching structures which are linked by an estolide-chain structure, i.e. wherein two or more branching structures are linked by a linear hydrocarbon chain containing at least two internal ester groups, preferably a linear hydrocarbon chain obtained by esterification of two or more C2-C24 hydroxycarboxylic acids.

Each group T constitutes one of at least two terminal groups of a R^{9*} group and is typically derived from a fatty acid. Accordingly, the group T is preferably a linear saturated or monounsaturated hydrocarbon radical having 2 to 24 carbon atoms.

The group T is preferably linked to an (—O—) group of a branching structure of the general formula —B(—O—)_{b}, or —C(O)—B(—O—)_{b} via a carbonyl group or via an estolide chain.

By the presence of the above-described branching structures, R^{9*} adopts a branched or even dendrimeric structure.

The following structure is an example of R^{9*} being a branched hydrocarbon radical as defined above:

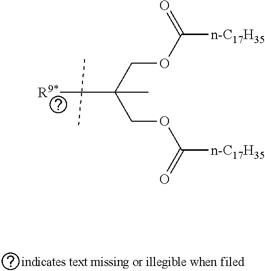

Therein, the branching structure of the general formula B(—O—)_{b} is derived from 2,2′-dihydroxymethylpropionic acid, and the group T is a n-heptadecanyl group linked to the branching structure. It is derived from stearic acid and linked to the group B by a —C(O)—O-unit. Accordingly, the structure is terminated by two groups of the general structure —X—(CO)-T and contains a branching structure of the formula —B(—O—)₂.

An example of a group from which a branched group R^{9*} may be derived is displayed below:

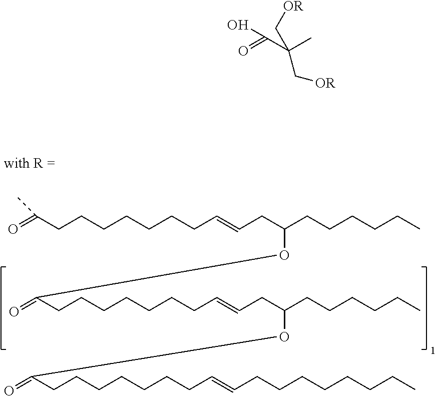

In the corresponding group R^{9*}, the branching structure is the same as in the previous structure, while the two terminal groups T, which are n-heptadec-9-enyl groups derived from oleic acid, are attached to the branching structure via a ricinoleic-acid derived estolide chain structure.

Another example of a group R^{9*} according to the invention being a dendrimeric hydrocarbon is displayed below:

Therein, the branching structure —B(—O—)_{b} is directly followed by two further branching structures —(C(O)—B(—O—)_{b}, resulting in a further increase of terminating groups of the general structure —X—C(O)-T:

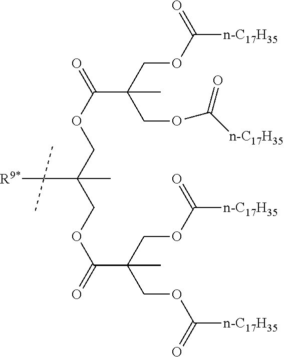

Therein, the branching structures are derived from 2,2′-dihydroxymethylpropionic acid, and the terminal groups are based on stearic acid.

It is also within the scope of the invention as defined above that in the branched or dendrimeric group R⁹*, the terminal groups —X—C(O)-T are not linked directly to the groups B of a branching structure, but are linked to the (—O—) groups of the branching structures by hydrocarbon groups such as optionally substituted or heteroatom-group-containing alkylenes or alkenylenes, preferably n-alkylenes having 2 to 10 carbon atoms, poly(alkylene oxide) groups such as poly (ethylene oxide) or poly (propylene oxide) groups, or in particular by oligo- or polyester groups, i.e. by estolide chains.

In the following example, the stearic acid-based groups —X—C(O)-T are linked to the branching structures by an estolide chain:

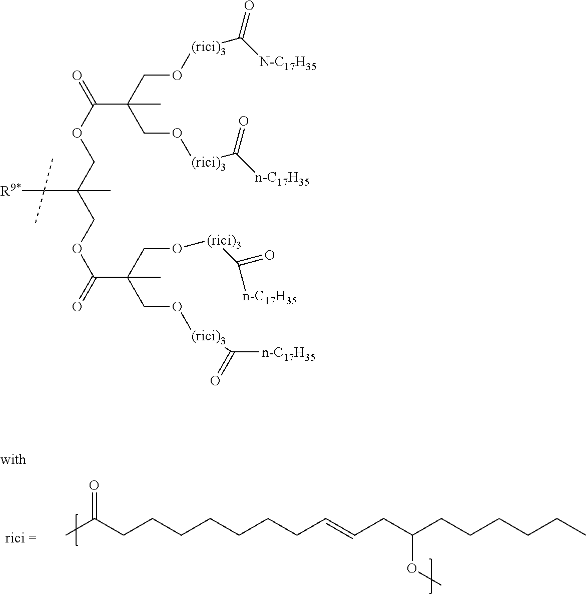

As already indicated above, in the same manner it is also within the scope of the invention as defined above that in the dendrimeric group R^{9*}, the one or more branching elements of the structure —(C(O)—B(—O—)_{b} are not directly attached to a branching element of the structure —B(—O—)_{b} or —(C(O)—B(—O—)_{b}, but linked via hydrocarbon groups such as optionally substituted or heteroatom-group-containing alkylenes or alkenylenes, preferably n-alkylenes having 2 to 10 carbon atoms, poly(alkylene oxide) groups such as poly (ethylene oxide) or poly (propylene oxide) groups, or in particular by oligo- or polyester groups, i.e. by estolide chains.

In the following example, the branching structures are linked by estolide chains:

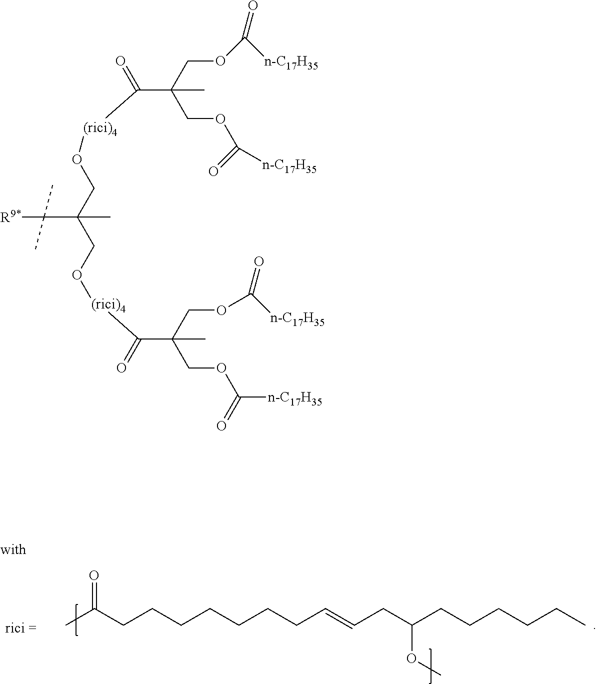

It is further mandatory that at least one R⁷ and G in the carboxylate anion (COO⁻) group-comprising anion of the formula (V) comprises one or more carboxylate anion (—COO⁻) groups, preferably one, two or three carboxylate groups.

The structure of the anions of the formula (VII)

R⁷(—C(O)—X—Y)_{q} (VII),

is defined as follows:

R⁷ and X are as defined above for formula (V), except that in the formula (VII), R⁷ is q-valent regarding the residue (—C(O)—X—Y). Otherwise, all selections indicated as being preferred for R⁷ and X in formula (V) are likewise preferred for R⁷ and X in formula (VII).

Y can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups

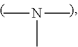

and can be substituted by one or more substituent groups selected from a carboxyl (—COOH) group, a carboxylate anion (—COO⁻) group and a hydroxyl group,
with the proviso that at least one of the radicals Y contains at least one moiety of the formula (VIII) or (VIII*):

—R⁸(—C(O)—X—R⁸)ₘ—C(O)—X—R⁹ (VIII)

—R⁸(—C(O)—X—R⁸)ₘ—C(O)—X—R^{9*} (VIII*)

wherein X, m, R⁸, and R⁹ in formula (VIII) are each as defined above for formula (VI), and. X, m, R⁸, and R^{9*} in formula (VIII*) are each as defined above for formula (VI*),

Preferably, the group Y consists of a group of the formula (VIII) only,

or of a group of the formula (VIII*) only.

It is further preferred that the minimum chain length of at least one R⁸ in the formulas (VIII) and (VIII*) is 8 carbon atoms, more preferably the chain length of all groups R⁸ in the formulas (VIII) and (VIII*) is at least 8 carbon atoms, and it is also preferred that the minimum chain length of at least one R⁹ in the formulas (VIII) and (VIII*) is 8 carbon atoms, more preferably the chain length of all groups R⁹ in the formulas (VIII) and (VIII*) of the anion of the organic ammonium salt is at least 8 carbon atoms.

It is mandatory that at least one of R⁷ and Y in formula (VII) comprises one or more carboxylate anion (—COO—) groups, preferably one or two or three.

In the anions of the formula (VII),

q is 1 to 55, preferably 1 to 40, more preferably 2 to 25, even more preferably 2 to 15, and most preferably 2 to 4.

The structure of the anions of the formula (X)

R⁷(—C(O)—X—R⁸—COO⁻)_{q} (X)

is defined as follows:
R⁷ and X are as defined above for formula (VII), except that in the formula (X), R⁷ is q-valent regarding the residue (—C(O)—X—R⁸—COO⁻), and R⁸ and q are as defined above in formula (VIII). Preferably, q is in the range of 1-3, specifically 1, 2 or 3.

According to the formulas (V) and (VII) and the groups contained in said structures, it is understood that the anions of the organic ammonium salts of the invention may comprise branched or dendrimeric branching structures in the central moiety R⁷, and they may comprise branched or dendrimeric branching structures at an internal position or adjacent to the terminal groups of the groups R9* of the formulas (VI*) and (VIII*). In general, it is within the scope that the only groups Y and G comprise branching structures, and it is also within the scope of the invention that both R⁷ and G or R⁷ and Y comprise branching structures, as long as the requirements regarding the presence of estolide moieties are fulfilled.

In a preferred embodiment of the invention, the organic ammonium-group-comprising cation of the organic ammonium salt according to the invention is selected from cations of the formula (I):

R¹(—F)ₓ (I),

wherein
x is 1 to 50, preferably 2 to 50,
R¹ is selected from x-valent, optionally substituted hydrocarbon radicals which have up to 1000 carbon atoms, preferred 2 to 300 carbon atoms, more preferred 3 to 200 carbon atoms, even more preferred 3 to and 150 carbon atoms, specifically 3 to 50 carbon atoms, more specifically 3 to 20 carbon atoms, and may contain optionally one or more groups selected from —O—,
—NH—, —C(O)—, —C(S)—, tertiary amino groups

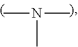

and R¹ can be substituted by one or more groups selected from a carboxyl (—COOH) group, a carboxylate anion (—COO⁻) group, a hydroxyl (—OH) group and a halide (-halogen) group, and
F can be the same or different and is represented by the general formula (II)

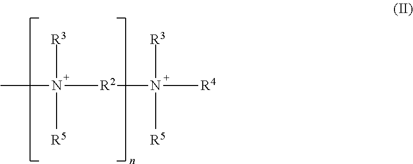

wherein the groups F bind to a carbon atom of R¹, and
n is independently 0 to 1000,
R² can be the same or different and is selected from divalent optionally substituted hydrocarbon radicals which have up to 1000 carbon atoms, and optionally contain one or more groups selected from —O—, —NH—, —C(O)—, —C(S)—, tertiary amino groups

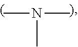

and R² can be substituted with one or more groups selected from OH groups and halide groups, and
R³, R⁴, R⁵ can be the same or different and are selected from hydrogen and optionally substituted straight-chain, cyclic or branched, saturated, unsaturated or aromatic hydrocarbon radicals which have up to 1000 carbon atoms, which optionally contain one or more groups selected from —O—, —NH—, —C(O)—, —C(S)—, tertiary amino groups

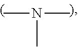

quaternary ammonium groups

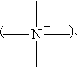

and which can be substituted with one or more groups selected from OH groups and halide groups,
wherein if R³, R⁴, R⁵ are not hydrogen they each bind with a carbon atom to the nitrogen atom.

According to the invention, the residue R¹ is x-valent, wherein x is 1 to 50, preferably 2 to 50, which indicates that the residue R¹ bears x residues F as defined by the general formula (II). Accordingly, the term “x-valent” does not refer to or restrict the number of optional further substituents other than F of the residue R¹, which can be hydroxyl groups and halide groups.

In R¹ one or more hydrogen atoms may be substituted by a hydroxyl group or by an halide substituent, i.e. by a fluoro, chloro, bromo or iodo substituent.

Further, as the optionally substituted hydrocarbon radical R¹ specifically may contain one or more groups selected from —O—, —NH—, —C(O)—, —C(S)— and tertiary amino groups

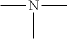

the hydrocarbyl structure of a R¹ group may be interrupted by these groups or combinations thereof. Accordingly, the residue may contain ester groups, carboxyl groups, amide groups, ether groups, amino groups, carbonyl groups, thione groups, thio carboxylate groups, thio ester groups, carbamate groups, urethane groups, epoxide groups and all other groups as specified for this radical, and combinations thereof. The same principle applies to the optionally substituted hydrocarbon radicals R², R³, R⁴, R⁵. However, the above-mentioned groups may not be combined in such way that moieties of the formulas (III) or (IV) as defined above are formed.

The hydrocarbyl structure of R¹, which is x-valent regarding the residues F, is preferably selected from the group consisting of linear, branched or cyclic alkyl or alkylene groups, linear, branched or cyclic alkenyl or alkenylene groups, linear, branched or cyclic alkynyl or alkynylene groups, linear, branched or cyclic alkaryl or alkarylene groups, linear, branched or cyclic aralkyl or aralkylene groups and linear, branched or cyclic aryl or arylene groups, for instance phenyl or phenylene, benzyl or benzylene or tolyl or tolylene groups, in particular from such groups having 1 to 30 carbon atoms.

More preferably, the x-valent R¹ radical is selected from alkyl or alkylene groups, which may be selected from the group consisting of linear, branched and cyclic alkyl or alkylene groups or groups combining linear and cyclic alkyl or alkylene structures, or groups combining branched and cyclic structures, in particular from linear C1-C22 alkyl groups such as methyl and methylene, ethyl and ethylene, n-propyl and n-propylene, n-butyl and n-butylene, n-pentyl and n-pentylene, n-hexyl and n-hexylene, n-heptyl and n-heptylene or n-octyl and n-octylene groups, branched C1-C22 alkyl and alkylene groups such as iso-propyl and iso-propylene, iso-butyl and iso-butylene, tert-butyl and tert-butylene, iso-pentyl and iso-butylene, tert-pentyl and tert-pentylene, neo-pentyl and neo-pentylene, and 2-ethylhexyl and 2-ethylhexylene groups, and from cyclic C3-C22 alkyl groups such as cyclopropyl or cyclopropylene, cyclobutyl and cyclobutylene, cyclopentyl and cyclopentylene, cyclohexyl and cyclohexylene, and cycloheptyl or cycloheptylene groups.

In case x is >1, there is no limitation regarding at which C-atoms of the hydrocarbyl radicals the groups F are bonded to R¹. Regarding the presence of functional groups optionally contained in R¹ and optional substituents, it is preferred that R¹ is derived from glycidyl compounds, glycerol and glycerol derivatives, in particular glycidol, glycerol, glycerol diglycidyl ether, diglycidyl ether and polyglycerol compounds, or when R¹ is a linear alkylene group, in particular an alkylene group not bearing further substituents in addition to the F groups.

According to the invention, it is particularly preferred when R¹ is derived from glycerol diglycidyl ether, which means that R² is formed by opening of the epoxide rings of glycerol diglycidyl ether by N atoms then forming the quaternary N atoms adjacent to the R¹ group in the compounds according to the invention. In the same manner, it is preferred when R¹ is derived from diglycidyl ether, diglycerol diglycidyl ether, triglycerol diglycidyl ether, polyglycerols terminated with glycidyl units, and poly(alkylene oxide) compounds terminated with glycidyl units, in particular poly(ethylene oxide)s terminated with glycidyl units, poly(propylene oxide)s terminated with glycidyl units, and poly(butylene oxide)s terminated with glycidyl units.

It is also preferred when R¹ is formed from compounds obtained by esterification of polyols, in particular diol compounds such as α,ω-diols or α,ω-dihydroxypolyethers, more particular dihydroxy-terminated poly(ethylene oxide), dihydroxy-terminated poly(propylene oxide) or dihydroxy-terminated poly(butylene oxide) with ω-halocarboxylic acids, in particular ω-chloro acetic acid or ω-chloropropanoic acid. Latter compounds form R¹ by substitution of the chloro substituents by the N-atoms of the F groups adjacent to the R¹ group.

According to this, it is preferred when R¹ is a C3-C50 alkylene group containing one or more internal ether or ester groups, and it is particularly preferred when R¹ is such alkylene group bearing hydroxyl substituents.

It is most preferred when R¹ is a linear C1-C8 alkylene group without further substituents or functional groups, or when R² is a linear C3 to C50 alkylene group derived from diglycidyl ether, glycerol diglycidyl ether, diglycerol diglycidyl ether, diethylene glycol diglycidyl ether, or ethylene glycol diglycidyl ether with 3 to 10 (ethylene oxide) repeating units.

The residues R², R³, R⁴, and R⁵ can be optionally substituted straight-chain, cyclic or branched, saturated, unsaturated or aromatic hydrocarbon radicals, wherein R² represents divalent radicals, while R³, R⁴ and R⁵ are monovalent radicals. In case of R² the term “divalent” refers to R² being bonded to two quaternary N atoms according to formula (II), but does not limit the presence of further other substituents as defined for R².

The radicals R³, R⁴ and R⁵ are monovalent radicals which can be the same or different and are selected from hydrogen and optionally substituted straight-chain, cyclic or branched, saturated, unsaturated or aromatic hydrocarbon radicals which have up to 1000 carbon atoms, and can thus represent linear, i.e. straight-chained, cyclic or branched alkyl groups, linear, cyclic or branched alkenyl groups, linear, cyclic or branched alkynyl groups, linear, cyclic or branched alkaryl groups, linear, cyclic or branched aralkyl groups and aryl groups, for instance phenyl, benzyl or tolyl groups, in particular groups having 1 to 30 carbon atoms, and optionally the aforementioned groups may be substituted with OH or halide groups, and may optionally contain one or more groups selected from —O—, —NH®, —C(O)—, —C(S)—, tertiary amino groups

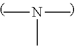

and quaternary ammonium groups

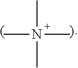

According to the invention, the aforementioned groups may not be combined in such way that moieties of the formulas (III) or (IV) as defined above are present in any of the groups R¹, R², R³, R⁴ and R⁵.

Preferably, the radicals R³, R⁴, and R⁵ are selected from alkyl groups, which may be selected from the group consisting of linear, branched and cyclic alkyl groups or groups combining linear and cyclic alkyl motifs, or structures combining branched and cyclic structures, in particular from linear C1-C22 alkyl groups such as methyl, ethyl, n-propyl, n-butyl, n-pentyl, n-hexyl, n-hexyl, n-heptyl or n-octyl groups, branched C1-C22 alkyl groups such as iso-propyl, iso-butyl, tert-butyl, iso-pentyl, tert-pentyl, neo-pentyl and 2-ethylhexyl groups, and from cyclic C3-C22 alkyl groups such as cyclopropyl, cyclobutyl, cyclopentyl, cyclohexyl and cycloheptyl groups, more preferably the radicals R³, R⁴, and R⁵ are selected from methyl, ethyl, isopropyl, tert-butyl, cyclopentyl or cyclohexyl groups, most preferably from methyl.

The radicals R² according to the invention can be the same or different and is selected from divalent optionally substituted hydrocarbon radicals which have up to 1000 carbon atoms, and optionally contain one or more groups selected from —O—, —NH—, —C(O)—, —C(S)—, tertiary amino groups

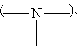

and can be substituted with one or more groups selected from OH groups and halide groups, and are preferably selected from the group consisting of linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 100 carbon atoms, each optionally containing one or more functional groups as indicated above.

More preferably, the R² radical is selected from an alkylene groups, which may be selected from the group consisting of linear, branched and cyclic alkylene groups or groups combining linear and cyclic alkylene structures, or groups combining branched and cyclic structures, in particular from linear C1-C50 alkyene groups such as methylene, ethylene, n-propylene, n-butylene, n-pentylene, n-hexylene, n-heptylene or n-octylene groups, branched C4-C50 alkylene groups such as iso-propylene, iso-butylene, tert-butylene, tert-pentylene, neo-pentylene, 2-ethylhexylene groups, and from cyclic C3-C22 alkyl groups such as cyclopropylene, cyclobutylene, cyclopentylene, cyclohexylene, and cycloheptylene groups.

There is no limitation regarding at which C-atoms of the hydrocarbyl radicals the quaternary N atoms are bonded to R².

According to the invention, n is independently 0-1000, preferably 0 to 500, more preferably 0-250, even more preferably 0-50, even further preferably 0-25 or 0-10.

It is also preferred that n is at least 1, 2, 3, 4, 5, 6, 7, 8 or 9

Regarding the presence of functional groups optionally contained in R² and optional substituents, it is preferred that R² is derived from glycidyl compounds, glycerol and glycerol derivatives, in particular glycidol, glycerol diglycidyl ether, diglycidyl ether and polyglycerol compounds, or when R² is a linear alkylene group, in particular an alkylene group not bearing further substituents in addition to the quaternary N atoms.

As stated above, it is particularly preferred when R² is derived from glycerol diglycidyl ether, which means that R² is formed by opening of the epoxide rings of glycerol diglycidyl ether by N atoms then forming the quaternary N atoms adjacent to the R² group in the compounds according to the invention. In the same manner, it is preferred when R² is derived from diglycidyl ether, diglycerol diglycidyl ether, triglycerol diglycidyl ether, polyglycerols terminated with glycidyl units, and poly(alkylene oxide) compounds terminated with glycidyl units, in particular poly(ethylene oxide)s terminated with glycidyl units, poly(propylene oxide)s terminated with glycidyl units, and poly(butylene oxide)s terminated with glycidyl units.

It is also preferred when R² is formed from compounds obtained by esterification of diol compounds such as α,ω-diols or α,ω-dihydroxypolyethers, in particular dihydroxy-terminated poly(ethylene oxide), dihydroxy-terminated poly(propylene oxide) or dihydroxy-terminated poly(butylene oxide) with ω-halocarboxylic acids, in particular ω-chloro acetic acid or ω-chloropropanoic acid. Latter compounds form R² by substitution of the chloro substituents by the N-atoms adjacent to the R² group.

According to the invention, it is also preferred when R² is a C3-C50 alkylene group containing one or more internal ether or ester groups, and it is particularly preferred when R² is such alkylene group bearing hydroxyl substituents.

It is most preferred when R² is a linear C1-C8 alkylene group without further substituents or functional groups, or when R² is a linear C3 to C50 alkylene group derived from diglycidyl ether, glycerol diglycidyl ether, diglycerol diglycidyl ether, diethylene glycol diglycidyl ether, or ethylene glycol diglycidyl ether with 3 to 10 (ethylene oxide) repeating units.

Preferred examples for the residue R¹ are C3-C18 hydroxy-group-substituted polyether radicals, in particular glycerol-based polyether radicals, and C1-C8 linear alkyl or alkylene groups. Herein, the term polyether comprises in particular poly(alkylene oxide)-derived compounds, wherein the alkylene groups of the repeating units are independently selected from C1-C8 alkylenes.

Preferred examples for the residue R² are linear C1-C8 alkylene radicals, more preferably ethylene, propylene, butylene, pentylene, hexylene and heptylene, most preferably propylene and hexylene.

Preferred examples for the residues R³, R⁴ and R⁵ are linear C1-C8 alkyl groups most preferably R³, R⁴ and R⁵ are methyl groups.

In a further preferred embodiment of the invention, the organic ammonium-group-comprising cation of the organic ammonium salt according to the invention is selected from the group of
a. mono and polyquaternium cations,
b. basic amino acid cations,
c. mono and poly tertiary amine based cations
d. mono and poly secondary amine based cations
e. mono and poly primary amine based cations.

According to the invention, a mono quaternium cation is a cation comprising one quaternary ammonium cation, of the structure NR₄⁺, wherein R is independently selected from alkyl, alkenyl groups and aryl groups, while polyquaternium cations are polycationic cations characterized by the presence of two or more quaternary ammonium cations as described before.

According to the invention, basic amino acid cations are cations formed from amino acids having basic side chains at neutral pH, for example lysine, arginine, histidine, by protonation and/or alkylation of a second amino group of the zwitterionic amino acid.

The term also comprises cations derived from esters and amides of amino acids by protonation or alkylation of one or more amino groups.

According to the invention, a mono tertiary amine cation is a cation comprising one tertiary ammonium cation, of the structure NHR₃⁺, wherein R is independently selected from alkyl, alkenyl groups and aryl groups, while poly tertiary amine cations are polycationic cations characterized by the presence of two or more tertiary ammonium cations as described before.

According to the invention, a mono secondary amine cation is a cation comprising one secondary ammonium cation, of the structure NH₂R₂⁺, wherein R is independently selected from alkyl, alkenyl groups and aryl groups, while poly secondary amine cations are polycationic cations characterized by the presence of two or more secondary ammonium cations as described before.

According to the invention, a mono primary amine cation is a cation comprising one primary ammonium cation, of the structure NH₃R⁺, wherein R is independently selected from alkyl, alkenyl groups and aryl groups, while poly tertiary amine cations are polycationic cations characterized by the presence of two or more tertiary ammonium cations as described before.

In another preferred embodiment of the invention, the organic ammonium-group-comprising cation of the organic ammonium salt according to the invention has at least 6, preferably at least 10 carbon atoms.

In still another preferred embodiment of the invention, the organic ammonium-group-comprising cation of the organic ammonium salt according to the invention has the formula (I), wherein x is 2 and R¹ carries a carboxylate anion group, and the total charge of the cation is +1.

It is preferred that the carboxylate anion group of R¹ is the only carboxylate anion group of the cation, and n is 0 in both groups F of the cation.

Examples of such cations are cations obtained from arginine, histidine or lysine by protonation or alkylation, displaying two positively charged ammonium moieties and a carboxylate anion moiety.

In a preferred embodiment of the invention, the carboxylate anion (COO⁻) group-comprising anion or anions of the organic ammonium salt according to the invention are selected from a group consisting of polymeric fatty acid carboxylates of the type

R⁷[(—C(O)—X—R⁸)ₘ₊₁—C(O)—X—R⁹]_{q} or

R⁷[(X—C(O)—R⁸)ₘ₊₁—X—C(O)—R⁹]ₚ,

wherein X, R⁷, R⁸, R⁹, m, p and q are as defined above for the formulas (V), (VI), (VII) and (VIII) and wherein either R⁷ or at least one of R⁹, or both R⁷ and at least one of R⁹ bear one or more carboxylate groups,
preferably with X=O,
in particular
linear polymeric fatty acid carboxylates of the type

⁻O—C(O)—R⁷(—X—C(O)—R⁸)ₘ₊₁—X—C(O)—R⁹, preferably

⁻O—C(O)—R⁷—(O—C(O)—R⁸)ₘ₊₁—O—C(O)—R⁹,

wherein R⁹ is selected from monovalent optionally substituted straight-chain, cyclic or branched, saturated or unsaturated hydrocarbon radicals which have up to 36 carbon atoms, i.e. derived from linear poly fatty acid structures,
such as

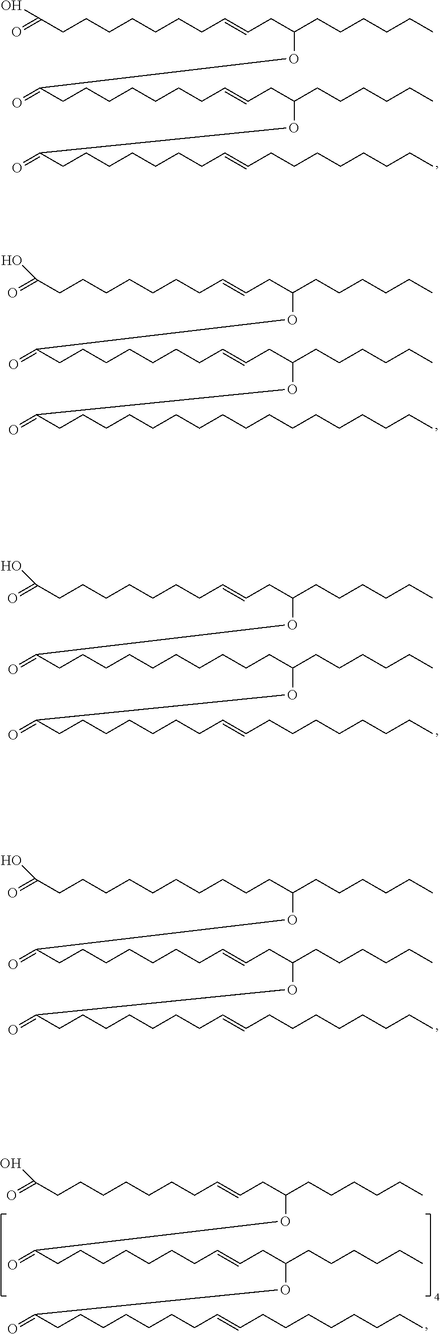

by deprotonation,
   branched linear polymeric fatty acid carboxylates,
   i.e. derived from branched poly fatty acid structures, such as

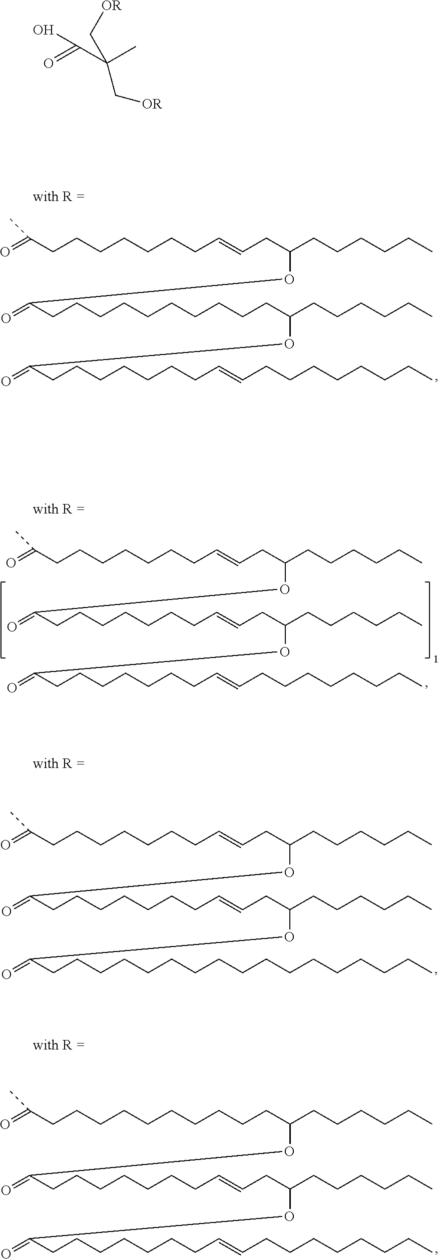

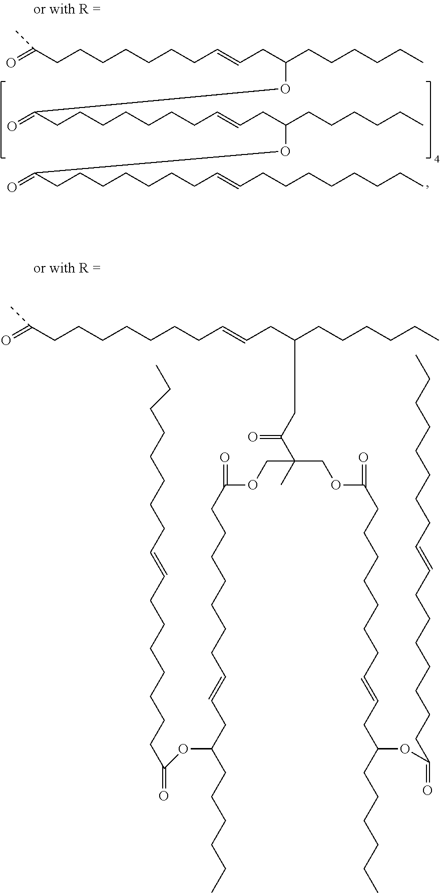

or branched linear polymeric fatty acid carboxylates derived from partial esters of polyfunctional carboxylic acids, in particular of the dicarboxylic acids succinic acid and maleic acid, with castor oil or lesquerella oil, such as

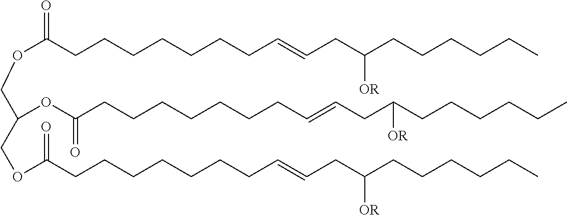

with one or two R=

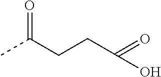

and the remaining two or one R groups=

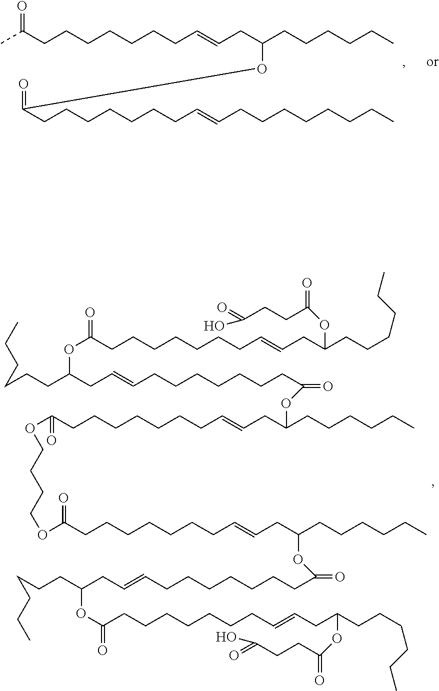

dendritic polymeric fatty acid carboxylates,
i.e. derived from dendritic poly fatty acid structures,
such as

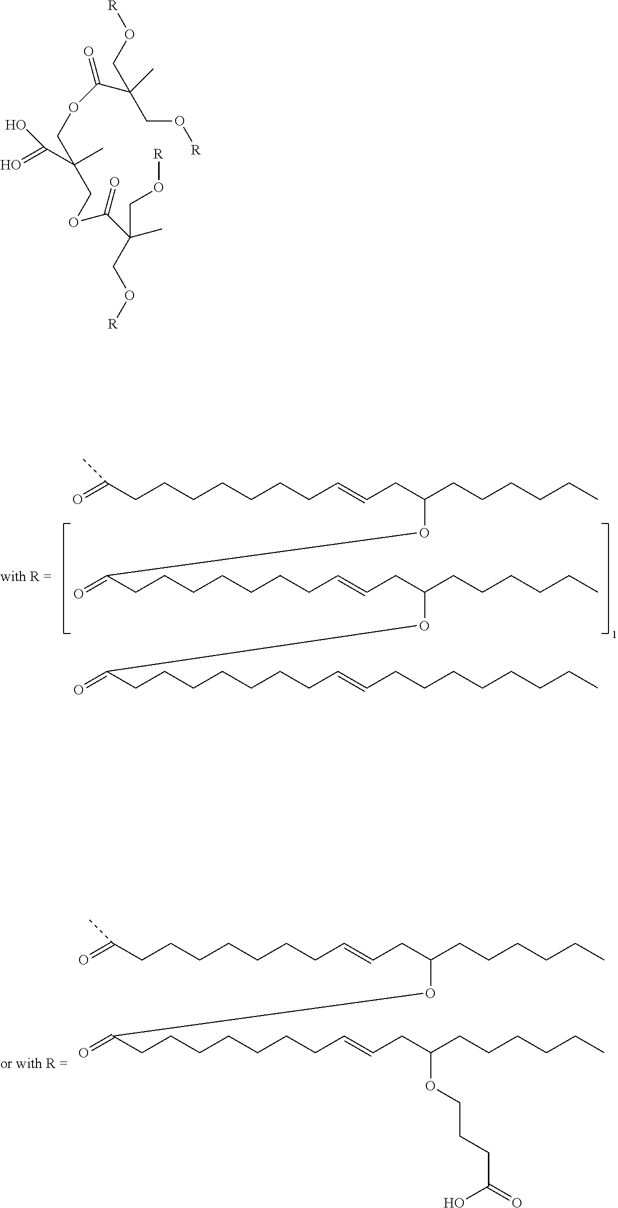

or of the types

R⁷[(—C(O)—X—R⁸)ₘ₊₁—C(O)—X—R⁹C(O)O⁻]_{q}

or (⁻OC(O))_{q-1}—R⁷—(C(O)—X—R⁸)ₘ₊₁—C(O)—X—R⁹C(O)O⁻

wherein X, R⁷, R⁸, R⁹, m, p and q are as defined above for the formulas (V), (VI), (VII) and (VIII), such as

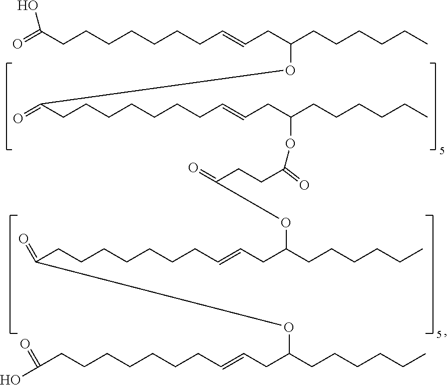

and
wherein the carboxylate anion (COO⁻) group-comprising anion or anions of these types, in particular of the types

R⁷[(—C(O)—X—R⁸)ₘ₊₁—C(O)—X—R⁹C(O)O⁻]_{q}

or (⁻OC(O))_{q-1}—R⁷—(C(O)—X—R⁸)ₘ₊₁—C(O)—X—R⁹C(O)O⁻

are preferably mono- to pentacontavalent, more preferably mono- to decavalent, even more preferably mono- to pentavalent, most preferably pentavalent, tetravalent, trivalent, divalent or monovalent anions.

In another preferred embodiment of the invention, the carboxylate anion (COO⁻) group-comprising anion or anions of the organic ammonium salt according to the invention contain at least one moiety of the general formula (VIa)

(—X—C(O)—R⁸)ₘ—X—C(O)—R⁹ (VIa),

or of the general formula (VIIIa)

(—C(O)—X—R⁸)ₘ—C(O)—X—R⁹ (VIIIa)

wherein X and R⁸ and m are as defined above, and
R⁹ is independently selected from optionally substituted straight-chain, cyclic or branched, saturated or unsaturated hydrocarbon radicals which have 1 to 36 carbon atoms, optionally containing one or more groups selected from —O—, —NH—, —C(O)—, —C(S)—, and which can be substituted with OH groups, carboxylate groups or halide groups.

The radicals R⁹ can be the same or different and are selected from optionally substituted straight-chain, cyclic or branched, saturated or unsaturated hydrocarbon radicals which have 1 to 36 carbon atoms, and preferably represent a hydrocarbyl group selected from the group consisting of linear, branched or cyclic alkyl groups, linear, branched or cyclic alkenyl groups, linear, branched or cyclic alkynyl groups, linear, branched or cyclic alkaryl groups, linear, branched or cyclic aralkyl groups and linear, branched or cyclic aryl groups, for instance phenyl, benzyl or tolyl, more preferably such groups having 6 to 24 carbon atoms, each optionally containing one or more functional groups as indicated above.

In still another preferred embodiment of the invention, at least one group R⁹ of the organic ammonium salt according to the invention is selected from linear alkyl groups and linear alkenyl groups, in particular from linear C6-C24 alkyl groups such as hexyl, heptyl, octyl, nonyl, decyl, undecyl, dodecyl, tridecyl, tetradecyl, pentadecyl, hexadecyl, heptadecyl, octadecylene, nonadecyl, eicosyl, henicosyl, doicosyl, tricosyl, and tetraicosyl, or linear C6-C24 alkenyl groups such as hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent X group or —C(O)— group by a terminal C-atom.

Preferably, all terminal groups R⁹ of a carboxylate anion (COO—) group-comprising anion are selected from linear alkyl or linear alkenyl groups as described above, and more preferably all R⁹ groups are selected from C6-C24 alkyl groups, in particular C14-C22 alkyl groups.

In a further preferred embodiment of the invention, at least one group R⁹ of the organic ammonium salt according to the invention is derived from a carboxylic acid or a hydroxycarboxylic acid bearing one or more hydroxylic groups, more preferably from a carboxylic acid or monohydroxy carboxylic acid, most preferably from a C7-C25 fatty acid bearing no hydroxyl group as substituent.

In particular, it is preferred that at least one group R⁹ of the organic ammonium salt according to the invention is derived from a hydroxyl carboxylic acid selected from ricinoleic acid, lesquerolic acid, 10-hydroxy octadecanoic acid, 12-hydroxy octadecanoic acid, 14-hydroxy tetradecanoic acid, 10-hydroxy stearic acid, 12-hydroxy stearic acid, derived from a dihydroxy carboxylic acid selected from 2,2′-di-hydroxymethyl propanoic acid, 9,10-dihydroxy stearic acid, derived from gluconic acid, or derived from carboxylic acids selected from acetic acid, propionic acid, butyric acid, valeric acid, caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, oleic acid, linoleic acid, α-linolenic acid, γ-linolenic acid, nonadecylic acid, arachidic acid, mead's acid, arachidonic acid, heneicosanoic acid, docosanoic acid, tricosylic acid and lignoceric acid,

most preferably R⁹ is selected from ricinoleic acid, lesquerolic acid, 10-hydroxy stearic acid, 12-hydroxy stearic acid, palmitic acid, margaric acid, stearic acid, linoleic acid, α-linolenic acid, γ-linolenic acid, oleic acid, nonadecylic acid, and arachidic acid.

Even more preferably, all groups R⁹ are derived from the aforementioned carboxylic acids and monohydroxy acids, most preferably from ricinoleic acid, lesquerolic acid, oleic acid and stearic acid.

In an even further preferred embodiment of the invention, at least one group R⁹ of the organic ammonium salt according to the invention represents the alkyl or alkenyl chain of a carboxylic acid or hydroxyl carboxylic acid obtained by abstraction of the carboxylate group, and wherein preferably the carboxylic acid is selected from acetic acid, propionic acid, butyric acid, valeric acid, caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, linoleic acid, α-linolenic acid, γ-linolenic acid, oleic acid, nonadecylic acid, arachidic acid, mead's acid, arachidonic acid, heneicosanoic acid, docosanoic acid, tricosylic acid and lignoceric acid, from hydroxyl carboxylic acid such as lesquerolic acid, ricinoleic acid, 10-hydroxy octadecanoic acid, 12-hydroxy octadecanoic acid, 14-hydroxy tetradecanoic acid, 10-hydroxy stearic acid, 12-hydroxy stearic acid, or from dihydroxy carboxylic acids, in particular 2,2′-di-hydroxymethyl propanoic acid, 9,10-dihydroxy stearic acid, or polyhydroxy carboxylic acids, in particular gluconic acid,

more preferably at least one R⁹ radical is derived from palmitic acid, margaric acid, stearic acid, linoleic acid, α-linolenic acid, γ-linolenic acid, oleic acid, nonadecylic acid, arachidic acid, 10-hydroxy stearic acid, 12-hydroxy stearic acid, ricinoleic acid, lesquerolic acid or from 2,2′-di-hydroxymethyl propanoic acid,
and most preferably at least one R⁹ radical is derived from oleic acid, stearic acid, lesquerolic acid and ricinoleic acid.

In a preferred embodiment of the invention, in the carboxylate anion (COO—) group-comprising anion or anions of the general formulas (V), (VII) and (X) of the organic ammonium salt according to the invention,

### X=O,

R⁸ is independently selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group or O group by a terminal C-atom,
R⁹ is independently selected from optionally hydroxyl-substituted hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, doicosyl, tricosyl, and tetraicosyl, or hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, henicosenyl, doicosenyl, tricosenyl, and tetraicosenyl, wherein the groups are most preferably bonded to the adjacent C(O) or O group by a terminal C-atom, and
m is 0-10, preferably 1-10, more preferably 1, 2, 3, 4 or 5,

In another preferred embodiment of the invention,

### X=O,

at least one group R⁸ in the carboxylate anion (COO—) group-comprising anion or anions of the organic ammonium salt according to the invention is selected from hexadecylene, heptadecylene, octadecylene, nonadecylene, eicosylene, hexadecenylene, heptadecenylene, octadecenylene, nonadecenylene, eicosenylene, and
at least one R⁹ is selected from hexadecyl, heptadecyl, octadecyl, nonadecyl, eicosyl, hexadecenyl, heptadecenyl, octadecenyl, nonadecenyl, eicosenyl,
and m is 1, 2, 3, 4 or 5,
preferably
at least one R⁸ is derived from ricinoleic acid, 10-hydroxy stearic acid, 12-hydroxy stearic acid or lesquerolic acid, and
at least one R⁹ is derived from oleic acid, ricinoleic acid or stearic acid,
and m is 1, 2, 3, 4 or 5.

More preferably, X=O,

and all groups R⁸ are derived from ricinoleic acid, 10-hydroxy stearic acid, 12-hydroxy stearic acid or lesquerolic acid, and
all groups R⁹ are derived from oleic acid, ricinoleic acid or stearic acid, and m is 1, 2, 3, 4 or 5.

In still another preferred embodiment of the invention, in the organic ammonium salt according to the invention each moiety of the general formula (VI), (VI*), (VIII or (VIII*) contains at least one R⁹ selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene,

preferably each moiety of the general formula (VI), (VI*), (VIII or (VIII*) contains at least one R⁸ selected optionally hydroxyl-substituted from hexadecylene, heptadecylene, octadecylene, nonadecylene, eicosylene, hexadecenylene, heptadecenylene, octadecenylene, nonadecenylene, eicosenylene,
and m is 1, 2, 3, 4 or 5,
and more preferably at least one R⁸ in each moiety of the general formula (VI), (VI*), (VIII or (VIII*) is derived from ricinoleic acid, 10-hydroxy stearic acid, 12-hydroxy stearic acid or lesquerolic acid,
and m is 1, 2, 3, 4 or 5.

Even more preferably, every R⁸ in each moiety of the general formula (VI), (VI*), (VIII or (VIII*) is derived from ricinoleic acid, 10-hydroxy stearic acid, 12-hydroxy stearic acid or lesquerolic acid,

and m is 1, 2, 3, 4 or 5.

In a preferred embodiment of the invention, the carboxylate anion (COO⁻) group-comprising anion or anions of the organic ammonium salt according to the invention comprise the linear carboxylate anion A⁻ of the formula

⁻O—C(O)—R⁷—(O—C(O)—R⁸)ₘ₊₁—O—C(O)—R⁹

with m, R⁸ and R⁹ as defined above,
R⁷ is independently selected from a divalent optionally substituted straight-chain, cyclic or branched, saturated or unsaturated hydrocarbon radical which have up to 36 carbon atoms, and
the total number of carbon atoms in R⁸+R⁹ (Σ carbon atoms of R⁸ and R⁹ per anion) is 19 to 300, preferably 25 to 300, more preferably 35 to 300, even more preferably 50 to 300, specifically 35 to 200, more specifically 35 to 150, even more specifically 50 to 150,
and wherein R⁷ and R⁸ are preferably derived from lactic acid, ricinoleic acid, lesquerolic acid, 10-hydroxy stearic acid, 12-hydroxy stearic acid, 14-hydroxy tetradecanoic acid, most preferably from ricinoleic acid or lesquerolic acid,
R⁹ is preferably derived from octadecanoic acid, eicosanoic acid, docosanoic acid, 2-ethyl hexanoic acid, 2,2-dimethyl propionic acid, neodecanoic acid, oleic acid, and
m is preferably 1 to 10, more preferably 1 to 6, even more preferably 1 to 6, specifically 1, 2, 3, 4, 5, 6, 7.

In a further preferred embodiment of the invention, the carboxylate anion (COO⁻) group-comprising anion or anions of the organic ammonium salt according to the invention comprise the linear carboxylate anion A⁻ of the formula

⁻O—C(O)—R⁷—(O—C(O)—R⁸)ₘ₊₁—O—C(O)—R⁹

wherein m, R⁷, R⁸ and R⁹ are as defined above, and in said formula R⁷, R⁸ and R⁹ are selected as follows:

| R⁷ bearing the | | |
|---|---|---|
| carboxylate function | | R⁹ at the terminus |
| derived from | R⁸ derived from | derived from |
| unsaturated acid, in | unsaturated acid, in | unsaturated acid, in |
| particular ricinoleic acid | particular ricinoleic acid | particular oleic acid |
| or lesquerolic acids | or lesquerolic acid | |
| unsaturated acid, in | unsaturated acid, in | saturated acid, in |
| particular ricinoleic acid | particular ricinoleic acid | particular stearic acid |
| or lesquerolic acid | or lesquerolic acid | |
| unsaturated acid, in | saturated acid, in | saturated acid, in |
| particular ricinoleic acid | particular 12- | particular stearic acid |
| or lesquerolic acids | hydroxystearic acid | |
| saturated acid, in | saturated acid, in | saturated acid, in |
| particular 12- | particular 12- | particular stearic acid |
| hydroxystearic acid | hydroxystearic acid | |
| saturated acid, in | saturated acid, in | unsaturated acid, in |
| particular 12- | particular 12- | particular oleic acid |
| hydroxystearic acid | hydroxystearic acid | |
| saturated acid, i.e. 12- | unsaturated acid, in | unsaturated acid, in |
| hydroxystearic acid | particular ricinoleic acid | particular oleic acid |
| | or lesquerolic acid | |
| saturated acid, in | unsaturated acid, in | saturated acid, in |
| particular 12- | particular ricinoleic acid | particular stearic acid |
| hydroxystearic acid | or lesquerolic acid | |
| unsaturated acid, in | saturated acid, in | unsaturated acid, in |
| particular ricinoleic acid | particular 12- | particular oleic acid |
| or lesquerolic acid | hydroxystearic acid | |

It is preferred that all sequences are selected according to the above table.

It is further preferred that m is 1 to 10, more preferably 1 to 6, specifically 1, 2, 3, 4, 5, 6, or 7

In still another preferred embodiment of the invention, the carboxylate anion (COO⁻) group-comprising anion or anions of the organic ammonium salt according to the invention comprise branched linear polymeric fatty acid carboxylates, in particular branched linear polymeric fatty acid carboxylates derived from partial esters of polyfunctional carboxylic acids, in particular of the dicarboxylic acids succinic acid and maleic acid, with castor oil or lesquerella oil or hydroxy fatty acid esterified glycerol, containing at least one moiety of the formula

Glycerol-O—C(O)—R⁸—(O—C(O)—R⁸)ₘ—O—C(O)—R⁹

wherein
R⁸ is as defined above and preferably derived from lactic acid, ricinoleic acid, lesquerolic acid, 10-hydroxy stearic acid, 12-hydroxy stearic acid, 14-hydroxy tetradecanoic acid, most preferably from ricinoleic acid or lesquerolic acid,
R⁹ is as defined above and preferably derived from octadecanoic acid, eicosanoic acid, docosanoic acid, 2-ethyl hexanoic acid, 2,2-dimethyl propionic acid, neodecanoic acid, oleic acid, succinic acid and maleic acid, succinic acid anhydride and maleic acid anhydride, and in the moieties of the above-shown formula
m=0 to 20, preferred 1 to 20, more preferred 1 to 10, even more preferred 1 to 6, specifically 0, 1, 2, 3, 4, 5, 6, 7 and
the total number of carbon atoms in R⁸+R⁹ (Σ carbon atoms of Wand R⁹) being 19 to 300, preferably 25 to 300, more preferably 35 to 300, even more preferably 50 to 300, specifically 35 to 200, more specifically 35 to 150, even more specifically 50 to 150.

According to the invention, the term “branched linear polymeric fatty acid carboxylate” refers to a branched compound comprising a branched structure, two or more linear polymeric fatty acid moieties of the formula

—O—C(O)—R⁸—(O—C(O)—R⁸)ₘ—O—C(O)—R⁹

and at least one carboxylate group.

The term “polyfunctional carboxylic acids” refers to carboxylic acid compounds comprising at least one further carboxylic acid group.

The term

“esterified glycerol containing at least one moiety of the formula

Glycerol-O—C(O)—R⁸—(O—C(O)—R⁸)ₘ—O—C(O)—R⁹”

refers to a glycerol molecule in which at least one of the glycerol's three hydroxy groups is bonded to a moiety of the formula

—R⁸—(O—C(O)—R⁸)ₘ—O—C(O)—R⁹

with R⁸, R⁹ and m as defined according to this embodiment group via an ester group.

An example of an anion according to the embodiment is the anion obtained by deprotonation of the structure

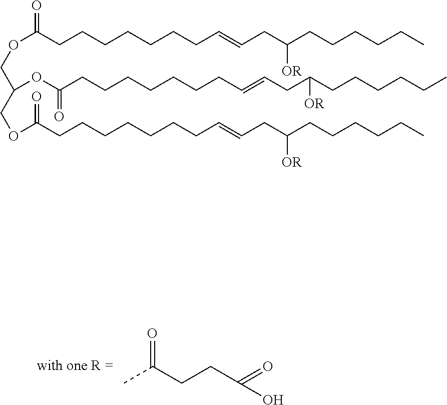

and the remaining two R groups=

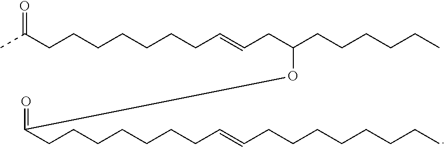

In a further preferred embodiment of the invention, the carboxylate anion (COO⁻) group-comprising anion or anions of the organic ammonium salt according to the invention comprise branched linear polymeric fatty acid carboxylates containing at least one moiety of the formula

Glycerol-O—C(O)—R⁸—(O—C(O)—R⁸)ₘ—O—C(O)—R⁹

wherein at least one sequence of the general formula

Glycerol-O—C(O)—R⁸—(O—C(O)—R⁸)ₘ—O—C(O)—R⁹

with m, R⁸ and R⁹ as defined above
is selected from

| R⁸ adjacent to glycerol | | R⁹ at the terminus | |
|---|---|---|---|
| derived from | other R⁸ derived from | derived from | |
| unsaturated acid, in | unsaturated acid, in | unsaturated acid, in | |
| particular ricinoleic | particular ricinoleic | particular oleic acid | |
| acid or lesquerolic acid | acid or lesquerolic acid | | |
| unsaturated acid, in | unsaturated acid, in | saturated acid, in | |
| particular ricinoleic | particular ricinoleic | particular stearic acid | |
| acid or lesquerolic acid | acid or lesquerolic acid | | |
| unsaturated acid, in | saturated acid, in | saturated acid, in | |
| particular ricinoleic | particular 12- | particular stearic acid | |
| acid or lesquerolic acid | hydroxystearic acid | saturated acid, in | |
| saturated acid, in | saturated acid, in | particular stearic acid | |
| particular 12- | particular 12- | | |
| hydroxystearic acid | hydroxystearic acid | | |
| saturated acid, in | saturated acid, in | unsaturated acid, in | |
| particular 12- | particular 12- | particular oleic acid | |
| hydroxystearic acid | hydroxystearic acid | | |
| saturated acid, i.e. 12- | unsaturated acid, in | unsaturated acid, in | |
| hydroxystearic acid | particular ricinoleic | particular oleic acid | |
| | acid or lesquerolic acid | | |
| saturated acid, in | unsaturated acid, in | saturated acid, in | |
| particular 12- | particular ricinoleic | particular stearic acid | |
| hydroxystearic acid | acid or lesquerolic acid | | |
| unsaturated acid, in | saturated acid, in | unsaturated acid, in | |
| particular ricinoleic | particular 12- | particular oleic acid | |
| acid or lesquerolic acid | hydroxystearic acid | | , |

preferably two sequences are selected according to the above table, while the third hydroxy group of the glycerol group is functionalized to bear a carboxylate group-containing residue.

It is further preferred that m is 0 to 10, more preferably 1 to 6, specifically 0, 1, 2, 3, 4, 5, 6, or 7.

In another preferred embodiment of the invention, the carboxylate anion (COO⁻) group-comprising anion or anions of the organic ammonium salt according to the invention comprise branched or dendritic polymeric fatty acid carboxylate anions A⁻ of the formula (V), wherein in the formula (VI) or (VI*)

R⁷, X, R⁸, R⁹, R⁹* and m are as described above,
wherein the R⁷ group bears at least one anionic carboxylate group, and is preferably derived from bis-hydroxy mono carboxylic acids, such as glycerolic acid and 2,2-bis-(hydroxymethyl) propionic acid,
R⁸ is as defined above and is preferably derived from lactic acid, ricinoleic acid, lesquerolic acid, 10-hydroxy stearic acid, 12-hydroxy stearic acid, 14-hydroxy tetradecanoic acid, most preferably from ricinoleic acid or lesquerolic acid,
R⁹ is as defined above and preferably derived from octadecanoic acid, eicosanoic acid, docosanoic acid, 2-ethyl hexanoic acid, 2,2-dimethyl propionic acid, neodecanoic acid, and stearic acid, oleic acid,
and T in R⁹* is preferably derived from octadecanoic acid, eicosanoic acid, docosanoic acid, 2-ethyl hexanoic acid, 2,2-dimethyl propionic acid, neodecanoic acid, stearic acid and oleic acid
m=1 to 20, preferred 1 to 10, more preferred 1 to 6, even more preferred 1 to 6, specifically 1, 2, 3, 4, 5, 6, 7 and
the total number of carbon atoms in R⁸+R⁷ (Σ carbon atoms of R⁸ and R⁹) being 19 to 300, preferably 25 to 300, more preferably 35 to 300, even more preferably 50 to 300, specifically 35 to 200, more specifically 35 to 150, even more specifically 50 to 150.

In a further preferred embodiment, the carboxylate anion (COO⁻) group-comprising anion or anions of the organic ammonium salt according to the previous embodiment comprise an anion or anions derived from branched or dendritic poly fatty acid structures

as described above,
wherein at least one sequence of the general formula
R⁷(—X—C(O)—R⁸)ₘ—X—C(O)—R⁹ with X and m as defined above is selected from

| R⁸ adjacent to R⁷ | | | |
|---|---|---|---|
| bearing a carboxylate | | | |
| function (for example | | | |
| 2,2-bis- | | | |
| (hydroxymethyl) | | R⁹ at the terminus | |
| propionic acid) derived | | (carboxylate free) | |
| from | other R⁸ derived from | derived from | |
| unsaturated acid, in | unsaturated acid, in | unsaturated acid, in | |
| particular ricinoleic | particular ricinoleic | particular oleic acid | |
| acid or lesquerolic acid | acid or lesquerolic acid | | |
| unsaturated acid, in | unsaturated acid, in | saturated acid, in | |
| particular ricinoleic | particular ricinoleic | particular stearic acid | |
| acid or lesquerolic acid | acid or lesquerolic acid | | |
| unsaturated acid, in | saturated acid, in | saturated acid, in | |
| particular ricinoleic | particular 12- | particular stearic acid | |
| acid or lesquerolic acid | hydroxystearic acid | | |
| saturated acid, in | saturated acid, in | saturated acid, in | |
| particular 12- | particular 12- | particular stearic acid | |
| hydroxystearic acid | hydroxystearic acid | | |
| saturated acid, in | saturated acid, in | unsaturated acid, in | |
| particular 12- | particular 12- | particular oleic acid | |
| hydroxystearic acid | hydroxystearic acid | | |
| saturated acid, i.e. 12- | unsaturated acid, in | unsaturated acid, in | |
| hydroxystearic acid | particular ricinoleic | particular oleic acid | |
| | acid or lesquerolic acid | | |
| saturated acid, in | unsaturated acid, in | saturated acid, in | |
| particular 12- | particular ricinoleic | particular stearic acid | |
| hydroxystearic acid | acid or lesquerolic acid | | |
| unsaturated acid, in | saturated acid, in | unsaturated acid, in | |
| particular ricinoleic | particular 12- | particular oleic acid | |
| acid or lesquerolic acid | hydroxystearic acid | | , |

preferably the groups of all such sequences are selected according to the above table.

In a further preferred embodiment of the invention, the carboxylate anion (COO⁻) group-comprising anion or anions of the organic ammonium salt according to the invention comprise carboxylate anions A⁻ of the type

R⁷[(—C(O)—X—R⁸)ₘ₊₁—C(O)—X—R⁹C(O)O⁻]_{q}, or (⁻OC(O))_{q-1}—R⁷—(C(O)—X—R⁸)ₘ₊₁—C(O)—X—R⁹C(O)O⁻

wherein X=O, R⁷, R⁸, R⁹, m and q are as defined above and
preferably q=2 to 4, specifically 2, 3, 4,
R⁸ is as defined above and preferably derived from lactic acid, ricinoleic acid, lesquerolic acid, 10-hydroxy stearic acid, 12-hydroxy stearic acid, 14-hydroxy tetradecanoic acid, most preferably from ricinoleic acid or lesquerolic acid,
R⁷ is as defined above and preferably derived from maleic acid, succinic acid, trimellitic acid, pyromellitic acid,
and
m=0 to 20, preferably 1 to 20, more preferably 1 to 10, even more preferably 1 to 6, even further preferably 1 to 6, specifically 1, 2, 3, 4, 5, 6, 7, and
the total number of carbon atoms in R⁸+R⁹ (Σ carbon atoms of R⁸ and R⁹ per anion) is 19 to 300, preferably 25 to 300, more preferably 35 to 300, even more preferably 50 to 300, specifically 35 to 200, more specifically 35 to 150, even more specifically 50 to 150.

In an even further preferred embodiment of the invention, the carboxylate anion (COO⁻) group-comprising anion or anions of the organic ammonium salt according to the invention comprise carboxylate anions A⁻ of the type

R⁷[(—C(O)—X—R⁸)ₘ₊₁—C(O)—X—R⁹C(O)O⁻]_{q}, or (⁻OC(O))_{q-1}—R⁷—(C(O)—X—R⁸)ₘ₊₁—C(O)—X—R⁹C(O)O⁻

with X=O, R⁷, R⁸, R⁹, m and q are as defined above, and wherein at least one polyacid ester sequence in the general formulas

R⁷[(—C(O)—X—R⁸)ₘ₊₁—C(O)—X—R⁹C(O)O⁻]_{q}, and (⁻OC(O))_{q-1}—R⁷—(C(O)—X—R⁸)ₘ₊₁—C(O)—X—R⁹C(O)O⁻

is selected from

| R⁹ at the carboxylate | | R⁸ adjacent to R⁷ | |
|---|---|---|---|
| terminus derived from | R⁸ derived from | derived from | |
| unsaturated acid, in | unsaturated acid, in | unsaturated acid, in | |
| particular ricinoleic | particular ricinoleic | particular ricinoleic | |
| acid or lesquerolic acid | acid or lesquerolic acid | acid or lesquerolic acid | |
| unsaturated acid, in | unsaturated acid, in | saturated acid, in | |
| particular ricinoleic | particular ricinoleic | particular 12- | |
| acid or lesquerolic acid | acid or lesquerolic acid | hydroxystearic acid | |
| unsaturated acid, in | saturated acid, in | saturated acid, in | |
| particular ricinoleic | particular 12- | particular 12- | |
| acid or lesquerolic acid | hydroxystearic acid | hydroxystearic acid | |
| saturated acid, in | saturated acid, in | saturated acid, in | |
| particular 12- | particular 12- | particular 12- | |
| hydroxystearic acid | hydroxystearic acid | hydroxystearic acid | |
| saturated acid, in | saturated acid, in | unsaturated acid, in | |
| particular 12- | particular 12- | particular ricinoleic | |
| hydroxystearic acid | hydroxystearic acid | acid or lesquerolic acid | |
| saturated acid, i.e. 12- | unsaturated acid, in | unsaturated acid, in | |
| hydroxystearic acid | particular ricinoleic | particular ricinoleic | |
| | acid or lesquerolic acid | acid or lesquerolic acid | |
| saturated acid, in | unsaturated acid, in | saturated acid, in | |
| particular 12- | particular ricinoleic | particular 12- | |
| hydroxystearic acid | acid or lesquerolic acid | hydroxy stearic acid | |
| unsaturated acid, in | saturated acid, in | unsaturated acid, in | |
| particular ricinoleic | particular 12- | particular ricinoleic | |
| acid or lesquerolic acid | hydroxystearic acid | acid or lesquerolic acid | , |

preferably all such sequences in an anion are selected according to the above table,

Preferably, m is 1 to 10, specifically 1, 2, 3, 4, 5, 6, 7, or 8, and even more preferably in addition q is 1-6, most preferably 2 to 4.

In another preferred embodiment of the invention, the organic ammonium-group-comprising cation is selected from cations according to the general formula

R¹(—F)ₓ (I) as defined above, wherein

x is 1 to 10, preferred 1 to 5, more preferred 1, 2, 3, 4, 5, most preferred 1 and 2,
F can be the same or different and is represented by the general formula (II):

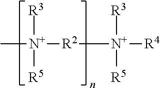

wherein R¹, R², R³, R⁴, R⁵ and n are as defined above, and preferably
n=0-100, even more preferred 0-50, even further preferred 0-20, most preferred 0, 1, 2, 3, 4 or 5.

In still another preferred embodiment of the invention, the organic ammonium-group-comprising cation is selected from cations according to the general formula

R¹(—F)ₓ (I)

as defined above, wherein x=2 and the cation is represented by the general formula (V):

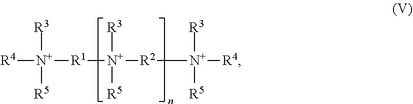

wherein R¹, R², R³, R⁴, R⁵ and n are as defined above, and preferably
n=0-1000, preferred 0-100, more preferred 0-50, even more preferred 0-20, most preferred 0, 1, 2, 3, 4 or 5.

An example of the cations according to this embodiment are cations derived from the amino compound

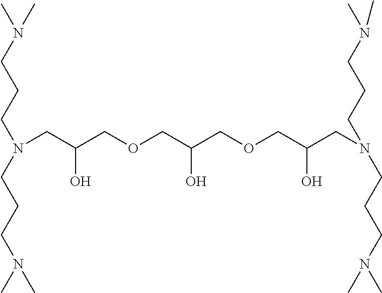 [SMILES: CN(C)CCCN(CCCN(C)C)CC(O)COCC(O)COCC(O)CN(CCCN(C)C)CCCN(C)C]

In a further preferred embodiment of the invention, the organic ammonium-group-comprising cation is selected from cations according to the general formula

R¹(—F)ₓ (I) as defined above, wherein,

x is 1 to 10, preferred 1 to 5, more preferred 1, 2, 3, 4, or 5, most preferred 1 or 2,
R¹ is selected from monovalent to decavalent, optionally substituted hydrocarbon radicals which have up to 1000 carbon atoms, preferred 2 to 300 carbon atoms, more preferred 3 to 200 carbon atoms, even more preferred 3 to 150 carbon atoms, specifically 3 to 50 carbon atoms, more specifically 3 to 20 carbon atoms may contain optionally one or more groups selected from —O—, —NH—, —C(O)—, —C(S)—, tertiary amino groups

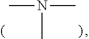

groups and can be substituted by —OH groups and halide groups,
preferably R¹ is a C3-C18 glycerol-based polyether radical or a C1-C8 linear alkylene radical, and
F has the general formula (XVII), which corresponds to formula (II) with n being equal to 0:

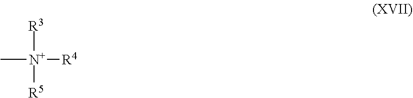

and the groups F bind to a carbon atom of R¹,
wherein
R³, R⁴, R⁵ are independently selected from hydrogen and optionally substituted straight-chain, cyclic or branched, saturated, unsaturated or aromatic hydrocarbon radicals which have up to 300 carbon atoms, preferred 1 to 200 carbon atoms, more preferred 1 to 150 carbon atoms, even more preferred 1 to 50 carbon atoms, specifically 1 to 20 carbon atoms, more specifically 1 to 10 carbon atoms which optionally contain one or more croups selected from —O—, —NH—, —C(O)—, —C(S)—, tertiary amino groups

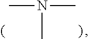

and can be substituted by OH, preferably R³ to R⁵ are C1-C8 linear alkyl groups, such as methyl ethyl, propyl or butyl.

In a preferred embodiment according to the invention, the carboxylate anion (COO⁻) group-comprising anion or anions are preferably mono- to pentacontavalent, more preferably mono- to decavalent, even more preferably mono- to pentavalent, most preferably pentavalent, tetravalent, trivalent, divalent or monovalent anions.

In another preferred embodiment of the invention, the carboxylate anion (COO⁻) group-comprising anion or anions comprise at least one moiety of the general formulas (XI) or (XII):

—X—C(O)—R^{x}—(X—C(O)—R^{x})ₘ—X—C(O)—R⁹ (XI) or

—X—C(O)—R^{x}—(X—C(O)—R^{x})ₘ—X—C(O)—R⁹ (XII)

wherein

### X is O or NR¹¹,

m=1 to 20, preferred 1 to 10, more preferred 1 to 6, even more preferred 2 to 6, specifically 1, 2, 3, 4, 5, 6 and
the total number of carbon atoms in R^{x}+R⁹ (Σ carbon atoms R^{x}, R⁹ per anion) is 19 to 300, preferred 25 to 300, more preferred 35 to 300, even more preferred 50 to 300, specifically 35 to 200, more specifically 35 to 150, even more specifically 50 to 150,
R¹¹ is as defined above and preferably selected from the group consisting of hydrogen, n-, iso-, or tert.-C1-C22-alkyl, more preferred hydrogen,
R^{x} is an optionally OH, —O—C(O)—R⁹, —O—C(O)—R⁸—(O—C(O)—R⁸)₀₋₁₉—O—C(O)—R⁹ substituted straight-chain, cyclic or branched, saturated or unsaturated hydrocarbon radical which has 1 to 36 carbon atoms excluding the carbon atoms of R⁸ and R⁹ group-containing substituents, preferred 1 to 24 carbon atoms, more preferred 1 to 18 carbon atoms, even more preferred 8 to 18 carbon atoms, and which is preferably derived from monohydroxy carboxylic acids, in particular glycolic acid, lactic acid, 2-hydroxy butyric acid, 3-hydroxy-butyric acid, 4-hydroxy butyric acid, 14-hydroxy tetradecanoic acid, 10-hydroxy stearic acid, 12-hydroxy stearic acid, lesquerolic acid, ricinoleic acid, or dihydroxy carboxylic acids, in particular 2,2′-di-hydroxymethyl propanoic acid, 9,10-dihydroxy stearic acid, or polyhydroxy carboxylic acids, in particular gluconic acid,
R⁸ is as defined above,
R⁹ is selected from optionally substituted straight-chain, cyclic or branched, saturated or unsaturated hydrocarbon radicals which have 1 to 36 carbon atoms, preferred 1 to 24 carbon atoms, more preferred 1 to 18 carbon atoms, even more preferred 8 to 18 carbon atoms, preferably derived from acetic acid, octanoic acid, nonanoic acid, decanoic acid, dodecanoic acid, tetradecanoic acid, hexadecanoic acid, octadecanoic acid, eicosanoic acid, docosanoic acid, 2-ethyl hexanoic acid, 2,2-dimethyl propionic acid, 2,2-dimethyl heptanoic acid, 2,2-dimethyl octanoic acid, neodecanoic acid, undecyl-10-en-ic acid, oleic acid, linoleic acid, linolenic acid, erucic acid, and
it is preferred when R⁹ is independently selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, more preferably independently selected from optionally hydroxyl-substituted hexadecylene, heptadecylene, octadecylene, nonadecylene, eicosylene, hexadecenylene, heptadecenylene, octadecenylene, nonadecenylene, eicosenylene, most preferably each R⁹ is independently derived from ricinoleic acid, 10-hydroxy stearic acid, 12-hydroxy stearic acid or lesquerolic acid.

In a preferred embodiment according to the invention, the organic ammonium-group-comprising cation is selected from cations according to the general formula

R¹(—F)ₓ (I)

as defined above, wherein R¹ is selected from the group consisting of:
monovalent to octadecavalent, preferably divalent to octadecavalent, more preferably divalent to hexavalent, even more preferably divalent, trivalent and tetravalent optionally —O—, —C(O)—, OH or amido substituted straight-chain, cyclic or branched, saturated, unsaturated or aromatic hydrocarbyl groups, derived from primary, secondary and tertiary amines as well as quaternary ammonium compounds having at least one, preferred more than one, more preferred three, even more preferred more than three carbon atoms,
   in particular derived from
primary amines,
   such as C1 to C24 primary amines, i.e. methylamine, ethylamine, propylamine i-propylamine, butylamine, hexylamine, cyclohexylamine, octylamine, 2-ethylhexylamine, decylamine, undecenylamine, dodecylamine, tetradecylamine, hexadecylamine, octadecylamine, oleylamine,
   OH functionalized primary amines, i.e. ethanolamine, glucamine, aminoglycerol, polyether based primary amines, i.e. polyethylene oxide and polypropylene oxide based mono- di- and trifunctional primary amines of the Jeffamine® series, e.g. Jeffamine® M 600, 1000, 2005, 2070, Jeffamine® XTJ-435, 436, Jeffamine® D 230, 400, 2000, 4000 Jeffamine® ED HK-511, 600, 900, 2003, Jeffamine® EDR 148, 176, Jeffamine® T 403, 3000, 5000,
   derived from
condensation products of epoxy compounds, in particular glycidyl ethers, with alcohols, in particular methanol, ethanol, 2-propanol, 1-butanol, t-butanol, undec-10-en-ol, oleyl alcohol, stearyl alcohol, 1,2-propanediol, 1,3-propanediol, 1,3-butanediol, 1,4-butanediol, 1,2 hexanediol, 1,6-hexanediol, glycerol, diglycerol, triglycerol and higher linear or branched oligoglycerols, trimethylol propane, castor oil (ricinoleic acid triglyceride), pentaerythritol, sorbitol, poly(alkylene oxides), such as (ethylene oxide)-, (propylene oxide)- and/or (butylene oxide)-based polyethers, e.g. derived from polyethylene glycols, like diethylene glycol, triethylene glycol, tetraethylene glycol, and pentaethylene glycol etc., or derived from polypropylene glycols, like dipropylene glycol (e.g., derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed (ethylene oxide) and (butylene oxide)-based copolyethers, derived from mixed (propylene oxide)- and (butylene oxide)-based copolyethers, and derived from mixed (ethylene oxide)- and (propylene oxide)- and (butylene oxide)-based copolyethers, or preferred glycidyl esters, with acids, in particular neodecanoic acid, with ammonia,
derived from basic amino acids, such as lysine, arginine, histidine or their ester or amido derivatives,
derived from secondary amines such as C1 to C24 secondary amines, in particular from N-methylamines such asdimethylamine, N-mehyloctylamine, N-methyldodecylamine, N-methyloctadecylamine, N-ethylamines such as diethylamine, N-butylamines such as dibutylamine, cyclic amines such as piperazine, morpholine,
   OH functionalized secondary amines, such as diethanolamine, N-methylglucamine, N-methylaminoglycerol,
   polyether based secondary amines such as polyethylene oxide and polypropylene oxide based di-functional secondary amines of the Jeffamine® series, in particular i.e. Jeffamine® SD 231, 401, 404, 2001 (Huntsman), or polyethyleneimines, i.e. the Lupasol® types (BASF),
derived from condensation products of epoxy compounds, in particular glycidyl ethers, with alcohols, in particular methanol, ethanol, 2-propanol, 1-butanol, t-butanol, undec-10-en-ol, oleyl alcohol, stearyl alcohol, 1,2-propanediol, 1,3-propanediol, 1,3-butanediol, 1,4-butanediol, 1,2 hexanediol, 1,6-hexanediol, glycerol, diglycerol, triglycerol and higher linear or branched oligoglycerols, trimethylol propane, castor oil (ricinoleic acid triglyceride), pentaerythritol, sorbitol, poly(alkylene oxides), such as (ethylene oxide)-, (propylene oxide)- and/or (butylene oxide)-based polyethers, e.g. derived from polyethylene glycols, like diethylene glycol, triethylene glycol, tetraethylene glycol, and pentaethylene glycol etc., or derived from polypropylene glycols, like dipropylene glycol (e.g., derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed (ethylene oxide) and (butylene oxide)-based copolyethers, derived from mixed (propylene oxide)- and (butylene oxide)-based copolyethers, and derived from mixed (ethylene oxide)- and (propylene oxide)- and (butylene oxide)-based copolyethers, or preferred glycidyl esters, with acids, in particular neodecanoic acid, with primary amines, i.e. methylamine, ethylamine, butylamine, cyclohexylamine, octylamine, 2-ethylhexylamine, undecenylamine, dodecylamine, hexadecylamine, stearylamine, oleylamine, ethylenediamine, 1,2-propylenediamine, 1,3-propylenediamine, 1,4-butanediamine, 1,6-hexanediamine, 1,10-decanediamine, 1,12-dodecanediamine,
   basic amino acids, such as N-methylysine,
derived from tertiary amines, in particular
   trimethylamine, triethylamine, tributylamine, N,N-dimethylethanolamine, N,N-dimethylpropanolamine, N-methyl imidazole, N,N,N′,N′-tetramethyl-1,2-diaminoethane, N,N, N′,N′-tetramethyl-1,4-diaminobutane, N,N,N′,N′-tetramethyl-1,6-diaminohexane, N,N,N′,N″,N″-pentamethyl-diethylenetriamine, N,N,N′,N″,N″-pentamethyl-dipropylenetriamine, bis-(2-dimethylaminoethyl)ether, bis-(2-dimethylaminopropyl)ether, 2,2′-dimorpholinodiethylether, N,N-bis-(3-dimethylaminopropyl)-N-isopropanolamine, trimethylaminoethyl-ethanolamine, 1,3,5-tris(3-(dimethylamino)propyl)-hexahydro-s-triazine, N-methylmorpholine, N-ethylmorpholine, N,N′-dimethylpiperazine, N,N-dimethylbenzylamine, N,N-dimethylcyclohexylamine,
derived from condensation products of epoxy compounds, in particular glycidyl ethers, with alcohols, in particular methanol, ethanol, 2-propanol, 1-butanol, t-butanol, undec-10-en-ol, oleyl alcohol, stearyl alcohol, 1,2-propanediol, 1,3-propanediol, 1,3-butanediol, 1,4-butanediol, 1,2 hexanediol, 1,6-hexanediol, glycerol, diglycerol, triglycerol and higher linear or branched oligoglycerols, trimethylol propane, castor oil (ricinoleic acid triglyceride), pentaerythritol, sorbitol, poly(alkylene oxides), such as (ethylene oxide)-, (propylene oxide)- and/or (butylene oxide)-based polyethers, e.g. derived from polyethylene glycols, like diethylene glycol, triethylene glycol, tetraethylene glycol, and pentaethylene glycol etc., or derived from polypropylene glycols, like dipropylene glycol (e.g., derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed (ethylene oxide) and (butylene oxide)-based copolyethers, derived from mixed (propylene oxide)- and (butylene oxide)-based copolyethers, and derived from mixed (ethylene oxide)- and (propylene oxide)- and (butylene oxide)-based copolyethers, or preferred glycidyl esters, with acids, in particular neodecanoic acid, with primary or secondary amino functionalized amines, in particular methylamine, ethylamine, butylamine, cyclohexylamine, octylamine, 2-ethylhexylamine, undecenylamine, dodecylamine, hexadecylamine, stearylamine, oleylamine, ethylenediamine, 1,2-propylenediamine, 1,3-propylenediamine, 1,4-butanediamine, 1,6-hexanediamine, 1,10-decanediamine, 1,12-dodecanediamine, N,N-dimethylethylenediamine, N,N-dimethylpropylenediamine, N,N,N′,N′-tetramethyl-diethylenetriamine, N,N,N′,N′-tetramethyl-dipropylenetriamine, N-methylpiperazine;
derived from condensation products of carboxylic acids, in particular C6-C24 fatty acids, i.e. hexanoic acid, 2-ethylhexanoic acid, octanoic acid, decanoic acid, undecenoic acid, dodecanoic acid, tetradecanoic acid, hexadecenoic acid, octadecanoic acid, oleic acid, ricinoleic acid, lesquerolic acid, 12-hydroxy stearic acid, di- and higher carboxylic acids, i.e. succinic acid, sebacic acid, trimellitic acid with primary-tertiary amines, i.e. N,N-dimethylpropylenediamine, N,N-dimethylethylenediamine, N-dodecyl-N-bis-(propylamine), with secondary-tertiary amines N,N,N′,N′-tetramethyl-diethylenetriamine, tetramethyl-dipropylenetriamine, N-methylpiperazine; N-ethylpiperazine,
derived from mono quaternary ammonium compounds, such as
   C1-C30 tetra alkyl substituted ammonium compounds having preferentially Cl⁻, Br, CH₃—O—SO₃⁻ or OH− counter ions,
   i.e. compounds having identical alkyl substituents, i.e. (C₂H₅)₄N⁺ Cl⁻, (C₂H₅)₄N⁺ OH⁻, (C₄H₉)₄N⁺ Br⁻, (C₄H₉)₄N⁺ OH⁻,
   compounds having different alkyl substituents, preferably being a mono-long alkyl-tri short alkyl quaternized ammonium salt or a di-long alkyl-di short alkyl quaternized ammonium salt wherein one or two alkyl substituents are selected from an aliphatic group of from about 8 to about 30 carbon atoms or an aromatic, alkoxy, polyoxyalkylene, alkylamido, hydroxyalkyl, aryl or alkylaryl group having up to about 30 carbon atoms; the other alkyl groups are independently selected from an aliphatic group of from about 1 to about 8 carbon atoms or an aromatic, alkoxy, polyoxyalkylene, alkylamido, hydroxyalkyl, aryl or alkylaryl group having up to about 8 carbon atoms, the aliphatic groups can contain, in addition to carbon and hydrogen atoms, ether linkages, and other groups such as amino groups, the longer chain aliphatic groups, e.g., those of about 8 carbons, or higher, can be saturated or unsaturated,
   preferably, one alkyl group is selected from an alkyl group of from about 8 to about 30 carbon atoms, more preferably from about 14 to about 26 carbon atoms, still more preferably from about 14 to 22 carbon atoms; the other alkyl groups are independently selected from the group consisting of CH₃, C₂H₅, C₂H₄OH, CH₂C₆H₅, and mixtures thereof; and the counter ion is selected from the group consisting of Cl−, Br−, CH₃OSO₃⁻, and mixtures thereof,
   C1-C30 tetra alkyl substituted ammonium compounds containing ester moieties and preferably having Cl⁻, Br⁻, CH₃—O—SO₃⁻ counter ions, preferably being cationic saturated or unsaturated fatty acid based mono-ester and di-ester quats having 10 to 22, preferred 10 to 18 carbon atoms in the fatty acid derived alkyl chains,
   ethoxylated C10-C18 saturated and unsaturated mono-ester quats, such as cocoyl pentaethoxy methyl ammonium metho-sulfate,
   triethanolamine derived C10-C18, preferably C16-C18 saturated and unsaturated di-ester quats, such as

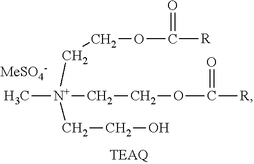

with R independently selected from C9-C17 alkyl groups or alkenyl groups, preferably R is independently selected from C15-C17 alkyl groups or alkenyl groups,
triethanolamine derived C10-C18, preferably C16-C18 saturated and unsaturated tri-ester quats, such as

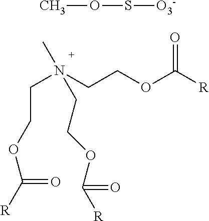

with R independently selected from C9-C17 alkyl groups or alkenyl groups, preferably R is independently selected from C15-C17 alkyl groups or alkenyl groups,
N-methyl-diethanolamine derived C10-C18, preferably C16-C18 saturated and unsaturated ester quats, such as

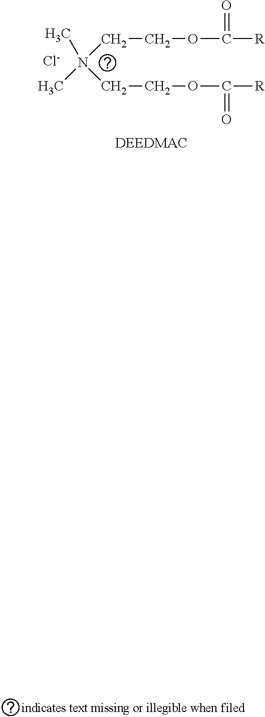

with R independently selected from C9-C17 alkyl groups or alkenyl groups, preferably R is independently selected from C15-C17 alkyl groups or alkenyl groups,
N,N-dimethyl-3-aminopropane-1,2-diol derived C10-C18, preferably C16-C18 saturated and unsaturated ester quats, such as

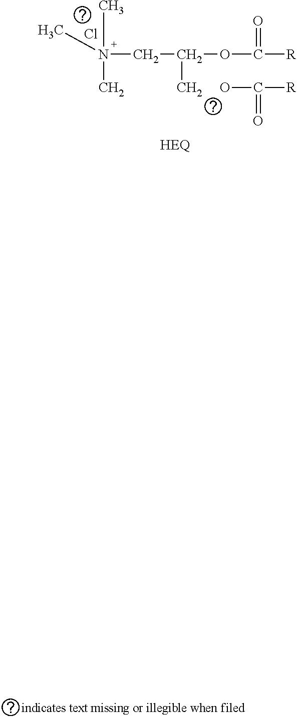

with R independently selected from C9-C17 alkyl groups or alkenyl groups, preferably R is independently selected from C15-C17 alkyl groups or alkenyl groups,
N-dimethyl-diisopropanolamine derived C16-C18 saturated and unsaturated ester quats of the formula

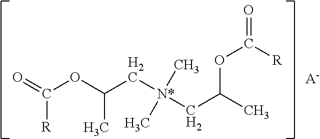

with R=C15-C17 alkyl or alkenyl group, and A⁻=monovalent cation,
in particular dioleoylisopropyl dimethylammonium methosulfate, dioleoylisopropyl dimethylammonium chloride, dipalmitoylisopropyl dimethylammonium methosulfate, dipalmitoylisopropyl dimethylammonium chloride, bis-(isostearoyl/oleoyl isopropyl) dimethylammonium methosulfate, bis-(isostearoyl/oleoyl isopropyl) dimethylammonium chloride,
derived from symmetric or asymmetric, head group bridged or tail bridged, di-quaternary ammonium compounds, gemini quats, in particular di-quaternary compounds, containing at least one, preferred at least two C10-C30, more preferred C10-C22, even more preferred C10-C18 alkyl groups,
   in particular head group bridged di-quaternary compounds,
   such as alkyl bridge-containing-, preferred C2-C30 alkyl bridge-containing di-quaternary compounds, for example

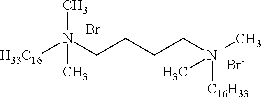

such as ether and ester bridged, preferably C4-C30 ether and ester bridges containing di-quaternary compounds, for example

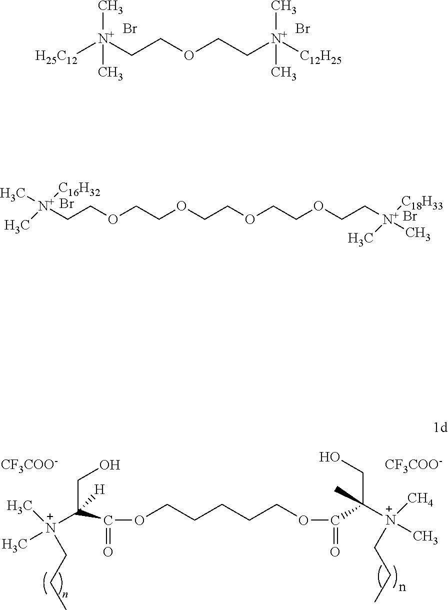

with n independently selected from 0 to 24,

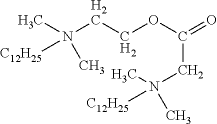

such as hydroxy alkyl-bridged, preferably glycerol, more preferably C3-C30 glycerol bridges containing di-quaternary compounds, for instance

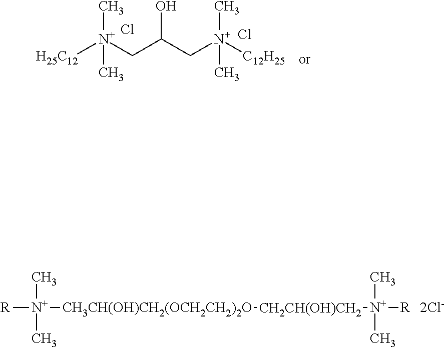

with R independently selected from C1-C25 alkyl groups or C2-C25 alkenyl groups,
such as fatty acid or fatty alcohol ester moieties, preferably C6-C30, preferably C10-C30, more preferably C10-C22, even more preferably C16-C18 fatty acid or fatty alcohol ester moieties containing di-quaternary compounds, for example

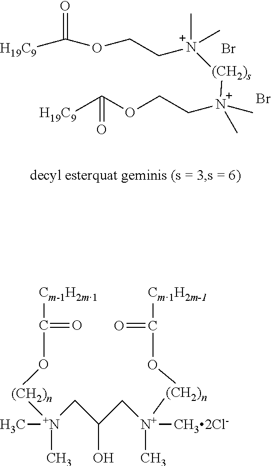

with n independently selected from 2-22, and m independently selected from 2-6,

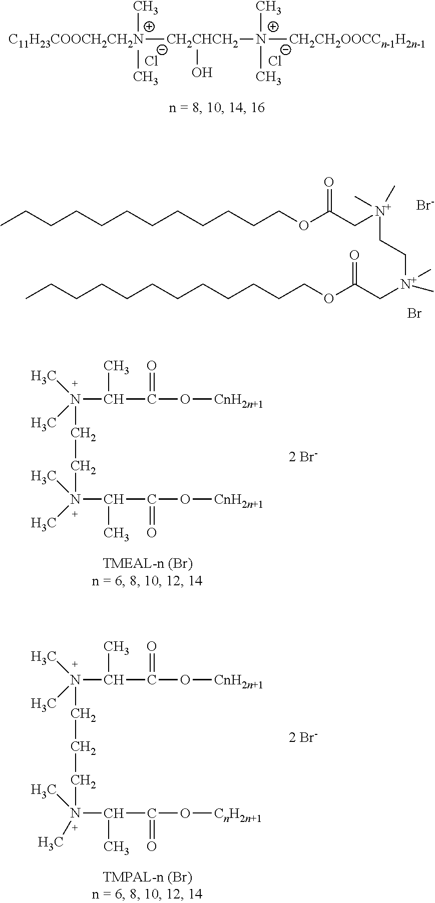

in particular derived from tri-quaternary ammonium compounds,
   preferably tri-quaternary compounds containing at least one, preferably at least two C10-C30, more preferably three C10-C22, even more preferably C10-C18 alkyl groups, for example

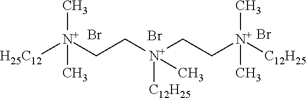

in particular derived from poly quaternary compounds, preferably INCI registered polyquaternium compounds, i.e. polyquaternium 1 to polyquaternium 113, such as the polyquaternium compounds listed in the following table

| Polyquaternium | Chemical Identity |
|---|---|
| Polyquaternium1 | Ethanol, 2,2′,2″-nitrilotris-, polymer with 1,4-dichloro-2-butene and |
| | N,N,N′,N′-tetramethyl-2-butene-1,4-diamine |
| Polyquaternium2 | Poly[bis(2-chloroethyl) ether-alt-1,3-bis[3-(dimethylamino)propyl]urea] |
| Polyquaternium4 | Hydroxyethyl cellulose dimethyl diallylammonium chloride copolymer; |
| | Diallyldimethylammonium chloride-hydroxyethyl |
| | cellulose copolymer |
| Polyquaternium5 | Copolymer of acrylamide and quaternized dimethylammoniumethyl |
| | methacrylate |
| Polyquaternium6 | Poly(diallyldimethylammonium chloride) |
| Polyquaternium7 | Copolymer of acrylamide and diallyldimethylammonium chloride |
| Polyquaternium8 | Copolymer of methyl and stearyl dimethylaminoethyl ester of |
| | methacrylic acid, quaternized with dimethylsulphate^{[2]} |
| Polyquaternium9 | Homopolymer of N,N-(dimethylamino)ethyl ester of methacrylic acid, |
| | quaternized with bromomethane |
| Polyquaternium10 | Quaternized hydroxyethyl cellulose |
| Polyquaternium11 | Copolymer of vinylpyrrolidone and quaternized dimethylaminoethyl |
| | methacrylate |
| Polyquaternium12 | Ethyl methacrylate/abietyl methacrylate/diethylaminoethyl |
| | methacrylate copolymer quaternized with dimethyl sulfate |
| Polyquaternium13 | Ethyl methacrylate/oleyl methacrylate/diethylaminoethyl methacrylate |
| | copolymer quaternized with dimethyl sulfate |
| Polyquaternium14 | Trimethylaminoethylmethacrylate homopolymer |
| Polyquaternium15 | Acrylamide-dimethylaminoethyl methacrylate methyl chloride |
| | copoU met |
| Polyquaternium16 | Copolymer of vinylpyrrolidone and quaternized vinylimidazole |
| Polyquaternium17 | Adipic acid, dimethylaminopropylamine and dichloroethylether |
| | copolymer |
| Polyquaternium18 | Azelaic acid, dimethylaminopropylamine and dichloroethylether |
| | copolymer |
| Polyquaternium19 | Copolymer of polyvinyl alcohol and 2,3-epoxypropylamine |
| Polyquaternium20 | Copolymer of polyvinyl octadecyl ether and 2,3-epoxypropylamine |
| Polyquaternium22 | Copolymer of acrylic acid and diallyldimethylammonium Chloride |
| Polyquaternium24 | Quaternary ammonium salt of hydroxyethyl cellulose reacted with a |
| | lauryl dimethyl ammonium substituted epoxide. |
| Polyquaternium27 | Block copolymer of Polyquaternium-2 and Polyquaternium-17 |
| Polyquaternium28 | Copolymer of vinylpyrrolidone and methacrylamidopropyl |
| | trimethylammonium |
| Polyquatemium29 | Chitosan modified with propylen oxide and quaternized with |
| | epichlorhydrin |
| Polyquaternium30 | Ethanaminium, N-(carboxymethyl)-N,N-dimethyl-2-[(2-methyl-1-oxo- |
| | 2-propen-1-yl)oxy]-, inner salt, polymer with methyl 2- |
| | methyl-2-propenoate |
| Polyquaternium31 | N,N-dimethylaminopropyl-N-acrylamidine quaternized with |
| | diethylsulfate bound to a block of polyacrylonitrile |
| Polyquaternium32 | Poly(acrylamide 2-methacryloxyethyltrimethyl ammonium chloride) |
| Polyquaternium33 | Copolymer of trimethylaminoethylacrylate salt and acrylamide |
| Polyquaternium34 | Copolymer of 1,3-dibromopropane and N,N-diethyl-N,N′-dimethyl-1,3- |
| | propanediamine |
| Polyquaternium35 | Methosulphate of the copolymer of |
| | methacryloyloxyethyltrimethylammonium and of |
| | methacryloyloxyethyldimethylacetylammonium |
| Polyquaternium36 | Copolymer of N,N-dimethylaminoethylmethacrylate and |
| | buthylmethacrylate, quaterized with dimethylsulphate |
| Polyquaternium37 | Poly(2-methacryloxyethyltrimethylammonium chloride) |
| Polyquaternium39 | Terpolymer of acrylic acid, acrylamide and diallyldimethylammonium |
| | Chloride |
| Polyquaternium42 | Poly[oxyethylene(dimethylimino)ethylene (dimethylimino)ethylene |
| | dichloride] |
| Polyquaternium43 | Copolymer of acrylamide, acrylamidopropyltrimonium chloride, 2- |
| | amidopropylacrylamide sulfonate and |
| | dimethylaminopropylamine |
| Polyquaternium44 | 3-Methyl-1-vinylimidazolium methyl sulfate-N-vinylpyrrolidone |
| | copolymer |
| Polyquaternium45 | Copolymer of (N-methyl-N-ethoxyglycine)methacrylate and N,N- |
| | dimethylaminoethylmethacrylate, quaternized with dimethyl |
| | sulphate |
| Polyquaternium46 | Terpolymer of vinylcaprolactam, vinylpyrrolidone, and quaternized |
| | vinylimidazole |
| Polyquaternium47 | Terpolymer of acrylic acid, methacrylamidopropyl trimethylammonium |
| | chloride, and methyl acrylate |

in particular derived from poly quaternary compounds based on polysaccharides, preferably based on cellulose, guar gum, chitin and chitosan,
   more preferably quaternized celluloses and crosslinked quaternized celluloses (hydrogel such as quaternized cellulose according to the following structure

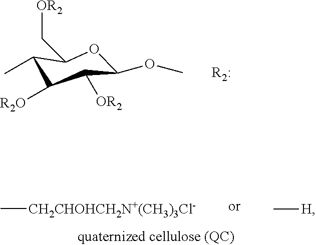

wherein the structure displays a repeating unit of the corresponding polymer,
quaternized cellulose of the following structure

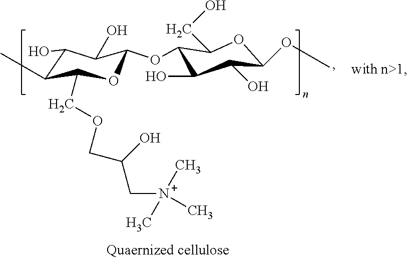

hydrogel of the following structure

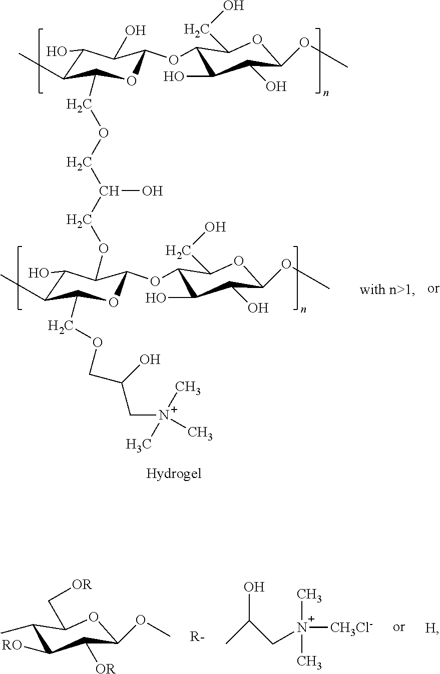

which displays the structure of a repeating unit of the corresponding polymer,
i.e. quaternized hydroxy ethyl celluloses and ethoxylated celluloses, such as

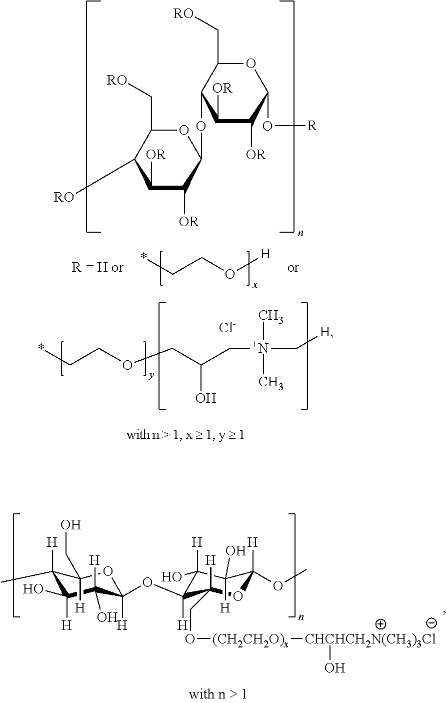

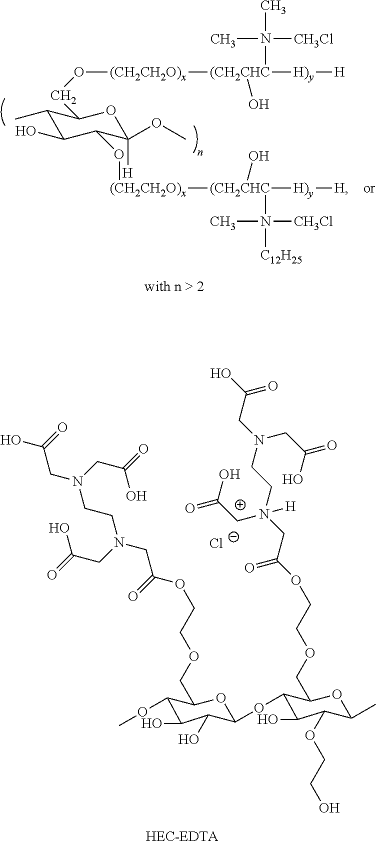

quaternized guar gums, for example of the formula

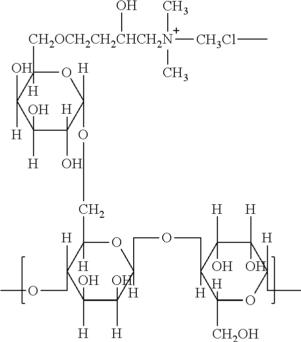

or as obtained in the following reaction scheme

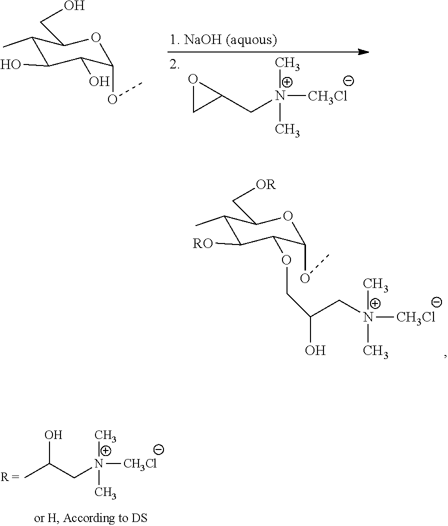

and in particular commercially available guar gum based products such as guar hydroxypropyltrimonium chlorides,
quaternized chitins, such as the products (1) and (2) as obtained in the reaction schemes displayed below,

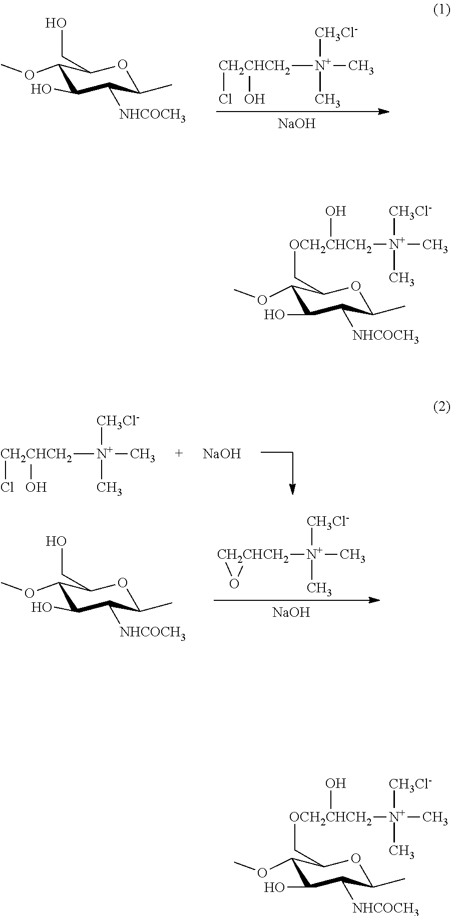

and quaternized chitosans, in particular. N-quaternized chitosans, O-quaternized chitosans, N,O-quaternized chitosans, for example (N-(2-hydroxypropyl)-3-trimethylammonium chitosan chloride (HTCC), Q-chitosan or TMCTPCHT as obtained in the reaction schemes displayed below

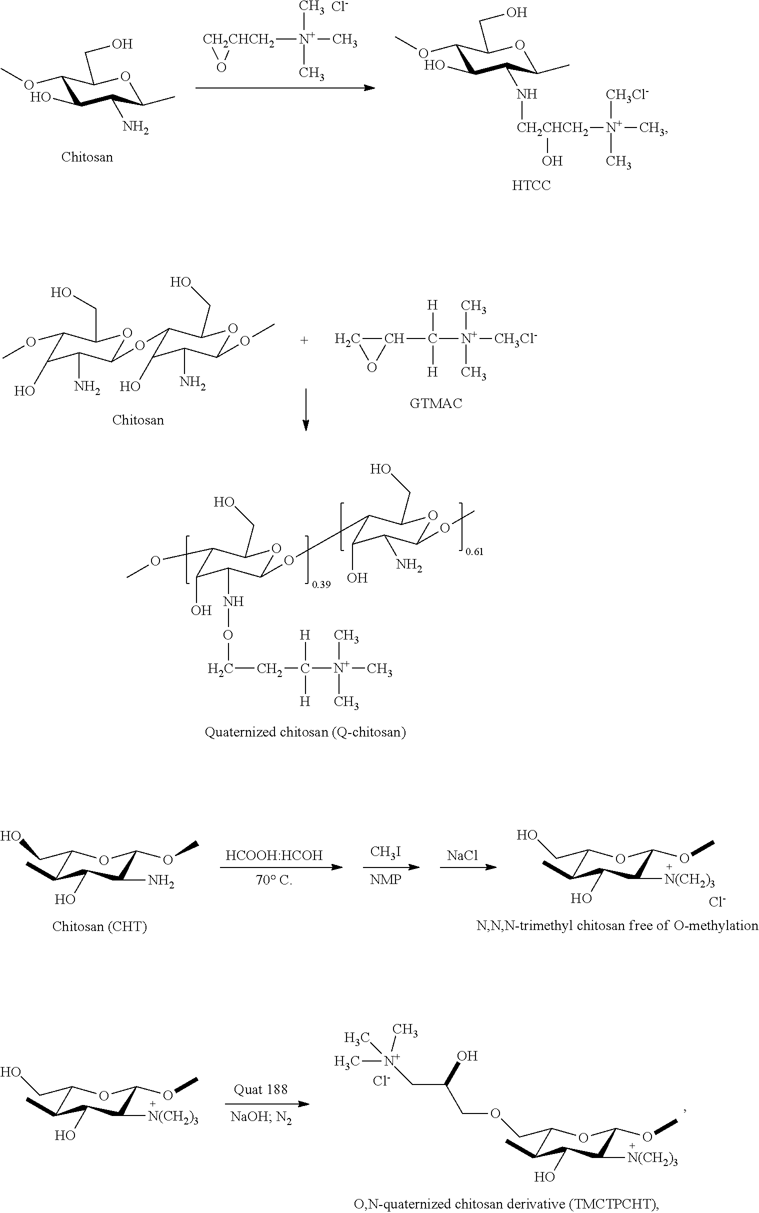

or the structures obtained in the reactions displayed below:

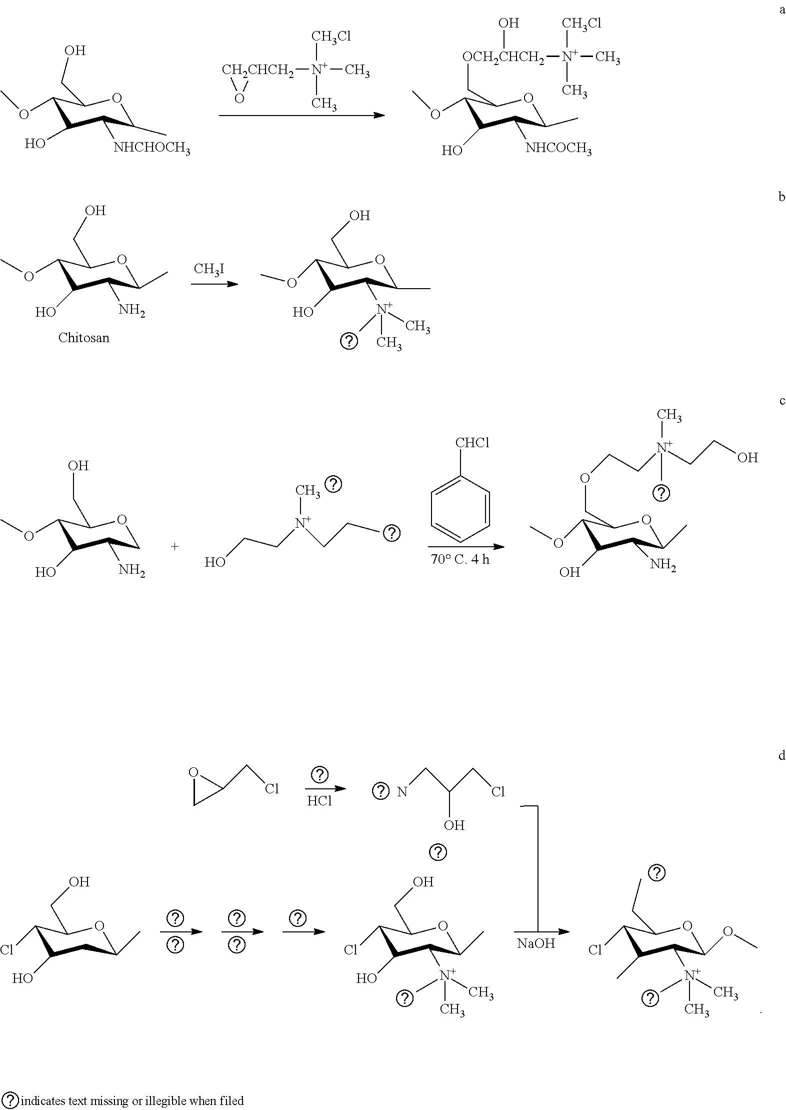

In a preferred embodiment according to the invention, the organic ammonium-group-comprising cation of the organic ammonium salt cation is selected from cations according to the general formula

R¹(—F)ₓ (I) as defined above, wherein R¹ is derived from
monovalent to octadecavalent, preferably divalent to octadecavalent, more preferably divalent to hexavalent, even more preferably divalent, trivalent and tetravalent optionally OH, amino or amido substituted straight-chain, cyclic or branched, saturated, unsaturated or aromatic hydrocarbon groups, derived from alkyl halogenides having more than one, preferred more than two carbon atoms such as alkyl chlorides, bromides, iodides, e.g. 1,3-dichloropropane, 1,3-dichlorobutane, 1,4-dichlorobutane, dichloro-monohydroxy propane isomers, 1,2,3-trichloro propane, 1,2-dichloro hexanediol, 1,2-dichloro hexane, or the respective bromides and iodide derivatives;
monovalent to octadecavalent, preferably divalent to octadecavalent, more preferably divalent to hexavalent, even more preferably divalent, trivalent and tetravalent optionally OH, amino or amido substituted straight-chain, cyclic or branched, saturated, unsaturated or aromatic hydrocarbon groups, derived from esters of halogenated carboxylic acids, preferred, chloro carboxylic acids, in total (ester) having more than two, preferred more than three carbon atoms, such as esters of chloroacetic acid, 3-chloropropionic acid, 4-chlorobutanoic acid or the respective bromo carboxylic acids, with alcohols, in particular methanol, ethanol, 2-propanol, 1-butanol, t-butanol, undec-10-en-ol, oleyl alcohol, stearyl alcohol, 1,2-propanediol, 1,3-propanediol, 1,3-butanediol, 1,4-butanediol, 1,2 hexanediol, 1,6-hexanediol, glycerol, diglycerol, triglycerol and higher linear or branched oligoglycerols, trimethylol propane, castor oil (ricinoleic acid triglyceride), pentaerythritol, sorbitol, poly(alkylene oxides), such as (ethylene oxide)-, (propylene oxide)- and/or (butylene oxide)-based polyethers, e.g. derived from polyethylene glycols, like diethylene glycol, triethylene glycol, tetraethylene glycol, and pentaethylene glycol, or derived from polypropylene glycols, like dipropylene glycol (e.g., derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed (ethylene oxide) and (butylene oxide)-based copolyethers, derived from mixed (propylene oxide)- and (butylene oxide)-based copolyethers, and derived from mixed (ethylene oxide)- and (propylene oxide)- and (butylene oxide)-based copolyethers;
monovalent to octadecavalent, preferably divalent to octadecavalent, more preferably divalent to hexavalent, even more preferably divalent, trivalent and tetravalent optionally OH substituted straight-chain, cyclic or branched, saturated, unsaturated or aromatic hydrocarbon groups, derived from ethers or esters of epoxy compounds, in total having more than three, preferred more than four carbon atoms, preferred glycidyl ethers, by a ring opening reaction with alcohols, in particular methanol, ethanol, 2-propanol, 1-butanol, t-butanol, undec-10-en-ol, oleyl alcohol, stearyl alcohol, 1,2-propanediol, 1,3-propanediol, 1,3-butanediol, 1,4-butanediol, 1,2 hexanediol, 1,6-hexanediol, glycerol, diglycerol, triglycerol and higher linear or branched oligoglycerols, trimethylol propane, castor oil (ricinoleic acid triglyceride), pentaerythritol, sorbitol, poly(alkylene oxide)s, such as (ethylene oxide)-, (propylene oxide)- and/or (butylene oxide)-based polyethers, e.g. derived from polyethylene glycols, like diethylene glycol, triethylene glycol, tetraethylene glycol, and pentaethylene glycol, or derived from polypropylene glycols, like dipropylene glycol (e.g., derived from 2,2′-oxydi-1-propanol, 1,1′-oxydi-2-propanol, and 2-(2-hydroxypropoxy)-1-propanol), tripropylene glycol, tetrapropylene glycol, pentapropylene glycol, derived from mixed (ethylene oxide)- and (butylene oxide)-based copolyethers, derived from mixed (propylene oxide)- and (butylene oxide)-based copolyethers, and derived from mixed (ethylene oxide)- and (propylene oxide)- and (butylene oxide)-based copolyethers, or preferred glycidyl esters, with acids, in particular neodecanoic acid,
monovalent to octadecavalent, preferably divalent to octadecavalent, more preferably divalent to hexavalent, even more preferably divalent, trivalent and tetravalent, optionally OH substituted straight-chain, cyclic or branched, saturated, unsaturated or aromatic hydrocarbon groups, formed from ethers of epoxy compounds, in total having more than seven, preferred more than eight carbon atoms, preferred glycidyl ethers, with di- to hexavalent carboxylic acids, in particular maleic acid, succinic acid, adipic acid, sebacic acid, itaconic acid, tartaric acid, trimellitic acid, fatty dimer acids, carboxyl (—C(O)OH) functionalized polyesters, in particular preferably formed by the condensation of di- to hexavalent carboxylic acids, e.g. maleic acid, succinic acid, adipic acid, sebacic acid, itaconic acid, tartaric acid, trimellitic acid, fatty dimer acids, with di- to hexavalent alcohols as outlined above or alkylene oxides, such as ethylene oxide, propylene oxide, butylene oxide, and compounds comprising at least one glycidoxy group, such as glycidol, diglycidyl ether, glycerol diglycidyl ether, glycerol triglycidyl ether and oligomeric glycerol glycidyl ethers, butanediol diglycidylether, in particular the condensation products of succinic acid, maleic acid and tartaric acid, fatty dimer acids with glycerol diglycidyl ether, polyesters, in particular preferably derived from oligomerized hydroxycarboxylic acids, in particular oligomerized lactic acid, 12-hydroxy stearic acid, lesquerolic acid, ricinoleic acid,
monovalent to octadecavalent, preferably divalent to octadecavalent, more preferably divalent to hexavalent, even more preferably divalent, trivalent and tetravalent, optionally OH substituted straight-chain, cyclic or branched, saturated, unsaturated or aromatic hydrocarbon groups,
   derived from esters of halogenated carboxylic acids, preferably chloro carboxylic acids, in total having more than five, preferred more than six carbon atoms such as esters of chloroacetic acid, 3-chloropropionic acid, 4-chlorobutanoic acid or the respective bromo carboxylic acids, with OH functionalized polyesters, in particular preferably formed by the condensation of di- to hexavalent carboxylic acids, e.g. maleic acid, succinic acid, adipic acid, sebacic acid, itaconic acid, tartaric acid, trimellitic acid, fatty dimer acids, with di- to hexavalent alcohols as outlined above or alkylene oxides, such as ethylene oxide, propylene oxide, butylene oxide, and compounds comprising at least one glycidoxy group, such as glycidol, diglycidyl ether, glycerol diglycidyl ether, glycerol triglycidyl ether and oligomeric glycerol glycidyl ethers, butanediol diglycidylether, in particular the condensation products of succinic acid, maleic acid and tartaric acid or fatty dimer acids with glycerol diglycidyl ether.

In a further preferred embodiment of the invention, the organic ammonium-group-comprising cation is selected from cations according to the general formula

R¹(—F)ₓ (I)

as defined above, wherein R¹ is selected from poly(alkylene oxide) groups,
preferably poly(alkylene oxide) groups of the general formula (XIII):

—[CH₂CH₂O]_{q1}—[CH₂CH(CH₃)O]ᵣ₁—[CH₂CH(C₂H₅)O]ₛ₁—{[CH₂CH₂]_{q2}—[CH₂CH(CH₃)]ᵣ₂—[CH₂CH(C₂H₅)]ₛ₂}— (XIII)

with
q1=0 to 49, preferred 0 to 10, more preferred 1 to 10, even more preferred 1 to 5,
r1=0 to 32, preferred 0 to 10, more preferred 1 to 10, even more preferred 1 to 5,
s1=0 to 24, preferred 0 to 10, more preferred 1 to 10, even more preferred 1 to 5,
q2=0 or 1,
r2=0 or 1,
s2=0 or 1, and
Σ(q2+r2+s2)=1,
with the proviso that the sum of the carbon atoms in such poly(alkylene oxide) groups is 2 to 100, preferred 2 to 50, more preferred 2 to 30, even more preferred 2 to 20, specific 2 to 15, or
R¹ is selected from divalent hydrocarbon groups derived from oligoglycerols of the general formula (XIV):

—[CH₂CH(R¹²)CH₂O]ₜ₁—[CH₂CH(R¹²)CH₂)]ₜ₂— (XIV)

with
t1=0 to 32, preferred 0 to 10, more preferred 1 to 10, even more preferred 1 to 5, specifically 1 and 2,
t2=1,
R¹²=OH or —O—C(O)—R⁶—N⁺(R³, R⁴, R⁵),
wherein R³, R⁴, R⁵ and R⁶ are as defined above,
with the proviso that the sum of the carbon atoms is 2 to 100, preferred 2 to 50, more preferred 2 to 30, even more preferred 2 to 20, specific 2 to 15,
or R¹ is selected from
divalent hydrocarbon groups, comprising at least one ester group of the general formula (XV):

—[CH₂CH₂O]_{q1}—R¹³—[CH₂CH₂O]_{q1}—[CH₂CH₂]_{q2}— (XV)

with q1 being the same or different and being as defined above and q2=1
and of the formula (XVI)

—[CH₂CH(R¹²)CH₂O]ₜ₁—R¹³—[CH₂C H(R¹²)CH₂O]ₜ₁—[CH₂CH(R¹²)CH₂)]ₜ₂— (XVI)

with t1, t2 and R¹² as defined above, and
R¹³ being selected from —C(O)C(O)O—, —C(O)(CH₂)₁₋₈C(O)O—, such as being derived from succinic acid, adipic acid, sebacic acid, or —C(O)(C₆H₄)C(O)O—, i.e. derived from phthalic and terephthalic acid, —C(O)CH═CHC(O)O—, —C(O)C(═CH₂)—CH₂C(O)O—, —C(O)CH(OH)CH(OH)C(O)O—,
with the proviso that the sum of the carbon atoms in R¹³ is 2 to 100, preferred 2 to 50, more preferred 2 to 30, even more preferred 2 to 20, specifically 2 to 15,
preferably, q2=0, and one or two of q1, r1 and s1 are 0, and more preferably
q2=0, r1 and s1 are 0, or
q2=0, q1 and s1 are 0,
or
R¹ is a group that contains one or more groups —O—, such as one to five and these groups —O— are preferably ether groups but can also form an ester group together with a carbonyl group, and preferably the group R¹ is substituted by one or more hydroxyl groups.

In a preferred embodiment of the invention, in at least one of the moieties of the formula (VI), (VI*) or (VIII), (VIII*) of the carboxylate anion (COO⁻) group-comprising anion of the formula (V) or (VII) of the organic ammonium salt according to the invention two or more different R⁸ groups are present.

The presence of at least two different groups R⁸ in the moieties of the formula (VI), (VI*) or (VIII), (VIII*) results when at least two different types of hydroxy-substituted or amino-substituted carboxylic acid derivatives are used in the preparation of these chain structures. The different groups R⁸ may differ from each other in the number of C atoms, but also with regard to the number and position of double bonds, if any, and/or the position of substituents and the position of the linkage to the adjacent groups. They may differ with regard to if they are linear or branched.

It is preferred when in at least one group G or Y R⁸ independently represents hydrocarbon groups derived from ricinoleic acid and 12-hydroxy stearic acid.

In a further preferred embodiment of the invention, in at least one of the moieties of the formulas (VI), (VI*) or (VIII), (VIII*) of the carboxylate anion (COO⁻) group-comprising anion of the formulas (V) or (VII) of the organic ammonium salt according to the invention the groups R⁸ and R⁹ or R⁸ and R^{9*} are not based on the same carboxylic acid structure.

Preferably, all moieties of the formula (VI), (VI*), (VIII) or (VIII*) present in the anions of the organic ammonium salt are terminated by a group R⁹ or R^{9*} different from the groups R⁸ constituting the internal members of the estolide chain structures.

It is further preferred that R⁸ and R⁹ or R⁸ and R^{9*}, respectively, differ from each other regarding their number of carbon atoms, the number or position of double bonds, if any, in the carbon chain, or regarding the position of oxygen or nitrogen atoms bonded to the carbon chain of the groups. The carboxylic acid structures from which said groups are derived may also differ by two or more of the above-mentioned features.

In another preferred embodiment of the invention, in the carboxylate anion (COO⁻) group-comprising anion of the formula (V) of the organic ammonium salt according to the invention

p is 2-6,
R⁷ is selected from di- to hexavalent linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 1000 carbon atoms, more particular 1 to 150 carbon atoms,
and at least one group G contains one or more moieties of the general formula (VI*)

—R⁸(—X—C(O)—R⁸)ₘ—X—C(O)—R⁹* (VI*)

wherein R⁸, R^{9*} and m are as defined above, or
in the carboxylate anion (COO⁻) group-comprising anion of the formula (VII) of the organic ammonium salt q is 2-6,
R⁷ is selected from di- to hexavalent linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups, for instance phenylene, benzylene or tolylene groups, in particular from such groups having 1 to 1000 carbon atoms, more particular 1 to 150 carbon atoms,
and at least one group Y contains one or more moieties of the general formula (VIII*)

—R⁸(—C(O)—X—R⁸)ₘ—C(O)—X—R^{9*} (VIII*)

wherein R⁸, R⁹* and m are as defined above.

According to this embodiment, it is preferred that in formula (V) p is 2, 3 or 4, most preferably p is 2, or it is preferred that in formula (VII) q=2, 3 or 4, most preferably q is 2.

It is also preferred that preferred that in the compound of the general formula (V) R⁷ is selected from linear, branched or cyclic alkylene groups having 1 to 150 carbon atoms, more preferably linear alkylene groups having 1 to 12 carbon atoms, or it is preferred that in formula (VII) R⁷ is selected from linear, branched or cyclic alkylene groups having 1 to 150 carbon atoms, more preferably linear alkylene groups having 1 to 12 carbon atoms.

In a further preferred embodiment of the invention, in the carboxylate anion (COO⁻) group-comprising anion of the formula (V) of the organic ammonium salt according to the invention

### X=O,

p is 2,
R⁷ is selected from divalent linear, branched and cyclic alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups, preferably from ethylene, n-propylene, n-butylene, n-pentylene and n-hexylene,
and at least one group G contains one or more moieties of the general formula (VI*)

—R⁸(—X—C(O)—R⁸)ₘ—X—C(O)—R^{9*} (VI*)

wherein R⁸, R^{9*} and m are as defined above,
or in the carboxylate anion (COO⁻) group-comprising anion of the formula (VII)

### X=O,

q is 2,
R⁷ is selected from divalent linear, branched and cyclic alkylene groups, in particular from linear C1-C22 alkyl groups such as methylene, ethylene, n-propylene, n-butylene, n-pentylene, n-hexylene, n-heptylene or n-octylene groups, branched C1-C22 alkylene groups iso-propylene, iso-butylene, tert-butylene, iso-butylene, tert-pentylene, neo-pentylene, and 2-ethylhexylene groups, preferably from ethylene, n-propylene, n-butylene, n-pentylene and n-hexylene,
and at least one group Y contains one or more moieties of the general formula (VIII*)

—R⁸(—C(O)—X—R⁸)ₘ—C(O)—X—R^{9*} (VIII*)

wherein R⁸, R^{9*} and m are as defined above.

Preferably, R⁸ is derived from lactic acid, ricinoleic acid, lesquerolic acid, 10-hydroxy stearic acid, 12-hydroxy stearic acid, 14-hydroxy tetradecanoic acid, most preferably from ricinoleic acid or lesquerolic acid.

In a specifically preferred embodiment according to the invention, in a carboxylate anion (COO⁻) group-comprising anion of the formula (V) containing one or more moieties of the general formula (VI*)

—R⁸(—X—C(O)—R⁸)ₘ—X—C(O)—R⁹* (VI*)

wherein R⁸, R^{9*} and m are as defined above,
or in a carboxylate anion (COO⁻) group-comprising anion of the formula (VII) containing one or more moieties of the general formula (VIII*)

—R⁸(—C(O)—X—R⁸)ₘ—C(O)—X—R^{9*} (VIII*)

wherein R⁸, R^{9*} and m are as defined above,
one or more groups R^{9*} are each terminated by three or more groups —O—C(O)-T, preferably each by 4 or more groups —O—C(O)-T, most preferably each by 4 to 12 groups —O—C(O)-T, wherein T is as defined above.

Therein, it is preferred that branched structures of R^{9*} containing a branching structure are terminated by 3 to 10 groups —O—C(O)-T, while dendrimeric structures containing at least two branching structures are preferably terminated by 4 to 20 groups —O—C(O)-T.

In another preferred embodiment according to the invention, in the carboxylate anion (COO⁻) group-comprising anion of the formula (V) containing one or more moieties of the general formula (VI*)

—R⁸(—X—C(O)—R⁸)ₘ—X—C(O)—R⁹* (VI*)

wherein R⁸, R^{9*} and m are as defined above, or (VII) containing one or more moieties of the general formula (VIII*)

—R⁸(—C(O)—X—R⁸)ₘ—C(O)—X—R^{9*} (VIII*)
wherein R⁸, R^{9*} and m are as defined above,
   one or more groups R^{9*} each contain at least two branching structures of the general formula

—C(O)—B(—O—)_{b},

wherein B is a linear or branched hydrocarbon group having 2-20 carbon atoms, and b is 2 or more, and wherein the b groups (—O—) linked to the group B on the one side are linked to a C atom which may be the C atom of a CH₂ group or of a carbonyl group on the other side.

While the presence of a branching structure of the general formula

—B(—O—)_{b}

as defined above is mandatory in the group R^{9*} in order to enable a branched structure which may be terminated by two or more groups —O—C(O)-T, the presence of two or more further branching structures of the general formula

—C(O)—B(—O—)_{b}

as defined above results in the formation of a dendrimeric structure, i.e. a structure having several branching points which may be arranged consecutively or parallel when moving from the bond linking R^{9*} to the rest of the molecule to the terminal groups of R^{9*}. However, deviating from the IUPAC definition of a dendrimer molecule [see A. Fradet et al., Pure and Applied Chemistry, 91(3), 523-561: Nomenclature and terminology for dendrimers with regular dendrons and for hyperbranched polymers (IUPAC Recommendations 2017)] the dendrons do not have to comprise exclusively dendritic and terminal constitutional repeating units, and it is not required that each path from the free valence of R^{9*}, i.e. the valence bonding R^{9*} to the rest of the molecule, to any end-group comprises the same number of constitutional repeating units.

It is further preferred that all (—O—) groups of the branching structure of the general formula —B(—O—)_{b} as defined above are substituted by the branching structures of the general formula —C(O)—B(—O—)_{b} as defined above.

It is also preferred that one or more groups R⁹* contain 3 or more branching structures —C(O)—B(—O—)_{b}, more preferred 3-5 of said branching structures.

In this embodiment, it is preferred that b for both branching structures is independently selected from the range of 2-6, more preferably from the range of 2-4.

In a further preferred embodiment according to the invention, in the carboxylate anion (COO⁻) group-comprising anion of the formula (V) or (VII) as defined in the previous embodiment in one or more groups R^{9*} the one or more branching structures of the general formal —B(—O—)_{b} or —C(O)—B(—O—)_{b} as defined above are independently derived from glyceric acid, 2,2-di-hydroxymethyl propionic acid, gluconic acid, maltobionic acid, lactobionic acid.

It is preferred that all branching structures present in a group R^{9*} are independently derived from 2,2-di-hydroxymethyl propionic acid, more preferably all branching structures in at least one group R^{9*} are derived from 2,2-di-hydroxymethyl propionic acid.

It is even further preferred that all branching structure present in all groups R^{9*} of a compound of the formula (V) or (VII) are derived from the same polyhydroxy carboxylic acid.

In another preferred embodiment according to the invention, in the carboxylate anion (COO⁻) group-comprising anion of the formula (V) containing one or more moieties of the general formula (VI*)

—R⁸(—X—C(O)—R⁸)ₘ—X—C(O)—R⁹* (VI*)

wherein R⁸, R⁹* and m are as defined above,
or of the formula (VII) containing one or more moieties of the general formula (VIII*)

—R⁸(—C(O)—X—R⁸)ₘ—C(O)—X—R^{9*} (VIII*)

wherein R⁸, R^{9*} and m are as defined above,
one or more groups R^{9*} are each terminated by two or more groups of the structure

—R⁸(—X—C(O)—R⁸)ₜ—X—C(O)-T,

wherein R⁸, and T are as defined above, and

### X=O,

t is independently 0-12, preferably t is independently 0-6, most preferably t is independently 0, 1, 2 or 3,

In this embodiment, two or more of the terminal groups as defined above are positioned at the terminus of an estollde chain. It is preferred that the one or more groups R^{9*} are each terminated by 2-48 groups of the general formula —R⁸(—X—C(O)—R⁸)ᵣ—X—C(O)-T, more preferable by 2-27 groups of the formula —R⁸(—X—C(O)—R⁸)ₜ—X—C(O)-T, and most preferable by 4-16 groups of the formula —R⁸(—X—C(O)—R⁸)ₜ—X—C(O)-T.

In still another preferred embodiment according to the invention, in the carboxylate anion (COO⁻) group-comprising anion of the formula (V)

containing one or more moieties of the general formula (VI*)

—R⁸(—X—C(O)—R⁸)ₘ—X—C(O)—R⁹* (VI*)

wherein R⁸, R^{9′} and m are as defined above, or in the carboxylate anion (COO⁻) group-comprising anion of the formula (VII) containing one or more moieties of the general formula (VIII*)

—R⁸(—C(O)—X—R⁸)ₘ—C(O)—X—R^{9*} (VIII*)

wherein R⁸, R^{9*} and m are as defined above,
one or more groups R⁹* are terminated by two or more groups, preferably 4 to 12 groups of the structure

—R⁸(—X—C(O)—R⁸)ₜ—X—C(O)-T,

wherein R⁸ is independently derived from C8-C24 monocarboxy-monohydroxy carboxylic acids, in particular ricinoleic acid, 12-hydroxy stearic acid, lesquerolic acid, 11-hydroxy-undecanoic acid,

### X is O, and

T is independently derived from C2 to C24, preferred C8 to C24 fatty acids, in particular lauric acid, myristic acid, palmitic acid, oleic acid, stearic acid, behenic acid, arachidic acid,
and t is 0-6, preferably 0, 1, 2 or 3,

According to this embodiment it is preferred when R⁸ is derived from ricinoleic acid, and T is derived from stearic acid or oleic acid.

More preferably, the R⁸ of all groups R^{9*} are derived from ricinoleic acid, and even more preferably in all groups R^{9′} R⁵ is derived from ricinoleic acid, T is derived from stearic acid or oleic acid, and t is 0, 1, 2 or 3.

In a further preferred embodiment according to the invention, in the carboxylate anion (COO⁻) group-comprising anion of the formula (V)

containing one or more moieties of the general formula (VI*)

—R⁸(—X—C(O)—R⁸)ₘ—X—C(O)—R⁹* (VI*)

wherein R⁸, R^{9*} and m are as defined above, or in the carboxylate anion (COO⁻) group-comprising anion of the formula (VII) containing one or more moieties of the general formula (VIII*)

—R⁸(—C(O)—X—R⁸)ₘ—C(O)—X—R^{9*} (VIII*)

wherein R⁸, R^{9*} and m are as defined above,
one or more groups R⁹* are independently selected from one of the following branched or dendrimeric fatty acid structures:

—R¹⁴—O—C(O)—R¹⁵—(O—C(O)—R¹⁶)ₘ₁—(O—C(O)—R¹⁷)ₘ₂—O—C(O)-T or

—R¹⁴—NR¹⁰—C(O)—R¹⁵—(O—C(O)—R¹⁶)ₘ₁—(O—C(O)—R¹⁷)ₘ₂—O—C(O)-T,

wherein
R¹⁰ is as defined above,
R¹⁴ is selected from divalent optionally substituted hydrocarbon radicals which 2 to and 50 carbon atoms, specifically 2 to 20 carbon atoms, more specifically 2 to 10 carbon atoms and may contain optionally one or more groups selected from —O—, —NH—, —C(O)—, —C(S)—, tertiary amino groups

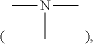

and can be substituted by —OH or halide groups, wherein the radical R¹⁴ cannot contain a combination of a —C(O)— group and a —O— group or a combination of a —C(O)— group and a —NH— or tertiary amino group forming an internal carboxylate group or an internal amide group, and preferably represents C1-C24 n-alkylene groups and CC2-C24 n-alkenylene groups, in particular-CH₂—, —CH₂CH₂—, —CH₂CH₂CH₂—,
R¹⁵ is independently selected from a divalent optionally substituted straight-chain, cyclic or branched, saturated or unsaturated hydrocarbon radical which have up to 36 carbon atoms or from a C2 to C24 monocarboxylic acid having 2 to 6 hydroxy groups, for example 2,2′-di-hydroxymethyl propanoic acid,
m1 is 0 to 12, preferred 0 to 10, more preferred 0 to 6, even more preferred 1 to 6, specifically 0, 1, 2, 3, 4, 5, 6,
m2 is 0 to 12, preferred 0 to 10, more preferred 0 to 6, even more preferred 1 to 6, specifically 0, 1, 2, 3, 4, 5, 6,
and m1+m2 is t, wherein t is 0 to 12, preferred 0 to 10, more preferred 0 to 6, even more preferred 1 to 6, specifically 0, 1, 2, 3, 4, 5, 6 and
T is as defined above,
R¹⁶ and R¹⁷ are selected from the groups R¹⁵ as defined above,
and wherein preferably

| | | R¹⁷ adjacent to | |
|---|---|---|---|
| R¹⁵ adjacent to R¹⁴ is | R¹⁶ adjacent to R¹⁵ is | O—C(O)—T is derived | |
| derived from | derived from | from | T is derived from |
| di- or polyhydroxylated | unsaturated acid, in | unsaturated acid, in | unsaturated acid, in |
| acid, in particular | particular ricinoleic | particular ricinoleic | particular oleic acid |
| 2,2′-di-hydroxymethyl | acid | acid | |
| propanoic acid | | | |
| di- or polyhydroxylated | unsaturated acid, in | unsaturated acid, in | saturated acid, in |
| acid, in particular | particular ricinoleic | particular ricinoleic | particular octadecanoic |
| 2,2′-di-hydroxymethyl | acid | acid | acid, neodecanoic acid |
| propanoic acid | | | |
| di- or polyhydroxylated | unsaturated acid, in | saturated acid, in | saturated acid, in |
| acid, in particular | particular ricinoleic | particular 12- | particular octadecanoic |
| 2,2′-di-hydroxymethyl | acid | hydroxystearic acid | acid, neodecanoic acid |
| propanoic acid | | | |
| di- or polyhydroxylated | saturated acid, in | saturated acid, in | saturated acid, in |
| acid, in particular | particular 12- | particular 12- | particular octadecanoic |
| 2,2′-di-hydroxymethyl | hydroxystearic acid | hydroxystearic acid | acid, neodecanoic acid |
| propanoic acid | | | |
| di- or polyhydroxylated | saturated acid, in | saturated acid, in | unsaturated acid, in |
| acid, in particular | particular 12- | particular 12- | particular oleic acid |
| 2,2′-di-hydroxymethyl | hydroxystearic acid | hydroxystearic acid | |
| propanoic acid | | | |
| di- or polyhydroxylated | saturated acid, in | unsaturated acid, in | unsaturated acid, in |
| acid, in particular | particular 12- | particular ricinoleic | particular oleic acid |
| 2,2′-di-hydroxymethyl | hydroxystearic acid | acid | |
| propanoic acid | | | |
| di- or polyhydroxylated | di- or polyhydroxylated | unsaturated acid, in | unsaturated acid, in |
| acid, in particular | acid, in particular | particular ricinoleic | particular oleic acid |
| 2,2′-di-hydroxymethyl | 2,2′-di-hydroxymethyl | acid | |
| propanoic acid | propanoic acid | | |
| di- or polyhydroxylated | di- or polyhydroxylated | unsaturated acid, in | saturated acid, in |
| acid, in particular | acid, in particular | particular ricinoleic | particular 12- |
| di-hydroxymethyl | 2,2′-di-hydroxymethyl | acid | hydroxystearic acid |
| propanoic acid | propanoic acid | | |
| di- or polyhydroxylated | di- or polyhydroxylated | saturatedacid, in | unsaturated acid, in |
| acid, in particular | acid, in particular | particular 12- | particular oleic acid |
| 2,2′-di-hydroxymethyl | 2,2′-di-hydroxymethyl | hydroxystearic acid | |
| propanoic acid | propanoic acid | | |
| di- or polyhydroxylated | di- or polyhydroxylated | saturated acid, in | saturated acid, in |
| acid, in particular | acid, in particular | particular 12- | particular 12- |
| 2,2′-di-hydroxymethyl | 2,2′-di-hydroxymethyl | hydroxystearic acid | hydroxystearic acid |
| propanoic acid | propanoic acid | | |
| di- or polyhydroxylated | unsaturated acid, in | di- or polyhydroxylated | unsaturated acid, in |
| acid, in particular | particular ricinoleic | acid, in particular | particular oleic acid or |
| 2,2′-di-hydroxymethyl | acid | 2,2′-di-hydroxymethyl | unsaturatedacid |
| propanoic acid | | propanoic acid | terminated oligoester |
| di- or polyhydroxylated | saturated acid, in | di- or polyhydroxylated | saturated acid, in |
| acid, in particular | particular 12- | acid, in particular | particular octadecanoic |
| 2,2′-di-hydroxymethyl | hydroxystearic acid | 2,2′-di-hydroxymethyl | acid, neodecanoic acid |
| propanoic acid\| | | propanoic acid | or saturated acid |
| | | | terminated oligoester |

wherein the total number of carbon atoms in R¹⁵+T (Σ carbon atoms R¹⁵, T) is 19 to 300, preferred 25 to 300, more preferred 35 to 300, even more preferred 50 to 300, specifically 35 to 200, more specifically 35 to 150, even more specifically 50 to 150,
with the proviso that for R¹⁵, R¹⁹ and R¹⁷ being derived from di- or polyhydroxylated carboxylic acids at least one, preferred one to two, more preferred two, even more preferred all OH groups are esterified.

According to this embodiment, it is also preferred that one or more groups R⁹* of the anion of the formula (V) or (VII) are independently derived from branched or dendrimeric fatty acid structures obtained by the esterification of 2,2′-di-hydroxymethyl propanoic acid with di-hydroxymethyl propanoic acid itself, C2 to C24, preferred C8 to C24 fatty acids, further preferred lauric acid, myristic acid, palmitic acid, oleic acid, stearic acid, behenic acid, arachidic acid and optionally mono hydroxy fatty acids, in particular ricinoleic acid, as exemplified by the structural formula

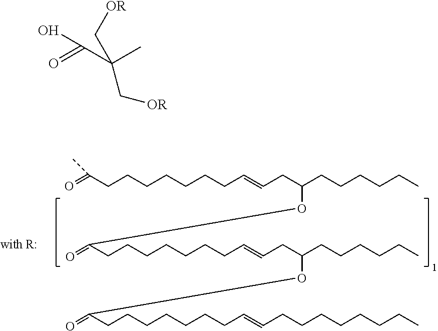

or the structure

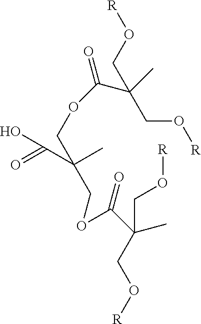

with R as displayed above.

Preferably, all groups R^{9*} of the anion of the formula (V) or (VII) are independently selected from the above-cited group of structures.

Even more preferably, all groups R^{9*} of the anion of the formula (V) or (VII) are represented by a single formula selected from the above-cited group of structures.

It is herewith clarified that the branched or dendrimeric fatty acid structures:

—R¹⁴—O—C(O)—R¹⁵—(O—C(O)—R¹⁶)ₘ₁—(O—C(O)—R¹⁷)ₘ₂—O—C(O)-T or

—R¹⁴—NR¹⁰—C(O)—R¹⁵—(O—C(O)—R¹⁶)ₘ₁—(O—C(O)—R¹⁷)ₘ₂—O—C(O)-T

do not display the substitution of all OH groups of a branching structure which may be displayed by, but reflect the sequence of building blocks required to fulfill the structural requirements of this embodiment.

In a preferred embodiment of the invention, the carboxylate anion (COO—) group-comprising anion of the formula (V) of the organic ammonium salt is represented by the following schematic ester structure:

⁻O(O)C—(C1-C12 radical)-C(O)—O-(mono or oligo C8-C24 hydroxy fatty acid)-C(O)—O—(C2-C10 hydrocarbon)-O—C(O)-(mono or oligo C8-C24 hydroxy fatty acid)-O—C(O)—(C1-C12 radical)-C(O)O⁻,

wherein
the terminal C1-C12 radicals are selected from linear, branched, saturated, unsaturated or aromatic C1-C12 radicals, preferably from C2-C12, C2-C10, C2-C4 radicals, more preferably C2 and C4 radicals.

It is preferred that the (C1-C12 radicals and the adjacent —C(O)— group and the terminal carboxylate groups are derived from the respective acids such as malonic acid, succinic acid, adipic acid, sebacic acid, maleic acid, phthalic acid, terephthalic acid or their anhydrides, such as succinic anhydride, maleic anhydride and phthalic anhydride.

For instance, a terminal group of the formula

—O(O)C—(C₂H₄)—C(O)—

is either derived from succinic acid or succinic anhydride.

It is particularly preferred that both terminal groups of the structure are derived from the same type of dicarboxylic acid or dicarboxylic acid anhydride.

It is noted that in the schematic ester structure according to this embodiment the term “mono or oligo C8-C24 hydroxy fatty acid”

refers to mono hydroxy fatty acids or oligomers of hydroxy fatty acids of the same or different type of hydroxy fatty acids obtained by esterification, wherein the group “—O—” linking the moiety to the terminal group “O(O)C—(C1-C12 radical)-C(O)—” and the group “—C(O)—(O)—” linking the group to the central diol moiety are formally abstracted in the schematic ester structure although they are derived from the terminal —OH and the terminal C(O)OH moieties of the mono or oligo hydroxy fatty acid group.

In a further preferred embodiment according to the invention, the carboxylate anion (COO—) group-comprising anion of the formula (V) or (VII) comprises

at least one moiety of the general formula

—([—O—C(O)—R⁸(—O—C(O)—R⁸)l—O—C(O)-L-C(O)—O—(R⁸—C(O)—O)l—R⁸—C(O)O])—

wherein R⁸ is as defined above,
is an integer independently selected from 0-20, more preferably from 1-12, even more preferably from 2 to 10, and
L is a divalent hydrocarbon radical which may have 1 to 30 carbon atoms and may contain optionally one or more groups selected from —O—, —S—, —C(O)—, —C(S)—, tertiary amino groups

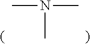

and quaternary ammonium groups,
preferably L is a divalent alkylene or alkenylene radical having 1 to 30 carbon atoms, more preferably L is selected from methylene, ethylene, propylene, butylene, pentylene, hexylene, heptylene, octylene, nonylene, ethenylene, propenylene, butenylene, pentenylene, hexenylene, heptenylene, octenylene, nonenylene,
most preferably L is selected from methylene, ethylene, ethenylene or butenylene.

Preferably the anion according to this embodiment contains one moiety of the general formula

(—([—O—C(O)—R⁸(—O—C(O)—R⁸)l—O—C(O)-L-C(O)—O—(R⁸—C(O)—O)l—R⁸—C(O)O])—

as described above.

It is explicitly noted that there may be an overlap with other moieties as described herein, for example an overlap of the structures of the moiety of the formula (VI)

—R⁸(—C(O)—X—R⁸)ₘ—C(O)—X—R⁹ (VI)

and of the general formula

(—[—O—C(O)—R⁸(—O—C(O)—R⁸)ₗ—O—C(O)-L-C(O)—O—(R⁸—C(O)—O)ₗ—R⁸—C(O)O])—

as defined above.

According to the invention, the presence of a structure of the general formula

—([—O—C(O)—R⁸(—O—C(O)—R⁸)ₗ—O—C(O)-L-C(O)—O—(R⁸—C(O)—O)ₗ—R⁸—C(O)O])—R⁹

fulfills the requirements of the radical G of Formula (V).

In a further preferred embodiment according to the invention, the carboxylate anion (COO—) group-comprising anion of the formula (V) or (VII) comprises at least one moiety of the general formula

—([—O—C(O)—R⁸(—O—C(O)—R⁸)l—O—C(O)-L-C(O)—O—(R⁸—C(O)—O)l—R⁸—C(O)O])—

wherein L and l are as defined above in the previous embodiment,
and R⁸ is independently derived from C8-C24 monocarboxy-monohydroxy carboxylic acids, in particular from ricinoleic acid, 12-hydroxy stearic acid, lesquerolic acid, 11-hydroxy-undecanoic acid,
most preferably R⁸ is derived from ricinoleic acid.

Preferably, L is selected from C1 to C10, preferably methylene, ethylene, butylene, octylene, decylene, l is independently in the range of 0 to 4, and R⁸ is derived from ricinoleic acid. In another preferred embodiment according to the invention, the carboxylate anion (COO—) group-comprising anion of the formula (V) or (VII) comprises at least one moiety of the following structure:

(fatty alcohol)-O—C(O)-(mono or oligo C8-C24 hydroxy fatty acid)-O—C(O)—(C2-C12 hydrocarbon)-C(O)—O-(mono or oligo C8-C24 hydroxy fatty acid)-C(O)—O-(fatty alcohol),

Therein, a C2-C12 hydrocarbon is a C2-C12 hydrocarbylene group, in particular derived from succinic acid, maleic acid, itaconic acid, adipic acid, sebacic acid, dodecanedioic acid, a mono or oligo C8-C24 hydroxy fatty acid is a group derived from a C8-C24 hydroxy-substituted carboxylic acid monomer or an oligomer of up to 20 C8-C24 hydroxy-substituted carboxylic acid monomers formed via esterification, in particular derived from mono or oligo ricinoleic acid with a degree of oligomerization of 2 to 20, preferred, 2 to 10, more preferred 2 to 6, even more preferred 2 to 4, and a fatty alcohol is a group derived from C2 to C24, preferred C8 to C24 fatty alcohols, in particular from n-octanol, n-decanol, n-dodecanol, n-tetradecanol, n-hexacedanol, oleyl alcohol, stearyl alcohol, behenyl alcohol, arachidyl alcohol.

Such kind of compound is exemplified by the following structure:

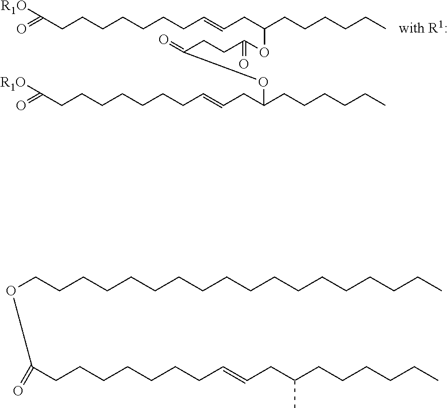

(The dashed bond indicates R¹'s bond to the O atom of the structure above R¹.)

In a further preferred embodiment according to the invention the carboxylate anion (COO—) group-comprising anion of the formula (V) or (VII) comprises at least one moiety of the structure

—([—O—C(O)—R⁸(—O—C(O)—R⁸)ₗ—O—C(O)-L-C(O)—O—(R⁸—C(O)—O)ₗ—R⁸—C(O)O])—R⁹,

wherein L, l, R⁸ and R⁹ are as defined above.

Preferably, L, l and R⁸ are as defined above, and R⁹ is selected from C1-C23 hydrocarbyl groups, preferably C1-C18 hydrocarbyl groups, more preferably C7-C19 hydrocarbyl groups, even more preferably C11-C17 hydrocarbyl groups, most preferably derived from lauric acid, myristic acid, palmitic acid, oleic acid, and stearic acid.

Further preferably, the compound of the formula (V) has the following structure

R⁹—([—O—C(O)—R⁸(—O—C(O)—R⁸)ₗ—O—C(O)-L-C(O)—O—(R⁸—C(O)—O)ₗ—R⁸—C(O)O])—R⁹,

wherein L, l and R⁸ are as defined above, and R⁹ is independently selected from C1-C23 hydrocarbyl groups, preferably C1-C18 hydrocarbyl groups, more preferably C7-C19 hydrocarbyl groups, even more preferably C11-C17 hydrocarbyl groups, and most preferably derived from lauric acid, myristic acid, palmitic acid, oleic acid, and stearic acid.

Even further preferably, L is selected from methylene, ethylene or ethenylene, butylene, hexylene, octylene, decylene or derived from itaconic acid,

l is independently selected from the range of 0-4,
R⁸ is selected derived from ricinoleic acid, and
R⁹ is selected derived from oleic acid or stearic acid.

In a still further preferred embodiment of the invention, the carboxylate anion (COO—) group-comprising anion of the formula (V) or (VII) contains at least one moiety of the formula

R^{1*}[(—O—C(O)—R⁸)ₘ—O—C(O)—]₂,

wherein R^{1*} is a divalent C1-C100 radical, preferably a C1-C12 alkylene, most preferably a methylene, ethylene, 1,3-propylene, 1,4-butylene, 1,6-hexylene, 1,2-propylene, 1,3-butylene radical,
m is independently selected from 1 to 12, and
R⁸ is as defined above.

Therein, the moieties of the formula

R^{1*}[(—O—C(O)—R⁸)ₘ—O—C(O)—]₂,

are preferably formed starting from alkylene diols, more preferably from α,ω-alkylene diols such as 1,2-ethane diol, 1,3-propane diol, 1,4-butane diol and 1,6 hexanediol, by sequential or blockwise ester chain formation.

Regardless whether single hydroxy-substituted carboxylic acids are added in an iterative manner or if estolide chains with a carboxylic acid group are brought to reaction with a such diol, using an excess of the carboxylic acid reactant results in the formation of a product in which the diol is predominantly esterified in the same manner on both ends, i.e. a symmetrical structure of the formula

R^{1*}[(—O—C(O)—R⁸)ₘ—O—C(O)—]₂

is thus obtained.

The structure of the group R^{1*} thus usually corresponds directly to the alkylene diols applied as starting materials.

According to the invention, R^{1*} is divalent C2-C100 hydrocarbon radical, which includes all types of linear, branched and cyclic aliphatic and aromatic divalent hydrocarbon groups, such as alkylenes, alkenylenes, alkynylenes as well as aromatic structures, such as phenylenes.

As C1-C12 alkylene diols are preferred starting materials, R^{1*} is preferably a C1-12 alkylene group, more preferably a methylene, ethylene, n-propylene, n-butylene, n-pentylene, n-hexylene, even more preferably a methylene, ethylene, n-propylene or n-butylene or n-hexylene group.

While according to this embodiment m is independently selected, it is preferred that both m of the general structure

R^{1*}[(—O—C(O)—R⁸)ₘ—O—C(O)—]₂,

are the same, as the moiety is typically symmetrical.

It is further preferred that m is independently selected from 1-6, more preferably from 1-4, even more preferably both m are the same and selected from 1-6, most preferably both m are the same and selected from 1-4.

According to the embodiment, R⁸ is as defined above, but preferably the R⁸ radical is selected from linear alkylene groups and linear alkenylene groups, in particular from linear C6-C24 alkylene groups such as hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or linear C6-C24 alkenylene groups such as hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene, wherein the groups are most preferably bonded to the adjacent C(O) group by a terminal C-atom.

More preferably, R⁸ is derived from C7-C25 fatty acids bearing one hydroxyl group as substituent, even more preferably R⁸ is derived from ricinoleic acid, lesquerolic acid, 10-hydroxy octadecanoic acid, 12-hydroxy octadecanoic acid, 14-hydroxy tetradecanoic acid, 10-hydroxy stearic acid, 12-hydroxy stearic acid.

Most preferably R⁸ is derived from ricinoleic acid.

It is generally preferred in this embodiment that all R⁸ groups of a moiety of the general formula

R^{1*}[(—O—C(O)—R⁸)ₘ—O—C(O)—]₂,

are the same.

In a specifically preferred embodiment of the invention, in at least one moiety of the general formula

R^{1*}[(—O—C(O)—R⁸)ₘ—O—C(O)—]₂,

R^{1*} is selected from methylene, ethylene, 1,3-propylene, 1,4-butylene, 1,6-hexylene, 1,2-propylene, 1,3-butylene,

R⁸ is derived from C8-C24 monocarboxy-monohydroxy carboxylic acids, in particular from ricinoleic acid, 12-hydroxy stearic acid, lesquerolic acid, 11-hydroxy-undecanoic acid, and m is independently selected from 1 to 6.

The present application is further directed at another aspect of the invention, which is a process for the manufacture of the organic ammonium salt according to the invention as defined in any of the embodiments according to the invention as described herein.

The process for the manufacture of the organic ammonium salt according to the invention comprises:
(i) The reaction of an organic amine corresponding to the organic ammonium-group-comprising cation with a carboxylic acid (—COOH) corresponding to the carboxylate anion (COO⁻) group-comprising anion selected from the group consisting of the formulas (V), (VII) and (X); or
(ii) The reaction of an inorganic anion-containing salt of the organic ammonium-group-comprising cation with a metal salt of the carboxylic acid (—COOH) corresponding to the carboxylate anion (COO⁻) group-comprising anion selected from the group consisting of the formulas (V), (VII) and (X); or
(iii) The reaction of a hydroxy salt of the organic ammonium-group-comprising cation with the carboxylic acid corresponding to the carboxylate anion (COO⁻) group-comprising anion selected from the group consisting of the formulas (V), (VII) and (X).

While in alternative (i) the ammonium cation and the carboxylate anion are formed by transfer of a proton from the carboxylic acid to the organic amine and accordingly no further products are formed in the reaction, in the process of alternative (ii) one equivalent of a salt consisting of metal cations and inorganic anions, such as alkaline metal or earth alkaline metal halides, in particular NaCl, KCl, NaBr, NaCl, NaI, and KI is formed, and in alternative (iii) one equivalent of water is formed when obtaining the organic ammonium salt according to the invention.

Accordingly, in organic ammonium salts obtained by the processes of alternatives (ii) and (iii), all ammonium groups may be quaternary ammonium moieties, while the ammonium salts according to the invention obtained by the process of alternative (I) comprise at least one tertiary, secondary or primary ammonium moiety.

In alternative (ii) according to the embodiment, ammonium salts having inorganic anions are applied for the formation of the organic ammonium salts according to the invention. Therein, the term “inorganic anion” refers to
halide anions, in particular fluoride, chloride, bromide and iodide anions, hydroxyl anions, hydrosulfide anions,
anions formed as conjugate bases of inorganic oxoacids, for instance chlorate, chlorite, iodate, nitrate, nitrite, perchlorate, phosphate, sulfate or sulfite anions, in particular phosphate and sulfate anions,
   inorganic oxoacid-based ester anions, such as sulfuric acid ester anions, for example methylsulfate, and phosphoric ester acid ester anions, such as monomethylphosphate and dimethylphosphate.

In a preferred embodiment according to the invention, the process for the manufacture of the organic ammonium salt comprises the anion exchange reaction step (ii), and alkali metal salts or alkaline earth metal salts of the carboxylic acid (—COOH) corresponding to the carboxylate anion (COO⁻) group-comprising anion selected from the group consisting of the formulas (V), (VII) and (X) are applied.

Therein, Li, Na, K, Mg and Ca salts are preferred, most preferably the carboxylic acid salts are Na or K salts.

In another preferred embodiment according to the invention, the manufacture of the organic ammonium salt according to the invention comprises (ii) the reaction of an inorganic anion-containing salt of the organic ammonium-group-comprising cation with a metal salt of the carboxylic acid (—COOH) corresponding to the carboxylate anion (COO⁻) group-comprising anion selected from the group consisting of the formulas (V), (VII) and (X), wherein

a Na or K carboxylate comprising the carboxylate anion (COO⁻) group-comprising anion selected from the group consisting of the formulas (V), (VII) and (X) is contacted with an inorganic anion-containing salt of the organic ammonium-group-comprising cation selected from chloride, bromide or methosulphate anion-containing primary, secondary, tertiary, quaternary amine salts.

Usually, the inorganic anion of the organic ammonium salt stems from the formation of the salt by a substitution reaction of an amine and an organic compound bearing a leaving group, such as an iodo, bromo, chloro, mesylate or tosylate leaving group. In this embodiment, the use of inorganic anion-containing salts comprising chloride, bromide or methosulfate anions is preferred, most preferably amine salts having chloride or bromide anions are applied.

In a further preferred embodiment according to the invention, in the process according to the invention the anion exchange reaction (ii) yielding the target salt compounds according to any of the embodiments according to the invention is carried out in a separate reaction step prior to any contact with other ingredients of the final cosmetic formulations.

Herein, according to the invention the term “other ingredients of the final cosmetic formulations” includes all constituents of such cosmetic formulation composition except for water and the salt formed as a by-product in step (ii), i.e. according to this embodiment the ammonium salt compound or compounds may also be added to a cosmetic formulation as an aqueous solution optionally also containing the salts or salts formed as by-products in the reaction (ii).

In still another preferred embodiment according to the invention, in the process according to the invention the alkali salts of the carboxylic acid (—COOH) corresponding to the carboxylate anion (COO⁻) group-comprising anion selected from the group consisting of the formulas (V), (VII) and (X) are added to partial cosmetic formulations or the complete cosmetic formulations containing the inorganic counter ions-containing primary, secondary, tertiary, quaternary amine salts.

Preferably, the alkali salts of the carboxylic acid are selected from sodium and potassium salts.

According to this embodiment, the organic ammonium salts according to the invention are formed in the partial or complete cosmetic formulation, wherein as a side product a salt of an alkali metal cation and an inorganic anion is formed.

In another preferred embodiment according to the invention, the process for the manufacture of the organic ammonium salt according to any of the embodiments of the invention comprises the anion exchange reaction step (iii) as defined above, and therein the anion exchange reaction step (iii) of contacting the carboxylic acid corresponding to the carboxylate anion (COO−) group-comprising anion selected from the group consisting of the formulas (V), (VII) and (X) with a primary, secondary, tertiary and amines quaternary ammonium ion having an OH− counter ion is carried out in a separate reaction step prior to any contact with other ingredients of the final cosmetic formulations.

Herein, according to this embodiment the term “other ingredients of the final cosmetic formulations” includes all constituents of such cosmetic formulation composition except for water i.e. according to this embodiment the ammonium salt compound or compounds may also be added to a cosmetic formulation as an aqueous solution.

In still another preferred embodiment according to the invention, the process for the manufacture of the organic ammonium salt according to any of the embodiments of the invention comprises the anion exchange reaction step (iii) as defined above, and therein the carboxylic acid corresponding to the carboxylate anion (COO⁻) group-comprising anion selected from the group consisting of the formulas (V), (VII) and (X) is added to partial cosmetic formulations or the complete cosmetic formulations containing primary, secondary, tertiary, quaternary amine salts having an OH⁻ counter ion for contacting.

According to this embodiment, the organic ammonium salts according to the invention are formed in the partial or complete cosmetic formulation, wherein one equivalent of water is formed as a side product.

Another aspect of the invention is the use of the compounds according to the invention as defined in any of the previous embodiments in cosmetic formulations for skin care and conditioners and shampoos for hair care.

In a preferred embodiment according to the invention, the salt compounds of any of the previous embodiments are used in cosmetic formulations for skin care and conditioners and shampoos for hair care, in particular conditioners and shampoos, in polishing agents for treating and coating hard surfaces, in formulations for drying automobiles and other hard surfaces, for example following automatic washing, for finishing textiles and textile fibers, as separate softeners for use after textiles have been washed with nonionic or anionic/nonionic detergent formulations, as softeners in formulations for washing textiles that are based upon nonionic or anionic/nonionic surfactants, and as means for preventing or removing wrinkles in textiles.

As shown in the Examples, the organic ammonium salts according to the invention display a distinct conditioning effect on fibers and may such be used in a beneficial manner in the applications according to this embodiment. When formulations comprising the organic ammonium salts according to the invention are applied to hard surfaces as mentioned above, a hydrophobic coating is formed.

In a further preferred embodiment of the invention, the salt compounds of any of the embodiments according to the invention are used in cosmetic compositions for the treatment of fibers, preferred amino acid based fibers, more preferred human hair, in particular being useful for hair color retention, for hair shine enhancement, for hair color enhancement, for hair color protection, for hair conditioning, for hair smoothening or softening, for improving manageability of the hair, and in particular for improving the combability of the hair.

The organic ammonium salt compounds according to the invention can be used in such a manner as defined in this embodiment due to their distinct conditioning properties.

In another aspect, the invention is directed at compositions containing the salt compounds as defined in any of the embodiments according to the invention for the treatment of hair.

In a preferred embodiment according to the invention, the compositions containing the salt compounds of any of the embodiments according to the invention for the treatment of hair are selected from the group consisting of hair shampoo compositions, hair conditioning compositions, hair coloration or dyeing compositions, hair combability improving compositions, hair rinse-off and leave-on compositions.

In yet a further aspect, the invention is also directed at the use of the products as obtained in the process as defined in the above-described embodiments of the invention.

In a preferred embodiment according to the invention, the products obtained from the process as defined in the above-described embodiments of the invention are used in cosmetic formulations for skin care and conditioners and shampoos for hair care, in particular conditioners and shampoos, in polishing agents for treating and coating hard surfaces, in formulations for drying automobiles and other hard surfaces, for example following automatic washing, for finishing textiles and textile fibers, as separate softeners for use after textiles have been washed with nonionic or anionic/nonionic detergent formulations, as softeners in formulations for washing textiles that are based upon nonionic or anionic/nonionic surfactants, and as means for preventing or removing wrinkles in textiles.

In a further preferred embodiment according to the invention, the products obtained from the process as defined in the above-described embodiments of the invention are used in cosmetic compositions for the treatment of fibers, preferred amino acid based fibers, more preferred human hair, in particular being useful, for hair color retention, for hair shine enhancement, for hair color enhancement, for hair color protection, for hair conditioning, for hair smoothening or softening, for improving manageability of the hair, an in particular for improving the combability of the hair.

Finally, in another aspect, the invention is directed at compositions containing the salt compounds as obtained from the process as defined in the above-described embodiments of the invention for the treatment of hair.

In a preferred embodiment according to the invention, the compositions containing the salt compounds as obtained in the processes as defined in the above-described embodiments of the invention for the treatment of hair are selected from the group consisting of hair shampoo compositions, hair conditioning compositions, hair coloration or dyeing compositions, hair combability improving compositions, hair rinse-off and leave-on compositions.

### EXAMPLES

(The percentages refer to weight-% unless otherwise indicated).

As used herein, the term “castor oil” generally refers to ricinoleic acid triglyceride).

### Remarks on the Nomenclature Used Herein for Estolide Moities and Estolide Compounds

In the nomenclature for denoting the structure of the estolide groups as used in the following examples, which refers to the compounds from which the estolide moieties are at least formally obtained by esterification, the carboxylic acids from which the estolide moieties are at least formally derived are given in a sequential manner in parentheses. In case there are several subunits derived from the same acid in a row present in the estolide moiety and these are indicated in parentheses, wherein a subscript integer indicates the number of repeating units, the carboxylic acids are given in brackets.

It is noted that the specific carboxylic acids given in parentheses or brackets are not combined in a random structure, but they have exactly the sequence of hydroxyl-carboxylic acid-derived residues and carboxylic acid-derived residues, respectively, as indicated in the term used. Therein, the last carboxylic acid given in the term in parentheses or brackets, respectively, is the terminal carboxylic acid of the estolide moiety. Going from the beginning of the term in parentheses or brackets to the end of the term, the order of carboxylic acid residues linked by ester groups is displayed in the correct order and number of residues contained.

For example, the term “(12-hydroxy stearic acid-ricinoleic acid-oleic acid)” refers to an estolide moiety in which formally 12-hydroxy stearic acid molecule is linked via its OH group to the carboxylic acid group of a ricinoleic acid molecule by forming an ester group. The hydroxyl group of the said ricinoleic acid group is linked to an oleic acid molecule by forming an ester group with the carboxylic acid group of the oleic acid molecule. The oleic acid is in this example considered to be the terminal group of this specific estolide moiety, as, if the estolide moiety is a substituent of a higher-level structure (i.e. a more complex molecule), in general the estolide moiety is linked to the overall structure via linkage to the carboxylic acid group of the first mentioned residue of the term used for the estolide moiety. In this case, this is the first mentioned 12-hydroxy stearic acid residue, and the oleic acid residue is the terminal group of the estolide moiety.

Accordingly, in case the term used refers to a carboxylic acid chloride of an estolide structure, the acyl chloride group is necessarily formed from the carboxylic acid group of the first-mentioned carboxylic acid residue in parentheses, i.e. the most remote one from the terminal group.

In case terms as “dimer” or “trimer” and so on are used, this refers to the number of carboxylic acid-derived subunits of the estolide moieties.

In the same manner, the term “[(ricinoleic acid)₂-oleic acid] estolide” refers to an estolide moiety or compound in which formally a ricinoleic acid molecule or residue is linked via its OH group to the carboxylic acid group of a further ricinoleic acid molecule by forming an ester group. The hydroxyl group of the latter ricinoleic acid group mentioned is linked to an oleic acid molecule by forming an ester group with the carboxylic acid group of the oleic acid molecule. The oleic acid is considered to be the terminal group of this specific estolide moiety, as, if the estolide moiety is a substituent of a higher-level structure (i.e. a more complex molecule), the estolide moiety is linked to the overall structure via linkage to the carboxylic acid group of the first. mentioned ricinoleic acid residue, and the oleic acid residue is the terminal group of the estolide moiety.

In the case estolide moieties are linked by a linking group via ester or amide groups, such as by the succinic acid derived residue in [(ricinoleic acid)₆-succinic acid-(ricinoleic acid)₆], linked to two ricinoleic acid estolide groups by an ester group on each side, this is indicated by incorporation of the name of the parent compound into the term applied to the overall estolide structure. Thus, a comprehensive term indicating the sequence of carboxylic acid residues is provided.

It is further noted that the exact structure of the estolides is primarily clarified by the structural formulas, which are thoroughly provided for the example compounds, and that the structures of the example compounds can also be clearly derived by the skilled artisan from the detailed experimental procedures provided. In particular, for reasons of simplification, the formulae do not always reflect the stereochemically correct structures.

### EXAMPLES

### Synthesis Example 1

### Synthesis of a (Ricinoleic Acid-Oleic Acid) Estolide Dimer

In a 1000 ml four-necked bottle, equipped with refluxing condenser, thermometer and magnetic stirrer, dropping funnel and gas outlet tube 225 g (0.75 mol) ricinoleic acid were placed at room temperature under a nitrogen atmosphere. Upon stirring, 226.85 g (0.75 mol) oleic acid chloride were added slowly during 1.5 h. The temperature increased from 22 to 32° C. The temperature increase was accompanied by the generation of gas bubbles indicating the formation of HCl. The temperature was maintained at 32° C. for further 2 h, afterwards it was increased to 50° C. and was maintained there for 1 h. Volatiles were removed under reduced pressure (40° C./2 h/20 mmHg). The conversion of the OH groups was determined by means of ¹H NMR spectroscopy. The conversion of the OH groups was 100%.

A brownish, transparent oil essentially having the following structure was obtained:

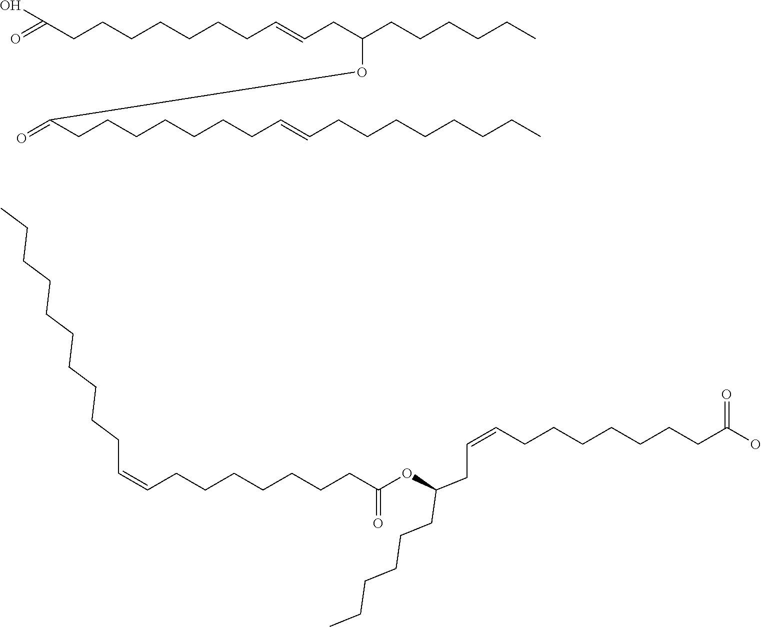

The corresponding estolide-based carboxylate anion is covered by formula (X):

R⁷(—C(O)—X—R⁸—COO⁻)_{q}

for X=O and q=1, and

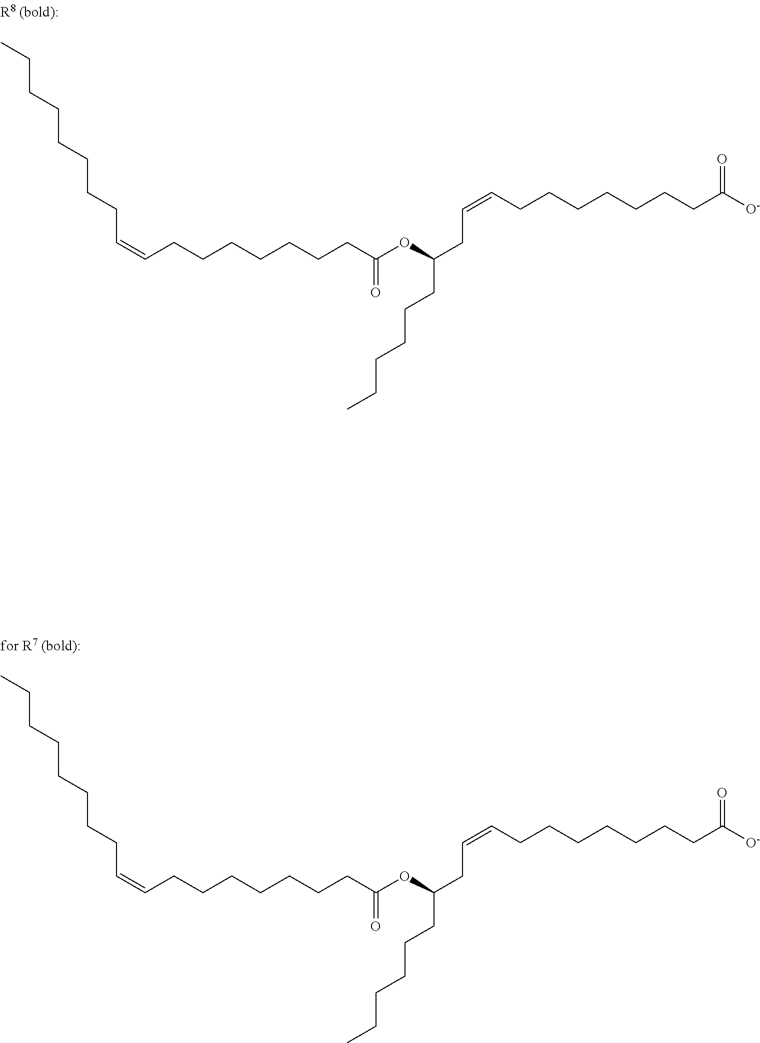 [SMILES: CCCCCCCC/C=C\CCCCCCCC(=O)O[C@@H](C/C=C\CCCCCCCC(=O)[O-])CCCCCC ; CCCCCCCC/C=C\CCCCCCCC(=O)O[C@@H](C/C=C\CCCCCCCC(=O)[O-])CCCCCC]

### Synthesis Example 2

### Synthesis of a [(ricinoleic acid)₂-oleic acid] Estolide Trimer

In a 250 ml four-necked bottle, equipped with refluxing condenser, thermometer and magnetic stirrer, dropping funnel and gas outlet tube 34.87 g (0.293 mol) SOCl₂ were placed at room temperature under a nitrogen atmosphere. Upon stirring, 110 g (0.195 mol) of the estolide dimer of synthesis example 1 were added slowly during 1 h. After the end of the addition the temperature was increased to 80° C. The temperature was maintained at 80° C. for 1 h. Volatiles were removed under reduced pressure (80° C./2 h/20 mmHg). Nitrogen was used to break the vacuum and 57.75 g (0.195 mol) ricinoleic acid were added to the carboxylic acid chloride intermediate at 80° C. over 45 minutes. The temperature was maintained for 2 h, Volatiles were removed under reduced pressure (40° C./2 h/20 mmHg). The conversion of the OH groups was determined by means of ¹H NMR spectroscopy. The conversion of the OH groups was 100%. A brownish, transparent oil essentially having the following structure was obtained:

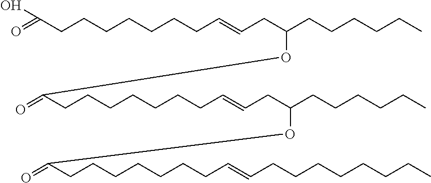 [SMILES: CCCCCCCC/C=C/CCCCCCCC(=O)OC(C/C=C/CCCCCCCC(=O)OC(C/C=C/CCCCCCCC(=O)O)CCCCCC)CCCCCC]

### Example 2a

### Synthesis of a [(ricinoleic acid)₂-stearic acid] Estolide Trimer

Two 250 ml three-necked bottles A and B, equipped with refluxing condenser, thermometer and magnetic stirrer, dropping funnel and gas outlet tube were flushed with nitrogen.

Bottle A was used to react fatty acid chlorides with ricinoleic acid yielding a chain extended fatty ester acid. Subsequent addition of SOCl₂ yielded the corresponding fatty ester acid chloride.

Bottle B was used to react the formed fatty ester acid chloride with ricinoleic acid yielding a chain extended fatty ester acid. Subsequent addition of SOCl₂ yielded the corresponding fatty ester acid chloride. This fatty acid chloride was transferred back to bottle A and reacted with fresh ricinoleic acid. The above described cycle may be repeated until the hexamer estolide [(ricinoleic acid)₅-stearic acid] is prepared.

### General Procedure for the Synthesis of Chain Extended Fatty Ester Acids:

The calculated amount of ricinoleic acid was placed in a bottle. An equimolar amount of fatty ester acid chloride was added slowly at room temperature. In order to complete the reaction, the temperature was increased to 80° C. for 3 h. The complete conversion of the OH groups was determined by means of ¹H NMR spectroscopy.

### General Procedure for the Synthesis of Fatty Ester Acid Chlorides:

The calculated amount fatty ester acid was placed in a bottle. SOCl₂ (threefold molar excess) was added slowly at room temperature. Afterwards, the mixture was heated to 80° C. The temperature was maintained for 3 h. Afterwards, the excess of SOCl2 was removed under reduced pressure (80° C./2 h/20 mmHg). The complete conversion of the C(O)OH groups to C(O)Cl groups was determined by means of ¹H NMR spectroscopy.

The following table summarizes the materials and the quantities used.

| | | | fatty acid and | | |
|---|---|---|---|---|---|
| | | fatty ester | amount of | SOCl₂ | |
| | fatty ester | derivative | fatty | amount | |
| bottle | derivative | amount [g] | acid [g] | [g] | target product |
| A | stearic acid | 55.83 | ricinoleic | | (rici-stearic acid) |
| | chloride | | acid | | |
| | | | 55.00 | | |
| A | (rici-stearic | 104.12 | | 32.9 | (rici-stearic acid) |
| | acid) | | | | chloride |
| B | (rici-stearic | 103.85 | ricinoleic | | [(rici)₂-stearic |
| | acid) | | acid | | acid] |
| | chloride | | 53.13 | | |

Note:
The term “rici” replaces the term “ricinoleic acid” in denoting a ricinoleyl radical.

The formula of [(rici)₂-stearic acid] is as follows:

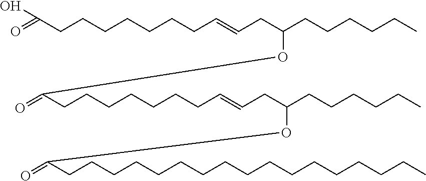 [SMILES: CCCCCCCCCCCCCCCCCC(=O)OC(C/C=C/CCCCCCCC(=O)OC(C/C=C/CCCCCCCC(=O)O)CCCCCC)CCCCCC]

### Example 2b

### Synthesis of a (ricinoleic acid-12-hydroxy stearic acid-oleic acid) Estolide Trimer

The procedure outlined for synthesis example 2a was repeated.

The following table summarizes the materials and the quantities used.

| | | | fatty acid | | |
|---|---|---|---|---|---|
| | | | and | | |
| | | fatty ester | amount of | SOCl₂ | |
| | fatty ester | derivative | fatty | amount | |
| bottle | derivative | amount [g] | acid [g] | [g] | target product |
| A | oleic acid | 55.37 | 12-hydroxy | | (12-hydroxy |
| | chloride | | stearic acid | | stea- |
| | | | 55.3 | | oleic acid) |
| A | (12-hydroxy | 103.27 | | 39.8 | (12-hydroxy |
| | stea- | | | | stea- |
| | oleic acid) | | | | oleic acid) |
| | | | | | chloride |
| B | (12-hydroxy | 104.8 | ricinoleic | | (rici- |
| | stea- | | acid | | 12 hydroxy |
| | oleic acid) | | 53.61 | | stea- |
| | chloride | | | | oleyl acid) |

Note:
The term “rici” replaces the term “ricinoleic acid” in denoting a ricinoleyl radical, the term “12-hydroxy-stea” replaces the term “12-hydroxy stearic acid” in denoting a 12-hydroxyl stearyl radical.

The formula of rici-(12 hydroxy stea)-oleic acid is as follows:

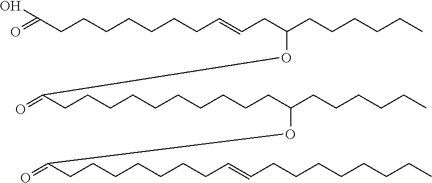 [SMILES: CCCCCCCC/C=C/CCCCCCCC(=O)OC(CCCCCC)CCCCCCCCCCC(=O)OC(C/C=C/CCCCCCCC(=O)O)CCCCCC]

### Example 2c

Synthesis of a (12-hydroxy stearic acid-ricinoleic acid-oleic acid) Estolide Trimer

The procedure outlined for synthesis example 2a was repeated.

The following table summarizes the materials and the quantities used.

| | | | fatty acid | | |
|---|---|---|---|---|---|
| | | | and | | |
| | | fatty ester | amount | SOCl₂ | |
| | fatty ester | derivative | of fatty | amount | |
| bottle | derivative | amount [g] | acid [g] | [g] | target product |
| A | oleic acid | 55.00 | ricinoleic | | (rici-oleic acid) |
| | chloride | | acid | | |
| | | | 55.3 | | |
| A | (rici- | 102.91 | | 38.5 | (rici-oleic acid) |
| | oleic acid) | | | | chloride |
| B | (rici- | 100.00 | 12-hydroxy | | (12-hydroxy stea- |
| | oleic acid) | | stearic acid | | rici-oleic acid) |
| | chloride | | 51.68 | | |

Note:
The term “rici” replaces the term “ricinoleic acid” in denoting a ricinoleyl radical, the term “12-hydroxy-stea” replaces the term “12-hydroxy stearic acid” in denoting a 12-hydroxyl stearyl radical.

The formula of (12-hydroxy stea-rici-oleic acid) is as follows:

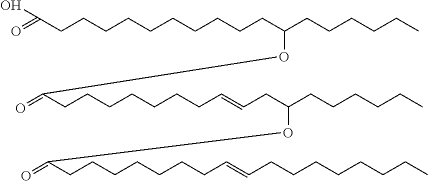 [SMILES: CCCCCCCC/C=C/CCCCCCCC(=O)OC(C/C=C/CCCCCCCC(=O)OC(CCCCCC)CCCCCCCCCCC(=O)O)CCCCCC]

### Synthesis Example 3

### Synthesis of a [(ricinoleic acid)₅-oleic acid] Estolide Hexamer and the Corresponding [(ricinoleic acids-oleic acid] Chloride Hexamer

Two 100 ml three-necked bottles A and B, equipped with refluxing condenser, thermometer and magnetic stirrer, dropping funnel and gas outlet tube were flushed with nitrogen.

Bottle A was used to react fatty acid chlorides with ricinoleic acid, yielding a chain extended fatty ester acid. Subsequent addition of SOCl₂ yielded the corresponding fatty ester acid chloride.

Bottle B was used to react the formed fatty ester acid chloride with ricinoleic acid, yielding a chain extended fatty ester acid. Subsequent addition of SOCl₂ yielded the corresponding fatty ester acid chloride. This fatty acid chloride was transferred back to bottle A and reacted with fresh ricinoleic acid. The above described cycle was repeated until the hexamer estolide [(ricinoleic acid)₅-oleic acid] was prepared.

### General Procedure, for the Synthesis of Chain Extended Fatty Ester Acids:

The calculated amount of ricinoleic acid as placed in a bottle. An equimolar amount of fatty ester acid chloride was added slowly at room temperature. In order to complete the reaction, the temperature was increased to 80° C. for 3 h. The complete conversion of the OH groups of the ricinoleic acid into ester was determined by means of ¹H NMR spectroscopy.

### General Procedure for the Synthesis of Fatty Ester Acid Chlorides:

The calculated amount fatty ester acid was placed in a bottle. SOCl₂ (threefold molar excess) was added slowly at room temperature. Afterwards, the mixture was heated to 80° C. The temperature was maintained for 3 h. Afterwards, the excess of SOCl₂ was removed under reduced pressure (80° C./2 h/20 mmHg). The complete conversion of the C(O)OH groups towards C(O)Cl groups was determined by means of ¹H NMR spectroscopy.

The following table summarizes the materials and the quantities used.

| | | fatty ester | ricinoleic | SOCl₂ | |
|---|---|---|---|---|---|
| | fatty ester | derivative | acid | amount | |
| bottle | derivative | amount [g] | amount [g] | [g] | target product |
| A | oleic chloride | 30.00 | 29.76 | | [(rici)₁-oleic |
| | | | | | acid] |
| A | [(rici)₁-oleic | 59.76 | | 37.89 | [(rici)₁-oleic |
| | acid] | | | | acid] |
| | | | | | chloride |
| B | [(rici)₁-oleic | 46.26 | 23.75 | | [(rici)₂-oleic |
| | acid] | | | | acid] |
| | chloride | | | | |
| B | [(rici)₂-oleic | 70.01 | | 29.63 | [(rici)₂-oleic |
| | acid] | | | | acid] |
| | | | | | chloride |
| A | [(rici)₂-oleic | 52.00 | 18.01 | | [(rici)₃-oleic |
| | acid] | | | | acid] |
| | chloride | | | | |
| A | [(rici)₃-oleic | 70.01 | | 22.23 | [(rici)₄-oleic |
| | acid] | | | | acid] |
| | | | | | chloride |
| | [(rici)₃-oleic | 55.50 | 14.50 | | [(rici)₄-oleic |
| | acid] | | | | acid] |
| | chloride | | | | |
| B | [(rici)₄-oleic | 70.00 | | 17.79 | [(rici)₄-oleic |
| | acid] | | | | acid] |
| | | | | | chloride |
| A | [(rici)₄-oleic | 57.87 | 12.14 | | [(rici)₅-oleic |
| | acid] | | | | acid] |
| | chloride | | | | |
| A | [(rici)₅-oleic | 50 | | 10.60 | [(rici)₅-oleic |
| | acid | | | | acid] |
| | | | | | chloride |

Note:
The term “rici” replaces the term “ricinoleic acid” in denoting a ricinoleyl radical, the term “12-hydroxy-stea” replaces the term “12-hydroxy stearic acid” in denoting a 12-hydroxyl stearyl radical.

A brownish, transparent oil essentially having the following structure [(rici)₅-oleic acid] was obtained:

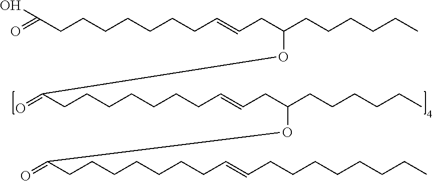

The corresponding [(rici)₅-oleic acid] chloride has the structure:

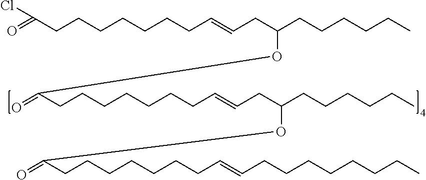

### Synthesis Example 4

### Synthesis of a Branched bis-[(ricinoleic acid)₂-oleic acid] Estolide Based on bis 2,2-di-hydroxymethyl propionic acid

In a 100 ml three-necked bottle, equipped with refluxing condenser, thermometer and magnetic stirrer, dropping funnel and gas outlet tube 48.88 g (0.0567 mol) of the [(ricinoleic acid)₂-oleic acid] chloride of synthesis example 3 were mixed with 3.80 g (0.0284 mol) 2,2-di-hydroxymethyl propionic acid. The mixture was heated to 100° C. for 8 h. Volatiles were removed under reduced pressure (80° C./1 h/20 mmHg). The complete conversion of the OH groups of 2,2-hydroxymethyl propionic acid was determined by means of ¹H NMR spectroscopy.

A brownish, transparent oil essentially having the following structure was obtained:

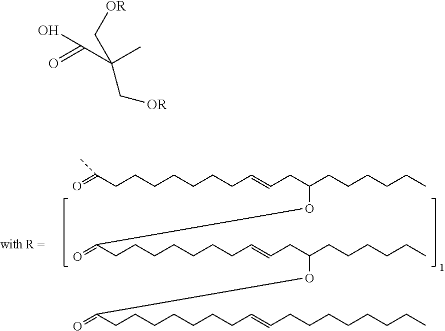

### Example 4a

### Synthesis of a Dendritic bis-[(ricinoleic acid)₂-oleic acid] Estolide

In a 100 ml three-necked bottle, equipped with refluxing condenser, thermometer and magnetic stirrer, dropping funnel and gas outlet tube 64.57 g (0.03617 mol) of the branched bis-[(ricinoleic acid)₂-oleic acid] estolide based on 2,2-di-hydroxymethyl propionic acid of synthesis example 4 were heated 80° C. 8.61 g (0.0723 mol) SOCl₂ were added within 10 minutes. The reaction is maintained for 4 h. Volatiles were removed under reduced pressure (80° C./1 h/20 mmHg). The complete conversion of the C(O)OH groups to C(O)Cl groups was determined by means of ¹H NMR spectroscopy. 2.42 g (0.01808 mol) 2,2-di-hydroxymethyl propionic acid were added at 80° C. and the reaction was maintained for additional 5 h. Volatiles were removed under reduced pressure (80° C./0.5 h/20 mmHg). The complete conversion of the terminal OH groups of the bis-2,2-di-hydroxymethyl propionic acid was determined by means of ¹H NMR spectroscopy.

A viscous brownish, transparent oil essentially having the following dendrimeric structure was obtained:

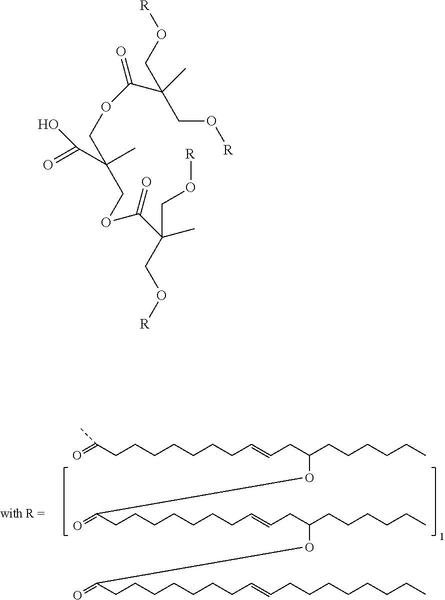

### Example 4b

### Synthesis of a Branched bis-[(ricinoleic acid)₂-stearic acid] Estolide Based on 2,2-di-hydroxymethyl propionic acid

In a 250 ml three-necked bottle, equipped with refluxing condenser, thermometer and magnetic stirrer, dropping funnel and gas outlet tube 95.06 g (0.1124 mol) of the [(ricinoleic acid)₂-stearic acid] estolide of synthesis example 2a were heated to 80° C. 33.8 g (0.28 mol) SOCl₂ were added within 10 minutes. The reaction was maintained for 4 h. Volatiles are removed under reduced pressure (80° C./1 h/20 mmHg). The complete conversion of the C(O)OH groups towards C(O)Cl groups was determined by means of ¹H NMR spectroscopy.

In a 250 ml three-necked bottle, equipped with refluxing condenser, thermometer and mechanical stirrer, dropping funnel and gas outlet tube 92.88 g (0.1075 mol) of the (ricinoleic acid)₂-stearic acid chloride intermediate and 7.22 g (0.0538 mol) 2,2-di-hydroxymethyl propionic acid were mixed at 80° C. and the reaction was maintained for additional 5 h. Volatiles were removed under reduced pressure (80° C./0.5 h/20 mmHg). The complete conversion of the OH groups of the 2,2-di-hydroxymethyl propionic acid was determined by means of ¹H NMR spectroscopy.

A viscous brownish, transparent oil essentially having the following structure was obtained:

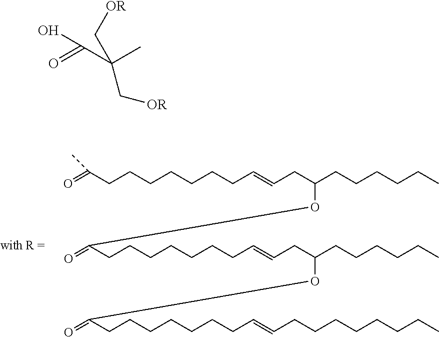

### Example 4c

### Synthesis of a Branched bis-(ricinoleic acid-12-hydroxy stearic acid-oleic acid) Estolide Based on 2,2-di-hydroxymethyl propionic acid

In a 250 ml three-necked bottle, equipped with refluxing condenser, thermometer and magnetic stirrer, dropping funnel and gas outlet tube 102.95 g (0.1218 mol) of the (ricinoleic acid-12 hydroxy stearic acid-oleic acid) estolide of synthesis example 2b were heated to 80° C. 42.8 g (0.36 mol) SOCl₂ were added within 10 minutes. The reaction was maintained for 4 h. Volatiles were removed under reduced pressure (80° C./1 h/20 mmHg). The complete conversion of the C(O)OH groups to C(O)Cl groups was determined by means of ¹H NMR spectroscopy,

In a 250 ml three-necked bottle, equipped with refluxing condenser, thermometer and mechanical stirrer, dropping funnel and gas outlet tube 104.22 g (0.1206 mol) of the bis-(ricinoleic acid-12 hydroxy stearic acid-oleic acid) chloride intermediate and 8.09 g (0.0603 mol) 2,2-di-hydroxymethyl propionic acid were mixed at 80° C. and the reaction was maintained for additional 5 h. Volatiles were removed under reduced pressure (80° C./0.5 h/20 mmHg). The complete conversion of the OH groups from 2,2-di-hydroxymethyl propionic acid was determined by means of ¹H NMR spectroscopy.

A viscous brownish, transparent oil essentially having the following structure was obtained:

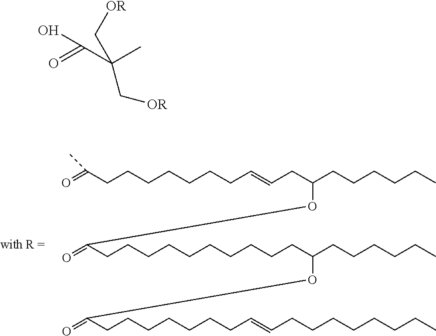

### Example 4d

### Synthesis of a Branched (12-hydroxy stearic acid-ricinoleic acid-oleic acid) Estolide Based on 2,2-di-hydroxymethyl propionic acid

In a 250 ml three-necked bottle, equipped with refluxing condenser, thermometer and magnetic stirrer, dropping funnel and gas outlet tube 98.05 g (0.116 mol) of the (12-hydroxy stearic acid-ricinoleic acid-oleic acid) estolide of synthesis example 2c were heated to 80° C. 30.08 g (0.25 mol) SOCl₂ were added within 10 minutes. The reaction was maintained for 4 h. Volatiles were removed under reduced pressure (80° C./1 h/20 mmHg). The complete conversion of the C(O)OH groups to C(O)Cl groups was determined by means of ¹H NMR spectroscopy.

In a 250 ml three-necked bottle, equipped with refluxing condenser, thermometer and mechanical stirrer, dropping funnel and gas outlet tube 97.4 g (0.1127 mol) of the (12-hydroxy stearic acid-ricinoleic acid-oleic acid) chloride intermediate and 7.56 g (0.0564 mol) 2,2-di-hydroxymethyl propionic acid were mixed at 80° C. and the reaction was maintained for further 5 h. Volatiles were removed under reduced pressure (80° C./0.5 h/20 mmHg). The complete conversion of the OH groups of the hydroxymethyl groups was determined by means of ¹H NMR spectroscopy.

A viscous brownish, transparent oil essentially having the following structure was obtained:

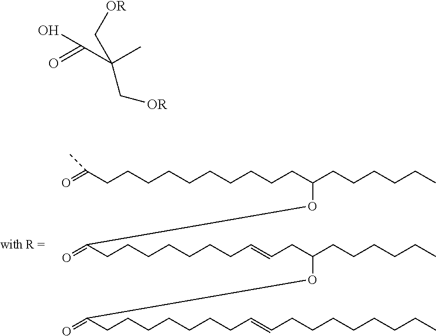

### Synthesis Example 5

### Synthesis of an Alpha Branched bis-[(ricinoleic acid)₅-oleic acid] Estolide Based on 2,2-di-hydroxymethyl propionic acid

In a 100 ml three-necked bottle, equipped with refluxing condenser, thermometer and magnetic stirrer, dropping funnel and gas outlet tube 29.53 g (0.0173 mol) of the [(ricinoleic acid)₅-oleic acid] chloride of synthesis example 3 were mixed with 1.16 g (0.00866 mol) 2,2-di-hydroxymethyl propionic acid. The mixture was heated to 105° C. for 5 h. Volatiles were removed under reduced pressure (80° C./10 min/20 mmHg). The complete conversion of the OH groups of 2,2-di-hydroxymethyl propionic acid was determined by means of ¹H NMR spectroscopy.

A brownish, transparent oil essentially having the following structure was obtained:

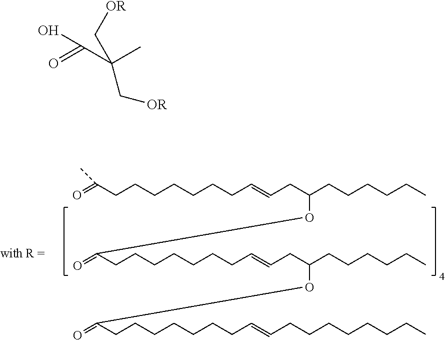

### Synthesis Example 6

### Synthesis of a [(ricinoleic acid)₆-succinic acid-(ricinoleic acid)₆] Diacid Estolide

Two 250 ml three-necked bottles A and B, equipped with refluxing condenser, thermometer and magnetic stirrer, dropping funnel and gas outlet tube were flushed with nitrogen.

Bottle A was used to react the starting material dicarboxylic acid chloride succinyl dichloride or fatty acid chlorides with ricinoleic acid yielding a chain extended fatty ester acid. Subsequent addition of SOCl₂ yielded the corresponding fatty ester acid chloride.

Bottle B was used to react the formed fatty ester acid chloride with ricinoleic acid, yielding a chain extended fatty ester acid. Subsequent addition of SOCl₂ yielded the corresponding fatty ester acid chloride. This fatty acid chloride was transferred back to bottle A and was reacted with fresh ricinoleic acid. The above-described cycle was repeated until the estolide [(ricinoleic acid)₆-succinic acid-(ricinoleic acid)₆] was prepared.

### General Procedure for the Synthesis of Chain Extended Fatty Ester Acids:

The calculated amount of ricinoleic acid was placed in a bottle. An equimolar amount of fatty ester acid chloride was added slowly at room temperature. In order to complete the reaction, the temperature was increased to 80° C. for 3 h. The complete conversion of the OH groups was determined by means of ¹H NMR spectroscopy.

### General Procedure for the Synthesis of Fatty Ester Acid Chlorides:

The calculated amount fatty ester acid was placed in a bottle. SOCl₂ (threefold molar excess) was added slowly at room temperature. Afterwards, the mixture was heated to 80° C. The temperature was maintained for 3 h. Afterwards, the excess of SOCl₂ was removed under reduced pressure (80° C./2 h/20 mmHg). The complete conversion of the C(O)OH groups towards C(O)Cl groups was determined by means of ¹H NMR spectroscopy.

The following table summarizes the materials and the quantities used.

| | | Dicarboxylic | | | |
|---|---|---|---|---|---|
| | | acid | | | |
| | | chloride | | | |
| | | starting | | | |
| | Dicarboxylic acid | material or | | | |
| | chloride starting | fatty ester | ricinoleic | SOCl₂ | |
| | material or fatty | derivative | acid | amount | |
| bottle | ester derivative | amount [g] | amount [g] | [g] | target product |
| A | succinyl dichloride | 30.81 | 118.68 | | [(rici)₁-succ-(rici)₁] |
| | | | | | diacid |
| A | [(rici)₁-succ-(rici)₁] | 135 | | 73.1 | [(rici)₁-succ-(rici)₁] |
| | diacid | | | | dichloride |
| B | [(rici)₁-succ-(rici)₁] | 72.17 | 60.18 | | [(rici)₂-succ-(rici)₂] |
| | dichloride | | | | diacid |
| B | [(rici)₂-succ-(rici)₂] | 125 | | 40.32 | [(rici)₂-succ-(rici)₂] |
| | diacid | | | | dichloride |
| A | [(rici)₂-succ-(rici)₂] | 88.63 | 41.43 | | [(rici)₃-succ-(rici)₃] |
| | dichloride | | | | diacid |
| A | [(rici)₃-succ-(rici)₃] | 125 | | 39.62 | [(rici)₃-succ-(rici)₃] |
| | diacid | | | | dichloride |
| B | [(rici)₃-succ-(rici)₃] | 97.27 | 31.59 | | [(rici)₄-succ-(rici)₄] |
| | dichloride | | | | diacid |
| B | [(rici)₄-succ-(rici)₄] | 125 | | 24.71 | [(rici)₄-succ-(rici)₄] |
| | diacid | | | | dichloride |
| A | [(rici)₄-succ-(rici)₄] | 102.59 | 25.53 | | [(rici)₅-succ-(rici)₅] |
| | dichloride | | | | diacid |
| A | [(rici)₅-succ-(rici)₅] | 125 | | 22.37 | [(rici)₅-succ-(rici)₅] |
| | diacid | | | | dichloride |
| B | [(rici)₅-succ-(rici)₅] | 106.20 | 21.42 | | [(rici)₆-succ-(rici)₆] |
| | dichloride | | | | diacid |

Note:
The term “rici” replaces the term “ricinoleic acid” in denoting a ricinoleyl radical, the term “succ” replaces the term “succinic acid” in denoting a succinyl radical.

A brownish, transparent oil essentially having the following structure [(rici)₆-succinyl-(rici)₆] was obtained:

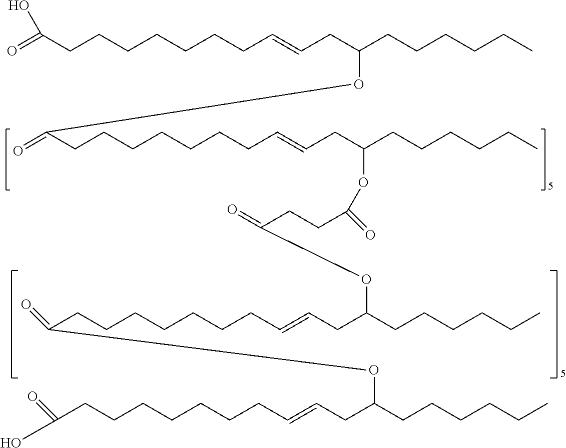

### Synthesis Example 7

### Synthesis of a Castor Oil Based Estolide Bearing a Succinic Acid Mono Ester Moiety

In a 100 ml three-necked bottle, equipped with refluxing condenser, thermometer and magnetic stirrer, dropping funnel and gas outlet tube xg castor oil were mixed with xg succinic anhydride and xg Et3N catalyst. The mixture was heated to 105° C. for 5 h. The complete conversion of the anhydride ring and the formation of ester bonds were determined by means of ¹H NMR spectroscopy.

29.53 g (0.0173 mol) of the ricinoleic acid-oleic acid dimer chloride (intermediate in synthesis example 2) were added. The mixture was heated to 105° C. for 5 h. Volatiles were removed under reduced pressure (80° C./10 min/20 mmHg). The complete conversion of the —C(O)Cl groups and the simultaneous formation of ester bonds were determined by means of ¹H NMR spectroscopy.

A brownish, transparent oil essentially having the following structure was obtained:

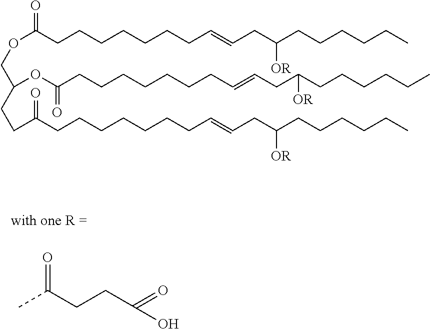

and the remaining two R groups=

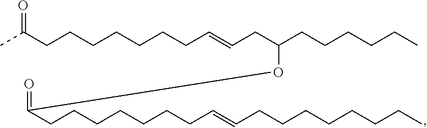

### Synthesis Example 8

### Synthesis of a Hexa-Tertiary Amine Based on Glycerol Diglycidyl Ether

In a 100 ml three-necked bottle, equipped with refluxing condenser, thermometer and mechanic stirrer 0.22 g (0.0016 mol epoxy groups; specific epoxy content 0.00729 mol epoxy groups/1 g) glycerol diglycidyl ether, 0.30 g (0.0016 mol NH groups) (CH₃)₂NCH₂CH₂CH₂NHCH₂CH₂CH₂N(CH₃)₂ and 55 g 2-propanol were mixed at room temperature. The mixture was heated to 80° C. for 8 h. The complete conversion of the epoxy groups was confirmed by means of ¹H NMR spectroscopy.

A brownish liquid containing the following tertiary amine was obtained:

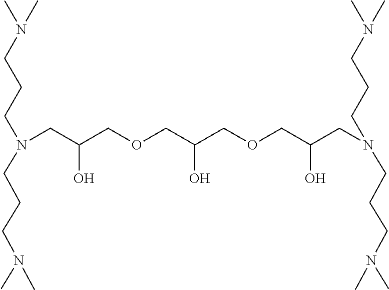

### Synthesis Example 9

### Synthesis of N,N-dimethylaminopropyl oleic acid amide

In a 100 ml three-necked bottle, equipped with refluxing condenser, thermometer and mechanic stirrer 3.4 g (0.033 mol) (CH₃)₂NCH₂CH₂CH₂NH₂ and 13.87 g (0.137 mol) triethylamine are mixed at room temperature. 10 g (0.033 mol) oleic acid chloride are added slowly within 10 minutes. 5.3 g of a 0.2% active aqueous solution of NaOH and afterwards 9.2 g deionized water were added. The aqueous phase is removed after phase separation. In three subsequent washing cycles 11 g deionized water per cycle are added and removed. The solid phase is dissolved in 10 g 2-propanol. Afterwards, volatiles were removed at 38° C./20 mbar.

A brownish oily liquid having the following structure was obtained (structure confirmed by 1H-NMR):

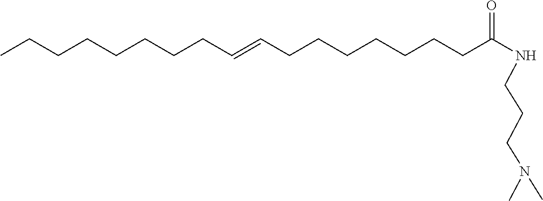 [SMILES: CCCCCCCC/C=C/CCCCCCCC(=O)NCCCN(C)C]

### Example 10

### Poly Fatty Acid Based Salt Compounds

The following table summarizes the salt compounds which were synthesized by combining stoichiometric amounts of the different poly fatty acid based carboxylic acids as described in Synthesis Examples 1 to 7 (—COOH form) or, alternatively, the respective Na carboxylate (—COO⁻ Na⁺ form)), with the respective amino/ammonium compounds.

In order to produce the Na carboxylate form, stoichiometric amounts of aqueous NaOH were added to 2-propanol containing the poly fatty acid based carboxylic acid.

The following amino/ammonium compounds were used:
oleylamine
lysine
N,N-dimethylaminopropyl oleic acid amide according to Synthesis Example 9
Polyquaternium 16
tetrabutyl ammonium bromide

| | | poly fatty acid based | |
|---|---|---|---|
| salt | | carboxylic acid or | |
| compound | | Na carboxylate from | solvent |
| example | amino/ammonium structure | Synthesis Example | composition |
| 10.a | oleylamine | 3 (—COOH form) | 2-propanol |
| 10.b | oleylamine | 4a (—COOH form) | 2-propanol |
| 10.c | oleylamine | 6 (—COOH form) | 2-propanol |
| 10.d | lysine | 2 (—COOH form) | 2-propanol |
| 10.e | lysine | 2a (—COOH form) | 2-propanol |
| 10.f | lysine | 4a (—COOH form) | 2-propanol |
| 10.g | N,N-dimethylaminopropyl oleic | 2a (—COOH form) | 2-propanol |
| | acid amide (example 9) | | |
| 10.h | N,N-dimethylaminopropyl oleic | 2b (—COOH form) | 2-propanol |
| | acid amide (example 9) | | |
| 10.i | N,N-dimethylaminopropyl oleic | 2c (—COOH form) | 2-propanol |
| | acid amide (example 9) | | |
| 10.j | N,N-dimethylaminopropyl oleic | 7 (—COOH form) | 2-propanol |
| | acid amide (example 9) | | |
| 10.k | Polyquaternium-16 | 3 (—COO⁻ Na⁺ form) | 2-propanol/H₂O |
| 10.l | tetrabutyl ammonium bromide | 2 (—COOH form) | 2-propanol/H₂O |
| 10.m | tetrabutyl ammonium bromide | 2 (—COO⁻ Na⁺ form) | 2-propanol/H₂O |

### Application Tests

### Combing Force Measurements

Combing force measurements were carried out in order to quantify the effect of the compounds according to the invention. A Miniature Tensile Tester 175 (Dia-Stron Limited) was used.

Heavily bleached Caucasian hair (Kerling International) were selected for these measurements:

| Hair type | Hair finishing method |
|---|---|
| Euro-Hair, bleached heavily, 20 cm long | 1 |
| “damaged human hair” | |

### Hair Finishing Method 1 (Damaged Human Hair)

The weight of the portion of the hair tresses to be finished is determined and the calculated total amount on active substance (based on the target mg active/1 g buffalo hair) dissolved in 2-propanol or 2-propanol-H₂O mixtures. The amount on 2-propanol used was calculated by the following formula:

m₂₋ₚᵣₒₚₐₙₒₗ (g)=0.64×m_{hair-finished}

The 2-propanol or 2-propanol/water solutions are evenly distributed over the hair tresses. The tresses are air dried for 2 h and further processed as outlined in the general protocol.

### General Protocol for the Pretreatment and Handling of Hair Tresses

Individual hair tresses (2.5 cm) were cut off from the respective stock tress and equilibrated in a humidity chamber at 50% relative humidity (rel. hum.) for 12 h. Afterwards, the dry tear off force and the wet average force (tresses rinsed with 38° C. tap water for 30 seconds) were determined for the untreated tresses (baseline measurements). Three strokes were carried out. The force data of the third stroke were used for the calculations.

The tresses were air dried and equilibrated in the climate chamber for additional 15 h. Afterwards, they were finished with the 2-propanol solutions as outlined for the hair finishing methods 1 and 2, air dried for two hours and equilibrated in the climate chamber for additional 15 h. Finally, the dry tear off force and the wet average force (tresses rinsed with 38° C. tap water for 30 seconds) were determined for the finished tresses (measurements finished hair). Three strokes were carried out. The force data of the third stroke were used for the calculations.

The ratio between the required combing force before finishing (baseline measurements) and the combing force after finishing (measurements finished hair) describes the effectiveness of a conditioning agent.

The following formula was used to calculate the relative combing force reduction:

Force reduction (%)=(Force_{baseline}−Force_{finished})×100/Force_{baseline}

### Results Combing Force Measurements

### Damaged Human Hair

| | | poly fatty | | | |
|---|---|---|---|---|---|
| | | acid based | | | |
| | | carboxylic | concentration | | wet |
| salt | | acid or Na | active | dry tear | average |
| compound | amino/ammonium | carboxylate | (mg active/ | off force | force |
| example | structure | from example | 1 g hair) | (reduction %) | (reduction %) |
| 10.a | oleylamine | 3 (—COOH | 5 | 98.2 | 83.8 |
| | | form) | | | |
| 10.b | oleylamine | 4a (—COOH | 5 | 58.6 | 45.3 |
| | | form) | | | |
| 10.c | oleyiamine | 6 (—COOH | 5 | 94.4 | 77.6 |
| | | form) | | | |
| 10.d | lysine | 2 (—COOH | 5 | 82.5 | 90.0 |
| | | form) | | | |
| 10.e | lysine | 2a (—COOH | 5 | 64.8 | 67.0 |
| | | form) | | | |
| 10.f | lysine | 4a (—COOH | 5 | 96.4 | 79.0 |
| | | form) | | | |
| 10.g | N,N- | 2a (—COOH | 5 | 84.9 | 94.0 |
| | dimethylaminopropyl | form) | | | |
| | oleic acid amide | | | | |
| | (example 9) | | | | |
| 10.h | N,N- | 2b (—COOH | 5 | 22.4 | 46.0 |
| | dimethylaminopropyl | form) | | | |
| | oleic acid amide | | | | |
| | (example 9) | | | | |
| 10.i | N,N- | 2c (—COOH | 5 | 91.9 | 68.5 |
| | dimethylaminopropyl | form) | | | |
| | oleic acid amide | | | | |
| | (example 9) | | | | |
| 10.j | N,N- | 7 (—COOH | 5 | 84.5 | 79.9 |
| | dimethylaminopropyl | form) | | | |
| | oleic acid amide | | | | |
| | (example 9) | | | | |
| 10.k | Polyquaternium-16 | 3 (—COO⁻ | 5 | 72.2 | 87.9 |
| | | Na⁺ form) | | | |
| 10.l | tetrabutyl ammonium | 2 (—COOH | 5 | 27.0 | 57.6 |
| | bromide | form) | | | |
| 10.m | tetrabutyl ammonium | 2 (—COO⁻ | 5 | 69.4 | 79.4 |
| | bromide | Na⁺ form) | | | |

The combing force data in the above table on damaged human hair show that the salt compounds according to the invention are able to reduce significantly the combing forces on different keratinous substrates.

The data demonstrate that this combing force reduction can be achieved for a broad variety on poly fatty acid based carboxylate structures.

Linear structures derived from unsaturated fatty acids (synthesis examples 2 and 3) as well as mixed unsaturated/saturated fatty acid systems (synthesis examples 2a, 2b, 2c) can be used. Further, the branched/dendritic derivative (examples 4a) performs well.

The same is true for castor oil derived branched poly fatty acid carboxylates (example 7)

Non monofunctional poly fatty acid based carboxylates, i.e. bis-carboxylic acid based poly fatty acid structures (example 6) are effective on human hair too.

The data also demonstrate that this combing force reduction can be achieved for a broad variety on amino/ammonium structures.

Alkyl modified primary amines (oleylamine) as well as betaine like primary amines (lysine) can be used.

Further, alkyl modified tertiary amines (example 9) can be utilized.

Mono-quaternary compounds, such as tetrabutyl ammonium compounds, and poly-quaternized polymers, i.e. polyquaternium 16, in combination with poly fatty acid based anions can also yield significant combing force reductions.

With respect to mono-quaternary compounds the way of formation of the poly fatty acid carboxylate salts has a significant influence.

A comparison of the tetrabutyl ammonium bromide-based examples 10.1 (poly fatty acid in COOH form; no true salt formation with the tetrabutyl ammonium bromide R₄N⁺ Br⁻) and 10.m (poly fatty acid in —COO⁻ Na⁺ form; true salt formation with the tetrabutyl ammonium bromide R₄N⁺ Br⁻) highlights the difference. The use of the poly fatty acid in —COO⁻ Na⁺ form according to the invention yields a strong reduction of the dry tear off force as well as wet average force. In contrast, the non inventive usage of the poly fatty acid in —COOH form yields a significantly lower reduction of the dry tear off force as well as the wet average force. Further, an expert panel evaluation of the hand feel of the hair tresses surfaced significant differences. The non inventive hair treatment with the poly fatty acid in COOH form yields a negatively judged dull hand feel compared to a smooth hand feel after treatment with the poly fatty acid in —COO⁻ Na⁺ form.

True salt formation with polyquaternium 16 in 10.k also results in a reduction of the dry tear off force as well as the wet average force. Further, an expert panel evaluation of the hand feel of the hair tresses surfaced significant differences. Similar to what was found for the tetrabutyl ammonium bromide-based experiments 10.l and 10.n, the inventive hair treatment with the poly fatty acid in —COO⁻ Na⁺ form yields a smooth hand feel after treatment with the poly fatty acid (10.k).

Based on the above outlined considerable flexibility with respect to the poly fatty acid based carboxylate structure as well as the amino/ammonium structure suitable salt compounds for a broad variety of specific applications can be developed.

1.-59. (canceled)

60. An organic ammonium salt comprising
an organic ammonium-group-comprising cation,
with the proviso that said organic ammonium-group-comprising cation does not comprise moieties of the formulas (III) or (IV):
(—Z—C(O)—R6)r—Z—C(O)— (III) or
(—C(O)—Z—R6)r—C(O)—Z— (IV),
wherein
Z can be the same or different and is selected from —O—, or —NR11-, wherein
R11 is independently selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups 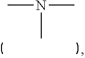
and can be substituted with one or more hydroxyl and halide groups,
R6 is independently selected from optionally substituted straight-chain, cyclic or branched, saturated or unsaturated hydrocarbon radicals which have 1 to 36 carbon atoms, with the proviso that at least one R6 has more than 6 carbon atoms, and
r is 1 to 20,
and a carboxylate anion (COO—) group-comprising anion selected from the group consisting of anions of the formulae (V), (VII), and (X):
formula (V):
R7(—X—C(O)-G)p (V)
wherein
R7 in formula (V) is selected from a p-valent, optionally substituted hydrocarbon radical and may contain optionally one or more groups selected from —O—, —NH—, —C(O)—, —C(S)—, tertiary amino groups 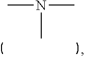
and quaternary ammonium groups, and can be optionally substituted by one or more substituent groups selected from a carboxyl group (—COOH) group, a carboxylate anion (—COO—) group and a hydroxyl (—OH) group,
p≥1,
X can be the same or different and is selected from —O—, or —NR10-, wherein R10 is selected from the group consisting of hydrogen, or optionally substituted straight-chain, cyclic or branched, saturated, unsaturated or aromatic hydrocarbon radicals which have up to 100 carbon atoms which optionally contain one or more groups selected from —O—, —NH—, —C(O)—, —C(S)—, tertiary amino groups 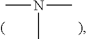
or in formula (V) R10 may form a bond to R7 to form a cyclic structure,
G can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups 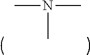
and can be optionally substituted by one or more substituent groups selected from a carboxyl (—COOH) group, a carboxylate anion (—COO—) group, a hydroxyl (—OH) group and a halide (-halogen) group,
with the proviso that at least one of the radicals G contains at least one moiety of the formula (VI) or (VI*):
—R8(—X—C(O)—R8)m—X—C(O)—R9 (VI)
—R8(—X—C(O)—R8)m—X—C(O)—R9* (VI*)
wherein
X is as defined above,
m=0 to 20,
R8 is independently selected from a divalent optionally substituted straight-chain, cyclic or branched, saturated or unsaturated hydrocarbon radical which have up to 36 carbon atoms,
R9 is independently selected from optionally substituted straight-chain, cyclic or branched, saturated or unsaturated hydrocarbon radicals which have 1 to 1000 carbon atoms, optionally containing one or more groups selected from —O—, —NH—, —C(O)—, —C(S)—, tertiary amino groups 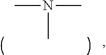
quaternary ammonium groups 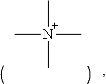
and which can be substituted with one or more substituent groups selected from a carboxyl (—COOH) group, carboxylate anion (—COO—) group, a hydroxyl (—OH) group, and a halide (-halogen) group, wherein the radical R9 cannot contain an internal carboxy (—COO—) group or (—CON(R′)—, R′ being hydrogen or organic group) amide group, i.e. R9 cannot contain a combination of a —C(O)— group and a —O— group or a combination of a —C(O)— group and a —NH— or tertiary amino group, and
with the proviso that in at least one moiety of the formula (VI) R9 has at least 2 carbon atoms, and that in the same moiety of the formula (VI) at least one R8 has at least 6 carbon atoms,
with the proviso, that at least one of R7 and G comprises one or more carboxylate anion (—COO—) groups,
R9* is independently selected from optionally substituted branched or dendrimeric hydrocarbon radicals which have 1 to 1000 carbon atoms, optionally containing one or more groups selected from —O—, —NH—, —C(O)—, —C(S)—, tertiary amino groups 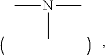
quaternary ammonium groups 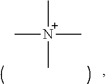
and which can be substituted with carboxyl, hydroxyl, or halide groups, wherein the radical R9* is terminated by two or more groups of the general structure
—X—C(O)-T
wherein X is as defined above, and
T is a monovalent straight-chain, cyclic or branched, saturated or unsaturated hydrocarbon radical optionally substituted with carboxyl, hydroxyl, or halide groups with up to 36 carbon atoms,
with the proviso that in at least one moiety of the formula (VI*) R9* is terminated by one or more groups T having at least 2 carbon atoms, and in the same moiety of the formula (VI*) at least one R8 has at least 6 carbon atoms,
and with the proviso, that at least one of R7 and G in formula (V) comprises one or more carboxylate anion (—COO—) groups,
formula (VII):
R7(—C(O)—X—Y)q (VII),
wherein
R7 and X are as defined above,
q=1 to 55, and
Y can be the same or different and is selected from optionally substituted straight-chain, cyclic or branched, saturated, unsaturated or aromatic hydrocarbon radicals which have up to 1005 carbon atoms, which optionally contain one or more groups selected from —O—, —NH—, —C(O)—, —C(S)—, and tertiary amino groups 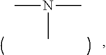
and can be substituted by one or more substituent groups selected from a carboxyl (—COOH) group, a carboxylate anion (—COO—) group and a hydroxyl group,
with the proviso that at least one of the radicals Y contains at least one moiety of the formula (VIII) or (VIII*):
—R8(—C(O)—X—R8)m—C(O)—X—R9 (VIII)
—R8(—C(O)—X—R8)m—C(O)—X—R9* (VIII*)
wherein X, m, R8, and R9 in formula (VIII) are each as defined above for formula (VI), and
X, m, R8, and R9* in formula (VIII*) are each as defined above for formula (VI*),
and with the proviso that at least one of R7 and Y in formula (VII) comprises one or more carboxylate anion (—COO—) groups,
formula (X):
R7(—C(O)—X—R8-COO—)q (X)
wherein X, R7, R8, and q in formula (X) are each as defined above for formula (VII) and (VIII).

61. An organic ammonium salt according to claim 60, wherein said organic ammonium-group-comprising cation is selected from cations of the formula (I):
R1(—F)x (I),
wherein
x is 1 to 50,
R1 is selected from x-valent, optionally substituted hydrocarbon radicals which have up to 1000 carbon atoms, and may contain optionally one or more groups selected from —O—, —NH—, —C(O)—, —C(S)—, tertiary amino groups 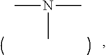
and R1 can be substituted by one or more groups selected from a carboxyl (—COOH) group, a carboxylate anion (—COO—) group, a hydroxyl (—OH) group and a halide (-halogen) group, and
F can be the same or different and is represented by the general formula (II) 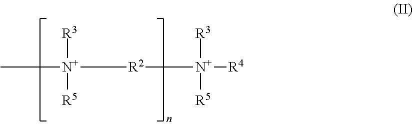
wherein the groups F bind to a carbon atom of R1, and
n is independently 0 to 1000,
R2 can be the same or different and is selected from divalent optionally substituted hydrocarbon radicals which have up to 1000 carbon atoms, and optionally contain one or more groups selected from —O—, —NH—, —C(O)—, —C(S)—, tertiary amino groups 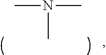
and R2 can be substituted with one or more groups selected from OH groups and halide groups, and
R3, R4, R5 can be the same or different and are selected from hydrogen and optionally substituted straight-chain, cyclic or branched, saturated, unsaturated or aromatic hydrocarbon radicals which have up to 1000 carbon atoms, which optionally contain one or more groups selected from —O—, —NH—, —C(O)—, —C(S)—, tertiary amino groups 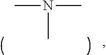
quaternary ammonium groups 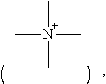
and which can be substituted with one or more groups selected from OH groups and halide groups,
wherein if R3, R4, R5 are not hydrogen they each bind with a carbon atom to the nitrogen atom.

62. An organic ammonium salt according to claim 60, wherein said organic ammonium-group-comprising cation is selected from the group of
a. mono and polyquaternium cations,
b. basic amino acid cations,
c. mono and poly tertiary amine based cations
d. mono and poly secondary amine based cations
e. mono and poly primary amine based cations.

63. An organic ammonium salt according to claim 60, wherein said organic ammonium-group-comprising cation has at least 6 carbon atoms.

64. An organic ammonium salt according to claim 60, wherein the carboxylate anion (COO—) group-comprising anion or anions are selected from a group consisting of polymeric fatty acid carboxylates of the type
R7[(—C(O)—X—R8)m+1-C(O)—X—R9]q or
R7[(X—C(O)—R8)m+1-X—C(O)—R9]p,
wherein X, R7, R8, R9, m, p and q are as defined above for the formulas (V), (VI), (VII) and (VIII) and wherein either R7 or at least one of R9, or both R7 and at least one of R9 bear one or more carboxylate groups.

65. An organic ammonium salt according to claim 60, wherein the carboxylate anion (COO—) group-comprising anion or anions of the organic ammonium salt contain at least one moiety of the general formula (VIa)
(—X—C(O)—R8)m—X—C(O)—R9 (VIa),
or of the general formula (VIIIa)
(—C(O)—X—R8)m—C(O)—X—R9 (VIIIa)
wherein X and R8 and m are as defined above, and
R9 is independently selected from optionally substituted straight-chain, cyclic or branched, saturated or unsaturated hydrocarbon radicals which have 1 to 36 carbon atoms, optionally containing one or more groups selected from —O—, —NH—, —C(O)—, —C(S)—, and which can be substituted with OH groups, carboxylate groups or halide groups.

66. An organic ammonium salt according to claim 60, wherein at least one group R9 radical is selected from linear alkyl groups and linear alkenyl groups.

67. An organic ammonium salt according to claim 60, wherein at least one group R9 is derived from a carboxylic acid or a hydroxycarboxylic acid bearing one or more hydroxylic groups.

68. An organic ammonium salt according to claim 60, wherein each moiety of the general formula (VI), (VI*), (VIII) or (VIII*) contains at least one R8 selected from optionally hydroxyl-substituted hexylene, heptylene, octylene, nonylene, decylene, undecylene, dodecylene, tridecylene, tetradecylene, pentadecylene, hexadecylene, heptadecylene, octadecylene, nonadecylene, eicosylene, henicosylene, doicosylene, tricosylene, and tetraicosylene, or hexenylene, heptenylene, octenylene, nonenylene, decenylene, undecenylene, dodecenylene, tridecenylene, tetradecenylene, pentadecenylene, hexadecenylene, heptadecenylene, octadecenylene, nonadecenylene, eicosenylene, henicosenylene, doicosenylene, tricosenylene, and tetraicosenylene.

69. An organic ammonium salt according to claim 60, wherein the carboxylate anion (COO—) group-comprising anion or anions comprise branched linear polymeric fatty acid carboxylates, containing at least one moiety of the formula
Glycerol-O—C(O)—R8-(O—C(O)—R8)m—O—C(O)—R9
wherein
R8 is as defined above,
R9 is as defined above,
and in the moieties of the above-shown formula
m=0 to 20, and
the total number of carbon atoms in R8+R9 (□□carbon atoms of R8 and R9) being 19 to 300.

70. An organic ammonium salt according to claim 60, wherein the carboxylate anion (COO—) group-comprising anion or anions comprise branched or dendritic polymeric fatty acid carboxylate anions A− of the formula (V) wherein in the formula (VI) or (VI*)
R7, X, R8, R9, R9* and m are as described above, and
wherein the R7 group bears at least one anionic carboxylate group,
R8 is as defined above,
R9 is as defined above,
and
m=1 to 20, and
the total number of carbon atoms in R8+R9 (□□carbon atoms of R8 and R9) being 19 to 300.

71. An organic ammonium salt according to claim 60, wherein the carboxylate anion (COO—) group-comprising anion or anions—comprise carboxylate anions A− of the type
R7[(—C(O)—X—R8)m+1-C(O)—X—R9C(O)O—]q, or (—OC(O))q−1-R7-(C(O)—X—R8)m+1-C(O)—X—R9C(O)O—
and wherein X=O, R7, R8, R9, m and q are as defined above, and
R8 is as defined above,
R7 is as defined above,
and
m=0 to 20, and
the total number of carbon atoms in R8+R9 (□□carbon atoms of R8 and R9 per anion) is 19 to 300.

72. An organic ammonium salt according to claim 61, wherein the organic ammonium-group-comprising cation is selected from cations according to the general formula
R1(-F)x (I) as defined above,
wherein
x is 1 to 10,
F can be the same or different and is represented by the general formula (II): 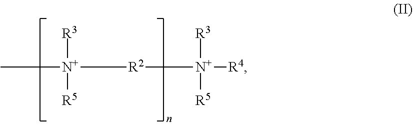
wherein R1, R2, R3, R4, R5 and n are as defined above.

73. An organic ammonium salt according to claim 61, wherein the organic ammonium-group-comprising cation is selected from cations according to the general formula
R1(-F)x (I)
as defined above, wherein
x=2 and the cation is represented by the general formula (V): 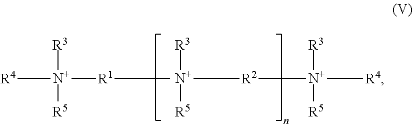
wherein R1, R2, R3, R4, R5 and n are as defined above.

74. An organic ammonium salt according to claim 60, wherein
the carboxylate anion (COO—) group-comprising anion or anions comprise at least one moiety of the general formulas (XI) or (XII):
—X—C(O)-Rx-(X—C(O)-Rx)m—X—C(O)—R9 (XI) or
—X—C(O)-Rx-(X—C(O)-Rx)m—X—C(O)—R9 (XII)
wherein
X is O or NR11,
m=1 to 20, and
the total number of carbon atoms in Rx+R9 (□carbon atoms Rx, R9 per anion) is 19 to 300,
R11 is as defined above,
Rx is an optionally OH, —O—C(O)—R9, —O—C(O)—R8-(O—C(O)—R8)0-19-O—C(O)—R9 substituted straight-chain, cyclic or branched, saturated or unsaturated hydrocarbon radical which has 1 to 36 carbon atoms excluding the carbon atoms of R8 and R9 group-containing substituents,
R8 is as defined above,
R9 is selected from optionally substituted straight-chain, cyclic or branched, saturated or unsaturated hydrocarbon radicals which have 1 to 36 carbon atoms.

75. An organic ammonium salt according to claim 60, wherein in at least one of the moieties of the formula (VI), (VI*) or (VIII), (VIII*) of the carboxylate anion (COO—) group-comprising anion of the formula (V) or (VII) two or more different R8 groups are present.

76. An organic ammonium salt according to claim 60, wherein in at least one of the moieties of the formula (VI), (VI*) or (VIII), (VIII*) of the carboxylate anion (COO—) group-comprising anion of the formula (V) or (VII) the groups R8 and R9 or R8 and R9* are not based on the same carboxylic acid structure.

77. An organic ammonium salt according to claim 60, wherein in formula (V) p is 2-6,
R7 is selected from di- to hexavalent linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups,
and at least one group G contains one or more moieties of the general formula (VI*)
—R8(—X—C(O)—R8)m—X—C(O)—R9* (VI*)
wherein R8, R9* and m are as defined above,
or wherein
in formula (VII) q is 2-6,
R7 is selected from di- to hexavalent linear, branched or cyclic alkylene groups, linear, branched or cyclic alkenylene groups, linear, branched or cyclic alkynylene groups, linear, branched or cyclic alkarylene groups, linear, branched or cyclic aralkylene groups and linear, branched or cyclic arylene groups,
and at least one group Y contains one or more moieties of the general formula (VIII*)
—R8(—C(O)—X—R8)m—C(O)—X—R9* (VIII*)
wherein R8, R9* and m are as defined above.

78. An organic ammonium salt according to claim 77, wherein in the carboxylate anion (COO—) group-comprising anion of the formula (V) or (VII) one or more groups R9* are each terminated by three or more groups —O—C(O)-T, wherein T is as defined above.

79. An organic ammonium salt according to claim 77, wherein in the carboxylate anion (COO—) group-comprising anion of the formula (V) or of the formula (VII) one or more groups R9* each contain at least two branching structures of the general formula
—C(O)—B(—O—)_{b},
wherein B is a linear or branched hydrocarbon group having 2-20 carbon atoms, and b is 2 or more, and wherein the b groups (—O—) linked to the group B on the one side are linked to a C atom which may be the C atom of a CH₂ group or of a carbonyl group on the other side.

80. An organic ammonium salt according to claim 60, wherein in the carboxylate anion (COO—) group-comprising anion of the formula (V) or (VII) one or more groups R9* are each terminated by two or more groups of the structure
—R8(—X—C(O)—R8)t—X—C(O)-T,
wherein R8, and T are as defined above, and
X=O,
t is independently 0-12.

81. An organic ammonium salt according to claim 60, wherein
in the carboxylate anion (COO—) group-comprising anion of the formula (V) containing one or more moieties of the general formula (VI*)
—R8(—X—C(O)—R8)m—X—C(O)—R9* (VI*)
wherein R8, R9* and m are as defined above,
or in the carboxylate anion (COO—) group-comprising anion of the formula (VII) containing one or more moieties of the general formula (VIII*)
—R8(—C(O)—X—R8)m—C(O)—X—R9* (VIII*)
wherein R8, R9* and m are as defined above,
one or more groups R9* are independently selected from one of the following branched or dendrimeric fatty acid structures:
-R14-O—C(O)—R15-(O—C(O)—R16)m1-(O—C(O)—R17)m2-O—C(O)-T or
-R14-NR10-C(O)—R15-(O—C(O)—R16)m1-(O—C(O)—R17)m2-O—C(O)-T,
wherein
R10 is as defined above,
R14 is selected from divalent optionally substituted hydrocarbon radicals which 2 to and 50 carbon atoms and may contain optionally one or more groups selected from —O—, —NH—, —C(O)—, —C(S)—, tertiary amino groups 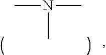
and can be substituted by —OH or halide groups, wherein the radical R12 cannot contain a combination of a —C(O)— group and a —O— group or a combination of a —C(O)— group and a —NH— or tertiary amino group forming an internal carboxylate group or an internal amide group,
R15 is independently selected from a divalent optionally substituted straight-chain, cyclic or branched, saturated or unsaturated hydrocarbon radical which have up to 36 carbon atoms or from a C2 to C24 monocarboxylic acid having 2 to 6 hydroxy groups,
m1 is 0 to 12,
m2 is Oto 12,
and m1+m2 is t, wherein t is 0 to 12, and
T is as defined above,
R81 and R82 are selected from the groups R8 as defined above,
wherein the total number of carbon atoms in R15+T (□carbon atoms R15, T) is 19 to 300,
with the proviso that for R15, R16 and R17 being derived from di- or polyhydroxylated carboxylic acids at least one OH groups are esterified.

82. An organic ammonium salt according to claim 60, wherein in the carboxylate anion (COO—) group-comprising anion of the formula (V) is represented by the following schematic ester structure:
—O(O)C—(C1-C12 radical)-C(O)—O-(mono or oligo C8-C24 hydroxy fatty acid)-C(O)—O—(C2-C10 hydrocarbon)-O—C(O)-(mono or oligo C8-C24 hydroxy fatty acid)-O—C(O)—(C1-C12 radical)-C(O)O—,
wherein
the terminal C1-C12 radicals are selected from linear, branched, saturated, unsaturated or aromatic C1-C12 radicals.

83. An organic ammonium salt according to claim 60, wherein the carboxylate anion (COO—) group-comprising anion of the formula (V) or (VII) comprises
at least one moiety of the general formula
—([—O—C(O)—R8(—O—C(O)—R8)l—O—C(O)-L-C(O)—O—(R8-C(O)—O)l-R8-C(O)O])—
wherein R8 is as defined above,
l is an integer independently selected from 0-20, and
L is a divalent hydrocarbon radical which may have 1 to 30 carbon atoms and may contain optionally one or more groups selected from —O—, —S—, —NH—, —C(O)—, —C(S)—, tertiary amino groups 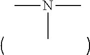
and quaternary ammonium groups.

84. An organic ammonium salt according to claim 60, wherein at least one of the groups R7, G or Y of the carboxylate anion (COO—) group-comprising anion of the formula (V) or (VII) contains at least one moiety of the formula
R1*[(—O—C(O)—R8)m—O—C(O)-]2,
wherein R1* is a divalent C1-C100 radical,
m is independently selected from 1 to 12, and
R8 is as defined above.

85. A process for the manufacture of the organic ammonium salt according to claim 60, which comprises:
(i) the reaction of an organic amine corresponding to the organic ammonium-group-comprising cation with a carboxylic acid (—COOH) corresponding to the carboxylate anion (COO—) group-comprising anion selected from the group consisting of the formulas (V), (VII), and (X); or
(ii) the reaction of an inorganic anion-containing salt of the organic ammonium-group-comprising cation with a metal salt of the carboxylic acid (—COOH) corresponding to the carboxylate anion (COO—) group-comprising anion selected from the group consisting of the formulas (V), (VII), and (X); or
(iii) the reaction of a hydroxy salt of the organic ammonium-group-comprising cation with the carboxylic acid corresponding to the carboxylate anion (COO—) group-comprising anion selected from the group consisting of the formulas (V), (VII) and (X).

86. The process according to the previous claim 85, wherein the manufacture of the organic ammonium salt comprises the anion exchange reaction step (ii), and wherein alkali metal salts or alkaline earth metal salts of the carboxylic acid (—COOH) corresponding to the carboxylate anion (COO—) group-comprising anion selected from the group consisting of the formulas (V), (VII), and (X) are applied.

87. The process according to claim 85, wherein the manufacture of the organic ammonium salt comprises the anion exchange reaction step (iii), and wherein the anion exchange reaction step (iii) of contacting the carboxylic acid corresponding to the carboxylate anion (COO—) group-comprising anion selected from the group consisting of the formulas (V), (VII), and (X) with a primary, secondary, tertiary and quaternary ammonium ion having an OH— counter ion is carried out in a separate reaction step prior to any contact with other ingredients of the final cosmetic formulations.

88. A cosmetic formulation for skin care and for hair care, a polishing agent for treating and coating hard surfaces, in a formulation for drying automobiles and other hard surfaces, for finishing textiles and textile fibers, as separate softeners for use after textiles have been washed with nonionic or anionic/nonionic detergent formulations, as softeners in formulations for washing textiles that are based upon nonionic or anionic/nonionic surfactants, and as means for preventing or removing wrinkles in textiles, which comprise the compound of claim 60.

89. A composition for the treatment of hair selected from the group consisting of hair shampoo compositions, hair conditioning compositions, hair coloration or dyeing compositions, hair combability improving compositions, hair rinse-off and leave-on compositions comprising the compound of claim 60.
